(19) United States
(12) Patent Application Publication
(10) Pub. No.: US 2011/0097717 A1
(43) Pub. Date: Apr. 28, 2011

(54) Gene Expression Profiling For Identification of Cancer

(76) Inventor: Danute BANKAITIS-DAVIS, Longmont, CO (US)

(21) Appl. No.: 12/741,787

(22) PCT Filed: Nov. 6, 2007
(86) PCT No.: PCT/US07/23459

§ 371 Date: Dec. 9, 2010

(51) Int. Cl. C12Q 1/68

(52) U.S. Cl. 435/6

(57) ABSTRACT

A method is provided for determining whether an individual has a particular cancer based on a sample from the subject, wherein the sample provides a source of RNAs. The method includes using amplification for measuring the amount of RNA corresponding to at least 1 constituent from Tables A-C.

### FIELD OF THE INVENTION

The present invention relates generally to the identification of biological markers associated with the identification of cancer. More specifically, the present invention relates to the use of gene expression data to distinguish between the presence of different cancers

### BACKGROUND OF THE INVENTION

The term cancer collectively refers to more than 100 different diseases that affect nearly every part of the body. Throughout life, healthy cells in the body divide, grow, and replace themselves in a controlled fashion. Cancer starts when the genes directing this cellular division malfunction, and cells begin to multiply and grow out of control. A mass or clump of these abnormal cells is called a tumor. Not all tumors are cancerous. Benign tumors, such as moles, stop growing and do not spread to other parts of the body. But cancerous, or malignant, tumors continue to grow, crowding out healthy cells, interfering with body functions, and drawing nutrients away from body tissues. Malignant tumors can spread to other parts of the body through a process called metastasis. Cells from the original tumor break off, travel through the blood or lymphatic vessels or within the chest, abdomen or pelvis, depending on the tumor, and eventually form new tumors elsewhere in the body.

Only 5-10% of cancers are thought to be hereditary. The rest of the time, the genetic mutation that leads to the disease is brought on by other factors. The most common cancers are linked to smoking, sun exposure, and diet. These factors, combined with age, family history, and overall health, contribute to an individual's cancer risk.

Several diagnostic tests are used to rule out or confirm cancer. For many cancers, a biopsy is the primary diagnostic tool. However, many biopsies are invasive, unpleasant procedures with their own associated risks, such as pain, bleeding, infection, and tissue or organ damage. In addition, if a biopsy does not result in an accurate or large enough sample, a false negative or misdiagnosis can result, often requiring that the biopsy be repeated. What is needed are improved methods to specifically detect and characterize specific types of cancer. These methods must also be able to distinguish between different types of cancers.

### SUMMARY OF THE INVENTION

The present invention provides molecular markers capable of discriminating between cancer types. Specifically, the invention is based upon the discovery of identification of gene expression profiles (Precision Profiles™) associated with cancer. Cancer includes for example, breast cancer, ovarian cancer, cervical cancer, prostate cancer, lung cancer, colon cancer or skin cancer. These genes are referred to herein as cancer associated genes or cancer associated constituents. More specifically, the invention is based upon the surprising discovery that detection of as few as one cancer-associated gene in a subject derived sample is capable of distinguishing between cancer types with at least 75% accuracy. More particularly, the invention is based upon the surprising discovery that the methods provided by the invention are capable of detecting cancer by assaying blood samples.

In various aspects the invention provides methods of evaluating the presence of a particular cancer type based on a sample from the subject, the sample providing a source of RNAs, and determining a quantitative measure of the amount of at least one constituent of any constituent (e.g., cancer-associated gene) of any of Tables A, B, and C and arriving at a measure of each constituent.

The methods of the invention further include comparing the quantitative measure of the constituent in the subject derived sample to a reference value or a baseline value, e.g. baseline data set. The reference value is for example an index value. Comparison of the subject measurements to a reference value allows for the present of a particular cancer type to be determined.

The baseline data set or reference values may be derived from one or more other samples from the same subject taken under circumstances different from those of the first sample, and the circumstances may be selected from the group consisting of (i) the time at which the first sample is taken (e.g., before, after, or during treatment cancer treatment), (ii) the site from which the first sample is taken, (iii) the biological condition of the subject when the first sample is taken.

The measure of the constituent is increased or decreased in the subject compared to the expression of the constituent in the reference, e.g., normal reference sample or baseline value. The measure is increased or decreased 10%, 25%, 50% compared to the reference level. Alternately, the measure is increased or decreased 1, 2, 5 or more fold compared to the reference level.

In various aspects of the invention the methods are carried out wherein the measurement conditions are substantially repeatable, particularly within a degree of repeatability of better than ten percent, five percent or more particularly within a degree of repeatability of better than three percent, and/or wherein efficiencies of amplification for all constituents are substantially similar, more particularly wherein the efficiency of amplification is within ten percent, more particularly wherein the efficiency of amplification for all constituents is within five percent, and still more particularly wherein the efficiency of amplification for all constituents is within three percent or less.

In addition, the one or more different subjects may have in common with the subject at least one of age group, gender, ethnicity, geographic location, nutritional history, medical condition, clinical indicator, medication, physical activity, body mass, and environmental exposure. A clinical indicator may be used to assess cancer or a condition related to cancer of the one or more different subjects, and may also include interpreting the calibrated profile data set in the context of at least one other clinical indicator, wherein the at least one other clinical indicator includes blood chemistry, X-ray or other radiological or metabolic imaging technique, molecular markers in the blood, other chemical assays, and physical findings.

At least 1, 2, 3, 4, 5, 6, 7, 8, 9, 10, 15, 20, 30 40, 50 or more constituents are measured. Preferably, at least one constituent is measured.

For example, where the constituent is selected from the Precision Profile™ for Inflammatory Response (Table A), LTA, IFI16, PTPRC, CD86, ADAM17, HMOX1, TXNRD1, MYC, MHC2TA, MAPK14, TLR2, CD19, TNFRSF1A, TIMP1, TNF, IL23A, HLADRA, TLR4, PLAUR, PTGS2, PLA2G7, CCR5, or TOSO is measured such as to distinguish between a breast cancer diagnosed subject and a colon cancer diagnosed subject in a reference population; IFI16, TIMP1, MAPK14, LTA, TGFB1, HMOX1, TNFRSF1A, PTPRC, PLAUR, EGR1, ADAM17, TLR2, MYC, SSI3, TNF, CD86, IL1B, CCL5, MHC2TA, CXCR3, TXNRD1, PTGS2, ICAM1, IL1RN, SERPINE1, CD4, NFKB1, CCR5, TLR4, IL18BP, CCL3, HLADRA, MMP9, or IL32 is measured such as to distinguish between a breast cancer diagnosed subject and a melanoma cancer diagnosed subject in a reference population; TIMP1, MAPK14, SSI3, PTPRC, or IL1RN is measured such as to distinguish between a breast cancer diagnosed subject and an ovarian cancer diagnosed subject in a reference population; IRF1, ICAM1, TIMP1, PTGS2, TGFB1, TNFRSF1A, CXCL1, or IFI16 is measured such as to distinguish between a breast cancer diagnosed subject and a cervical cancer diagnosed subject in a reference population; or ELA2, VEGF, TIMP1, PTPRC, MMP9, IL1R1, PTGS2, TXNRD1, IL10, HSPA1A, IL1RN, and ALOX5, APAF1, CXCL1, TNF, MAPK14, or EGR1 is measured such as to distinguish between a breast cancer diagnosed subject and a lung cancer diagnosed subject in a reference population. Wherein the constituent is selected from the Human Cancer General Precision Profile™ (Table B), EGR1, TGFB1, NFKB1, SRC, TP53, ABL1, SERPINE1, or CDKN1A is measured such as to distinguish between a breast cancer diagnosed subject and a melanoma cancer diagnosed subject in a reference population; TIMP1, MMP9, CDKN1A, or IFITM1 is measured such as to distinguish between a breast cancer diagnosed subject and an ovarian cancer diagnosed subject in a reference population; NME4, TIMP1, BRAF, ICAM1, PLAU, RHOA, IFITM1, TNFRSF1A, NOTCH2, TGFB1, SEMA4D, MMP9, FOS, TNF, MYC, AKT1, or EGR1 is measured such as to distinguish between a breast cancer diagnosed subject and a cervical cancer diagnosed subject in a reference population; or BRAF, PLAU, RHOA, RB1, TIMP1, CDKN1A, SMAD4, S100A4, NME4, MMP9, IFITM1, PTEN, VEGF, NRAS, TNF, TGFB1, BRCA1, SEMA4D, CDK5, TNFRSF1A, or EGR1 is measured such as to distinguish between a breast cancer diagnosed subject and a lung cancer diagnosed subject in a reference population. Wherein the constituent is selected from the Precision Profile™ for EGR1 (Table C), TGFB1, EGR1, SMAD3, NFKB1, SRC, TP53, NFATC2, PDGFA, or SERPINE1 is measured such as to distinguish between a breast cancer diagnosed subject and a melanoma cancer diagnosed subject in a reference population; ALOX5 or EP300 is measured such as to distinguish between a breast cancer diagnosed subject and an ovarian cancer diagnosed subject in a reference population; ALOX5, CREBBP, EP300, MAPK1, ICAM1, PLAU, TGFB1, CEBPB, FOS, or SMAD3 is measured such as to distinguish between a breast cancer diagnosed subject and a cervical cancer diagnosed subject in a reference population; or EP300, PLAU, MAPK1, ALOX5, CREBBP, TOPBP1, PTEN, S100A6, TGFB1, or EGR1 is measured such as to distinguish between a breast cancer diagnosed subject and a lung cancer diagnosed subject in a reference population.

In another aspect, wherein the constituent is selected from the Precision Profile™ for Inflammatory Response (Table A), IFI16, LTA, TNFRSF1A, PTPRC, VEGF, TNF, TIMP1, CD86, PLAUR, PTGS2, ADAM17, MYC, TGFB1, IL1RN, HMOX1, TLR4, TLR2, MNDA, MAPK14, TXNRD1, ICAM1, CASP3, IL1B, CCL5, NFKB1, HLADRA, SSI3, SERPINA1, HSPA1A, MMP9, SERPINE1, MHC2TA, CXCR3, PLA2G7, CCR5, CD19, or EGR1 is measured such as to distinguish between a cervical cancer diagnosed subject and a colon cancer diagnosed subject in a reference population; IFI16, PLAUR, TGFB1, TNFRSF1A, LTA, TIMP1, MAPK14, ICAM1, IL1RN, PTPRC, IL1B, ADAM17, PTGS2, CCL5, TNF, EGR1, SSI3, HMOX1, MYC, CD86, IRF1, MNDA, TLR2, NFKB1, SERPINE1, HSPA1A, SERPINA1, TXNRD1, MMP9, VEGF, TLR4, CASP3, CXCR3, CD4, CCL3, CASP1, MHC2TA, CCR5, TNFSF5, HLADRA, IL18BP, IL1R1, or IL32, is measured such as to distinguish between a cervical cancer diagnosed subject and a melanoma cancer diagnosed subject in a reference population; LTA is measured such as to distinguish between a cervical cancer diagnosed subject and an ovarian cancer diagnosed subject in a reference population; RF1, ICAM1, TIMP1, PTGS2, TGFB1, TNFRSF1A, CXCL1, or IFI16 is measured such as to distinguish between a cervical cancer diagnosed subject and a breast cancer diagnosed subject in a reference population; or CASP3, IL18, TXNRD1, or IFNG is measured such as to distinguish between a cervical cancer diagnosed subject and a lung cancer diagnosed subject in a reference population. Wherein the constituent is selected from the Human Cancer General Precision Profile™ (Table B), NME4, BRAF, NFKB1, SMAD4, ABL2, RHOA, NOTCH2, TIMP1, TGFB1, SEMA4D, BCL2, CDK2, NRAS, RB1, CDK5, IL1B, or FOS is measured such as to distinguish between a cervical cancer diagnosed subject and a colon cancer diagnosed subject in a reference population; EGR1, ICAM1, TGFB1, SERPINE1, NME4, NFKB1, SEMA4D, TIMP1, TNF, BRAF, NOTCH2, SRC, RHOA, IFITM1, FOS, CDKN1A, PLAUR, PLAU, TNFRSF1A, IL1B, E2F1, TP53, THBS1, MYC, ABL2, AKT1, MMP9, SOCS1, SMAD4, CDK5, CDK2, ABL1, RHOC, BRCA1, or BCL2 is measured such as to distinguish between a cervical cancer diagnosed subject and a melanoma cancer diagnosed subject in a reference population; MYCL1 or AKT1 is measured such as to distinguish between a cervical cancer diagnosed subject and an ovarian cancer diagnosed subject in a reference population; NME4, TIMP1, BRAF, ICAM1, PLAU, RHOA, IFITM1, TNFRSF1A, NOTCH2, TGFB1, SEMA4D, MMP9, FOS, TNF, MYC, AKT1, or EGR1 is measured such as to distinguish between a cervical cancer diagnosed subject and a breast cancer diagnosed subject in a reference population; or ITGB1 or RB1 is measured such as to distinguish between a cervical cancer diagnosed subject and a lung cancer diagnosed subject in a reference population. Wherein the constituent is selected from the Precision Profile™ for EGR1 (Table C), EP300, ALOX5, MAPK1, CREBBP, NFKB1, ICAM1, SMAD3, TGFB1, CEBPB, TOPBP1, NR4A2, FOS, or EGR1 is measured such as to distinguish between a cervical cancer diagnosed subject and a colon cancer diagnosed subject in a reference population; EGR1, ICAM1, PDGFA, TGFB1, EP300, SERPINE1, CREBBP, ALOX5, NFKB1, MAPK1, SRC, SMAD3, FOS, PLAU, CEBPB, TP53, THBS1, MAP2K1, NFATC2, NR4A2, EGR2, EGR3, TOPBP1, or CDKN2D is measured such as to distinguish between a cervical cancer diagnosed subject and a melanoma cancer diagnosed subject in a reference population; ALOX5, CREBBP, EP300, MAPK1, ICAM1, PLAU, TGFB1, CEBPB, FOS, or SMAD3 is measured such as to distinguish between a cervical cancer diagnosed subject and a breast cancer diagnosed subject in a reference population; or S100A6 is measured such as to distinguish between a cervical cancer diagnosed subject and a lung cancer diagnosed subject in a reference population.

In a further aspect, wherein the constituent is selected from the Precision Profile™ for Inflammatory Response (Table A), LTA, CD86, IFI16, PTPRC, VEGF, ADAM17, TXNRD1, TNF, MNDA, TIMP1, HMOX1, PTGS2, TNFRSF1A, IL1RN, TLR4, MYC, IL10, MAPK14, TLR2, PLAUR, TGFB1, ELA2, PLA2G7, IL1R1, NFKB1, IL1B, IL18, CXCR3, IL15, CCL5, HLADRA, EGR1, HSPA1A, IL5, ICAM1, SSI3, or IL8 is measured such as to distinguish between a lung cancer diagnosed subject and a colon cancer diagnosed subject in a reference population; IFI16, LTA, TIMP1, MAPK14, EGR1, ADAM17, PTPRC, HMOX1, CD86, TGFB1, CCL5, IL1RN, TNFRSF1A, TNF, PTGS2, IL1B, MNDA, PLAUR, TXNRD1, MYC, IL10, TLR2, SSI3, MMP9, VEGF, NFKB1, TLR4, ICAM1, SERPINE1, SERPINA1, HSPA1A, CXCR3, IL1R1, CCL3, IRF1, ELA2, CASP1, CCR5, CD4, IL18, MHC2TA, CXCL1, IL18BP, IL5, HLADRA, or TNFSF6 is measured such as to distinguish between a lung cancer diagnosed subject and a melanoma cancer diagnosed subject in a reference population; CASP3 or APAF1 is measured such as to distinguish between a lung cancer diagnosed subject and an ovarian cancer diagnosed subject in a reference population; CASP3, IL18, TXNRD1, or IFNG is measured such as to distinguish between a lung cancer diagnosed subject and a cervical cancer diagnosed subject in a reference population; ELA2, VEGF, TIMP1, PTPRC, MMP9, IL1R1, PTGS2, TXNRD1, IL10, HSPA1A, IL1RN, ALOX5, APAF1, CXCL1, TNF, MAPK14, or EGR1 is measured such as to distinguish between a lung cancer diagnosed subject and a breast cancer diagnosed subject in a reference population; or CCL5, EGR1, TGFB1, IL1RN, TIMP1, CCL3, TNF, PLAUR, IL1B, CXCR3, PTGS2, TNFRSF1A, PTPRC, NFKB1, ICAM1, CD8A, IRF1, IL32, HMOX1, SERPINA1, HSPA1A, or ALOX5 is measured such as to distinguish between a lung cancer diagnosed subject and a prostate cancer diagnosed subject in a reference population. Wherein the constituent is selected from the Human Cancer General Precision Profile™ (Table B), BRAF, NME4, RB1, SMAD4, NFKB1, RHOA, BRCA1, APAF1, NRAS, PLAU, CDK5, VEGF, TIMP1, BCL2, RAF1, TGFB1, SEMA4D, CFLAR, NOTCH2, or ABL2 is measured such as to distinguish between a lung cancer diagnosed subject and a colon cancer diagnosed subject in a reference population; EGR1, TGFB1, NFKB1, RHOA, BRAF, CDKN1A, TIMP1, TNF, PLAU, IFITM1, ICAM1, SEMA4D, THBS1, SERPINE1, NME4, NOTCH2, E2F1, SMAD4, MMP9, TP53, FOS, PLAUR, CDK5, IL1B, RB1, MYC, AKT1, SRC, TNFRSF1A, BRCA1, ABL2, PTCH1, CDK2, IGFBP3, CDC25A, SOCS1, WNT1, RHOC, PTEN, ITGB1, S100A4, ABL1, APAF1, VHL, or BCL2 is measured such as to distinguish between a lung cancer diagnosed subject and a melanoma cancer diagnosed subject in a reference population; TGB1 or RB1 is measured such as to distinguish between a lung cancer diagnosed subject and a cervical cancer diagnosed subject in a reference population; BRAF, PLAU, RHOA, RB1, TIMP1, CDKN1A, SMAD4, S100A4, NME4, MMP9, IFITM1, PTEN, VEGF, NRAS, TNF, TGFB1, BRCA1, SEMA4D, CDK5, TNFRSF1A, or EGR1 is measured such as to distinguish between a lung cancer diagnosed subject and a breast cancer diagnosed subject in a reference population; or EGR1, TGFB1, S100A4, RHOA, PLAUR, CDKN1A, TIMP1, WNT1, SEMA4D, E2F1, or SOCS1 is measured such as to distinguish between a lung cancer diagnosed subject and a prostate cancer diagnosed subject in a reference population. Wherein the constituent is selected from the Precision Profile™ for EGR1 (Table C), EP300, TOPBP1, ALOX5, NFKB1, MAPK1, CREBBP, PLAU, SMAD3, NAB1, MAP2K1, TGFB1, RAF1, or EGR1 is measured such as to distinguish between a lung cancer diagnosed subject and a colon cancer diagnosed subject in a reference population; EGR1, TGFB1, EP300, PDGFA, NFKB1, CREBBP, ALOX5, MAPK1, PLAU, SMAD3, ICAM1, THBS1, SERPINE1, MAP2K1, TP53, TOPBP1, FOS, NFATC2, SRC, CEBPB, CDKN2D, NR4A2, PTEN, EGR2, or EGR3 is measured such as to distinguish between a lung cancer diagnosed subject and a melanoma cancer diagnosed subject in a reference population; S100A6 is measured such as to distinguish between a lung cancer diagnosed subject and a cervical cancer diagnosed subject in a reference population; EP300, PLAU, MAPK1, ALOX5, CREBBP, TOPBP1, PTEN, S100A6, TGFB1, or EGR1 is measured such as to distinguish between a lung cancer diagnosed subject and a breast cancer diagnosed subject in a reference population; or EGR1, TGFB1, S100A6, EP300, or CREBBP is measured such as to distinguish between a lung cancer diagnosed subject and a prostate cancer diagnosed subject in a reference population.

In yet another aspect, wherein the constituent is selected from the Precision Profile™ for Inflammatory Response (Table A), LTA, IFI16, PTPRC, TNFRSF1A, TIMP1, MNDA, TLR2, IL1RN, VEGF, MAPK14, TLR4, TXNRD1, SSI3, PLAUR, PTGS2, TGFB1, HMOX1, IL1B, IL10, CASP3, ADAM17, or SERPINA1 is measured such as to distinguish between an ovarian cancer diagnosed subject and a colon cancer diagnosed subject in a reference population; IFI16, MAPK14, TNFRSF1A, TIMP1, PTPRC, TGFB1, IL1B, SSI3, IL1RN, LTA, PLAUR, MNDA, HMOX1, TLR2, PTGS2, ICAM1, EGR1, TXNRD1, MMP9, TLR4, MYC, SERPINE1, SERPINA1, HSPA1A, VEGF, CCL5, NFKB1, IL10, ADAM17, TNF, IL1R1, CASP3, or CD86 is measured such as to distinguish between an ovarian cancer diagnosed subject and a melanoma cancer diagnosed subject in a reference population; TIMP1, MAPK14, SSI3, PTPRC, or IL1RN is measured such as to distinguish between an ovarian cancer diagnosed subject and a breast cancer diagnosed subject in a reference population; LTA is measured such as to distinguish between an ovarian cancer diagnosed subject and a cervical cancer diagnosed subject in a reference population; or CASP3 or APAF1 is measured such as to distinguish between an ovarian cancer diagnosed subject and a lung cancer diagnosed subject in a reference population. Wherein the constituent is selected from the Human Cancer General Precision Profile™ (Table B), TIMP1, IL1B, or RB1 is measured such as to distinguish between an ovarian cancer diagnosed subject and a colon cancer diagnosed subject in a reference population; TGFB1, TIMP1, SERPINE1, NFKB1, RHOA, IL1B, IFITM1, EGR1, CDKN1A, ICAM1, SEMA4D, E2F1, MMP9, THBS1, BRAF, SRC, PLAU, TNFRSF1A, NOTCH2, NME4, FOS, PLAUR, MYC, or SOCS1 is measured such as to distinguish between an ovarian cancer diagnosed subject and a melanoma cancer diagnosed subject in a reference population; TIMP1, MMP9, CDKN1A, or IFITM1 is measured such as to distinguish between an ovarian cancer diagnosed subject and a breast cancer diagnosed subject in a reference population; or MYCL1 or AKT1 is measured such as to distinguish between an ovarian cancer diagnosed subject and a cervical cancer diagnosed subject in a reference population. Wherein the constituent is selected from the Precision Profile™ for EGR1 (Table C), ALOX5 or EP300 is measured such as to distinguish between an ovarian cancer diagnosed subject and a colon cancer diagnosed subject in a reference population; TGFB1, PDGFA, ALOX5, NFKB1, SERPINE1, EP300, ICAM1, CREBBP, EGR1, THBS1, SRC, PLAU, CEBPB, MAPK1, FOS, or CDKN2D is measured such as to distinguish between an ovarian cancer diagnosed subject and a melanoma cancer diagnosed subject in a reference population; or ALOX5 or EP300 is measured such as to distinguish between an ovarian cancer diagnosed subject and a breast cancer diagnosed subject in a reference population.

In yet a further aspect, wherein the constituent is selected from the Precision Profile™ for Inflammatory Response (Table A), IFI16, LTA, ADAM17, MAPK14, PTPRC, TLR4, TXNRD1, VEGF, TLR2, ELA2, GZMB, MNDA, TNFRSF1A, TIMP1, CD86, IL15, or HMOX1 is measured such as to distinguish between a prostate cancer diagnosed subject and a colon cancer diagnosed subject in a reference population; IFI16, MAPK14, ADAM17, TIMP1, LTA, TLR2, TNFRSF1A, SSI3, PTPRC, TXNRD1, TGFB1, TLR4, EGR1, MYC, MNDA, IL1R1, IL1RN, HMOX1, MMP9, VEGF, IL1B, PTGS2, ELA2, SERPINE1, CD86, TNF, IL15, or MHC2TA is measured such as to distinguish between a prostate cancer diagnosed subject and a melanoma cancer diagnosed subject in a reference population; or CCL5, EGR1, TGFB1, IL1RN, TIMP1, CCL3, TNF, PLAUR, IL1B, CXCR3, PTGS2, TNFRSF1A, PTPRC, NFKB1, ICAM1, CD8A, IRF1, IL32, HMOX1, SERPINA1, HSPA1A, or ALOX5 is measured such as to distinguish between a prostate cancer diagnosed subject and a lung cancer diagnosed subject in a reference population. Wherein the constituent is selected from the Human Cancer General Precision Profile™ (Table B), IL18, RB1 or ANGPT1 is measured such as to distinguish between a prostate cancer diagnosed subject and a colon cancer diagnosed subject in a reference population; BRAF, EGR1, RB1, SERPINE1, NFKB1, or RHOA is measured such as to distinguish between a prostate cancer diagnosed subject and a melanoma cancer diagnosed subject in a reference population; or EGR1, TGFB1, S100A4, RHOA, PLAUR, CDKN1A, TIMP1, WNT1, SEMA4D, E2F1, or SOCS1 is measured such as to distinguish between a prostate cancer diagnosed subject and a lung cancer diagnosed subject in a reference population. Wherein the constituent is selected from the Precision Profile™ for EGR1 (Table C), TOPBP1 is measured such as to distinguish between a prostate cancer diagnosed subject and a colon cancer diagnosed subject in a reference population; EP300, EGR1, MAPK1, ALOX5, PLAU, SERPINE1, or NFKB1 is measured such as to distinguish between a prostate cancer diagnosed subject and a melanoma cancer diagnosed subject in a reference population; or EGR1, TGFB1, S100A6, EP300, or CREBBP is measured such as to distinguish between a prostate cancer diagnosed subject and a lung cancer diagnosed subject in a reference population.

In another aspect, wherein the constituent is selected from the Precision Profile™ for Inflammatory Response (Table A), LTA, IFI16, PTPRC, CD86, ADAM17, HMOX1, TXNRD1, MYC, MHC2TA, MAPK14, TLR2, CD19, TNFRSF1A, TIMP1, TNF, IL23A, HLADRA, TLR4, PLAUR, PTGS2, PLA2G7, CCR5, or TOSO is measured such as to distinguish between a colon cancer diagnosed subject and a breast cancer diagnosed subject in a reference population; TGFB1, CCL5, SSI3, TIMP1, EGR1, IFI16, or SERPINE1 is measured such as to distinguish between a colon cancer diagnosed subject and a melanoma cancer diagnosed subject in a reference population; LTA, IFI16, PTPRC, TNFRSF1A, TIMP1, MNDA, TLR2, IL1RN, VEGF, MAPK14, TLR4, TXNRD1, SSI3, PLAUR, PTGS2, TGFB1, HMOX1, IL1B, IL10, CASP3, ADAM17, or SERPINA1 is measured such as to distinguish between a colon cancer diagnosed subject and an ovarian cancer diagnosed subject in a reference population; IFI16, LTA, TNFRSF1A, PTPRC, VEGF, TNF, TIMP1, CD86, PLAUR, PTGS2, ADAM17, MYC, TGFB1, IL1RN, HMOX1, TLR4, TLR2, MNDA, MAPK14, TXNRD1, ICAM1, CASP3, IL1B, CCL5, NFKB1, HLADRA, SSI3, SERPINA1, HSPA1A, MMP9, SERPINE1, MHC2TA, CXCR3, PLA2G7, CCR5, CD19, or EGR1 is measured such as to distinguish between a colon cancer diagnosed subject and a cervical cancer diagnosed subject in a reference population; LTA, CD86, IFI16, PTPRC, VEGF, ADAM17, TXNRD1, TNF, MNDA, TIMP1, HMOX1, PTGS2, TNFRSF1A, IL1RN, TLR4, MYC, IL10, MAPK14, TLR2, PLAUR, TGFB1, ELA2, PLA2G7, IL1R1, NFKB1, IL1B, IL18, CXCR3, IL15, CCL5, HLADRA, EGR1, HSPA1A, IL5, ICAM1, SSI3, or IL8 is measured such as to distinguish between a colon cancer diagnosed subject and a lung cancer diagnosed subject in a reference population; or IFI16, LTA, ADAM17, MAPK14, PTPRC, TLR4, TXNRD1, VEGF, TLR2, ELA2, GZMB, MNDA, TNFRSF1A, TIMP1, CD86, IL15, or HMOX1 is measured such as to distinguish between a colon cancer diagnosed subject and a prostate cancer diagnosed subject in a reference population. Wherein the constituent is selected from the Human Cancer General Precision Profile™ (Table B), EGR1, TGFB1, SERPINE1, E2F1, THBS1, IFITM1, or FGFR2 is measured such as to distinguish between a colon cancer diagnosed subject and a melanoma cancer diagnosed subject in a reference population; TIMP1, IL1B, or RB1 is measured such as to distinguish between a colon cancer diagnosed subject and an ovarian cancer diagnosed subject in a reference population; NME4, BRAF, NFKB1, SMAD4, ABL2, RHOA, NOTCH2, TIMP1, TGFB1, SEMA4D, BCL2, CDK2, NRAS, RB1, CDK5, IL1B, or FOS is measured such as to distinguish between a colon cancer diagnosed subject and a cervical cancer diagnosed subject in a reference population; BRAF, NME4, RB1, SMAD4, NFKB1, RHOA, BRCA1, APAF1, NRAS, PLAU, CDK5, VEGF, TIMP1, BCL2, RAF1, TGFB1, SEMA4D, CFLAR, NOTCH2, or ABL2 is measured such as to distinguish between a colon cancer diagnosed subject and a lung cancer diagnosed subject in a reference population; or IL18, RB1 or ANGPT1 is measured such as to distinguish between a colon cancer diagnosed subject and a prostate cancer diagnosed subject in a reference population. Wherein the constituent is selected from the Precision Profile™ for EGR1 (Table C), PDGFA, TGFB1, SERPINE1, EGR1, THBS1, SMAD3, or NFATC2 is measured such as to distinguish between a colon cancer diagnosed subject and a melanoma cancer diagnosed subject in a reference population; ALOX5 or EP300 is measured such as to distinguish between a colon cancer diagnosed subject and an ovarian cancer diagnosed subject in a reference population; EP300, ALOX5, MAPK1, CREBBP, NFKB1, ICAM1, SMAD3, TGFB1, CEBPB, TOPBP1, NR4A2, FOS, or EGR1 is measured such as to distinguish between a colon cancer diagnosed subject and a cervical cancer diagnosed subject in a reference population; EP300, TOPBP1, ALOX5, NFKB1, MAPK1, CREBBP, PLAU, SMAD3, NAB1, MAP2K1, TGFB1, RAF1, or EGR1 is measured such as to distinguish between a colon cancer diagnosed subject and a lung cancer diagnosed subject in a reference population; or TOPBP1 is measured such as to distinguish between a colon cancer diagnosed subject and a prostate cancer diagnosed subject in a reference population.

In a further aspect, wherein the constituent is selected from the Precision Profile™ for Inflammatory Response (Table A), IFI16, TIMP1, MAPK14, LTA, TGFB1, HMOX1, TNFRSF1A, PTPRC, PLAUR, EGR1, ADAM17, TLR2, MYC, SSI3, TNF, CD86, IL1B, CCL5, MHC2TA, CXCR3, TXNRD1, PTGS2, ICAM1, IL1RN, SERPINE1, CD4, NFKB1, CCR5, TLR4, IL18BP, CCL3, HLADRA, MMP9, or IL32 is measured such as to distinguish between a melanoma cancer diagnosed subject and a breast cancer diagnosed subject in a reference population; TGFB1, CCL5, SSI3, TIMP1, EGR1, IFI16, or SERPINE1 is measured such as to distinguish between a melanoma cancer diagnosed subject and a colon cancer diagnosed subject in a reference population; IFI16, MAPK14, TNFRSF1A, TIMP1, PTPRC, TGFB1, IL1B, SSI3, IL1RN, LTA, PLAUR, MNDA, HMOX1, TLR2, PTGS2, ICAM1, EGR1, TXNRD1, MMP9, TLR4, MYC, SERPINE1, SERPINA1, HSPA1A, VEGF, CCL5, NFKB1, IL10, ADAM17, TNF, IL1R1, CASP3, or CD86 is measured such as to distinguish between a melanoma cancer diagnosed subject and an ovarian cancer diagnosed subject in a reference population; IFI16, PLAUR, TGFB1, TNFRSF1A, LTA, TIMP1, MAPK14, ICAM1, IL1RN, PTPRC, IL1B, ADAM17, PTGS2, CCL5, TNF, EGR1, SSI3, HMOX1, MYC, CD86, IRF1, MNDA, TLR2, NFKB1, SERPINE1, HSPA1A, SERPINA1, TXNRD1, MMP9, VEGF, TLR4, CASP3, CXCR3, CD4, CCL3, CASP1, MHC2TA, CCR5, TNFSF5, HLADRA, IL18BP, IL1R1, or IL32 is measured such as to distinguish between a melanoma cancer diagnosed subject and a cervical cancer diagnosed subject in a reference population; IFI16, LTA, TIMP1, MAPK14, EGR1, ADAM17, PTPRC, HMOX1, CD86, TGFB1, CCL5, IL1RN, TNFRSF1A, TNF, PTGS2, IL1B, MNDA, PLAUR, TXNRD1, MYC, IL10, TLR2, SSI3, MMP9, VEGF, NFKB1, TLR4, ICAM1, SERPINE1, SERPINA1, HSPA1A, CXCR3, IL1R1, CCL3, IRF1, ELA2, CASP1, CCR5, CD4, IL18, MHC2TA, CXCL1, IL18BP, IL5, HLADRA, or TNFSF6 is measured such as to distinguish between a melanoma cancer diagnosed subject and a lung cancer diagnosed subject in a reference population; or IFI16, MAPK14, ADAM17, TIMP1, LTA, TLR2, TNFRSF1A, SSI3, PTPRC, TXNRD1, TGFB1, TLR4, EGR1, MYC, MNDA, IL1R1, IL1RN, HMOX1, MMP9, VEGF, IL1B, PTGS2, ELA2, SERPINE1, CD86, TNF, IL15, MHC2TA is measured such as to distinguish between a melanoma cancer diagnosed subject and a prostate cancer diagnosed subject in a reference population. Wherein the constituent is selected from the Human Cancer General Precision Profile™ (Table B), EGR1, TGFB1, NFKB1, SRC, TP53, ABL1, SERPINE1, or CDKN1A is measured such as to distinguish between a melanoma cancer diagnosed subject and a breast cancer diagnosed subject in a reference population; EGR1, TGFB1, SERPINE1, E2F1, THBS1, IFITM1, or FGFR2 is measured such as to distinguish between a melanoma cancer diagnosed subject and a colon cancer diagnosed subject in a reference population; TGFB1, TIMP1, SERPINE1, NFKB1, RHOA, IL1B, IFITM1, EGR1, CDKN1A, ICAM1, SEMA4D, E2F1, MMP9, THBS1, BRAF, SRC, PLAU, TNFRSF1A, NOTCH2, NME4, FOS, PLAUR, MYC, or SOCS1 is measured such as to distinguish between a melanoma cancer diagnosed subject and an ovarian cancer diagnosed subject in a reference population; EGR1, ICAM1, TGFB1, SERPINE1, NME4, NFKB1, SEMA4D, TIMP1, TNF, BRAF, NOTCH2, SRC, RHOA, IFITM1, FOS, CDKN1A, PLAUR, PLAU, TNFRSF1A, IL1B, E2F1, TP53, THBS1, MYC, ABL2, AKT1, MMP9, SOCS1, SMAD4, CDK5, CDK2, ABL1, RHOC, BRCA1, or BCL2 is measured such as to distinguish between a melanoma cancer diagnosed subject and a cervical cancer diagnosed subject in a reference population; EGR1, TGFB1, NFKB1, RHOA, BRAF, CDKN1A, TIMP1, TNF, PLAU, IFITM1, ICAM1, SEMA4D, THBS1, SERPINE1, NME4, NOTCH2, E2F1, SMAD4, MMP9, TP53, FOS, PLAUR, CDK5, IL1B, RB1, MYC, AKT1, SRC, TNFRSF1A, BRCA1, ABL2, PTCH1, CDK2, IGFBP3, CDC25A, SOCS1, WNT1, RHOC, PTEN, ITGB1, S100A4, ABL1, APAF1, VHL, or BCL2 is measured such as to distinguish between a melanoma cancer diagnosed subject and a lung cancer diagnosed subject in a reference population; or BRAF, EGR1, RB1, SERPINE1, NFKB1, or RHOA is measured such as to distinguish between a melanoma cancer diagnosed subject and a prostate cancer diagnosed subject in a reference population. Wherein the constituent is selected from the Precision Profile™ for EGR1 (Table C), TGFB1, EGR1, SMAD3, NFKB1, SRC, TP53, NFATC2, PDGFA, or SERPINE1 is measured such as to distinguish between a melanoma cancer diagnosed subject and a breast cancer diagnosed subject in a reference population; PDGFA, TGFB1, SERPINE1, EGR1, THBS1, SMAD3, or NFATC2 is measured such as to distinguish between a melanoma cancer diagnosed subject and a colon cancer diagnosed subject in a reference population; TGFB1, PDGFA, ALOX5, NFKB1, SERPINE1, EP300, ICAM1, CREBBP, EGR1, THBS1, SRC, PLAU, CEBPB, MAPK1, FOS, or CDKN2D is measured such as to distinguish between a melanoma cancer diagnosed subject and an ovarian cancer diagnosed subject in a reference population; EGR1, ICAM1, PDGFA, TGFB1, EP300, SERPINE1, CREBBP, ALOX5, NFKB1, MAPK1, SRC, SMAD3, FOS, PLAU, CEBPB, TP53, THBS1, MAP2K1, NFATC2, NR4A2, EGR2, EGR3, TOPBP1, or CDKN2D is measured such as to distinguish between a melanoma cancer diagnosed subject and a cervical cancer diagnosed subject in a reference population; EGR1, TGFB1, EP300, PDGFA, NFKB1, CREBBP, ALOX5, MAPK1, PLAU, SMAD3, ICAM1, THBS1, SERPINE1, MAP2K1, TP53, TOPBP1, FOS, NFATC2, SRC, CEBPB, CDKN2D, NR4A2, PTEN, EGR2, or EGR3 is measured such as to distinguish between a melanoma cancer diagnosed subject and a lung cancer diagnosed subject in a reference population; or EP300, EGR1, MAPK1, ALOX5, PLAU, SERPINE1, or NFKB1 is measured such as to distinguish between a melanoma cancer diagnosed subject and a prostate cancer diagnosed subject in a reference population.

Preferably, the constituents are selected so as to distinguish, e.g., classify between a subjects with different cancer types with at least 75%, 80%, 85%, 90%, 95%, 97%, 98%, 99% or greater accuracy. By “accuracy” is meant that the method has the ability to distinguish, e.g., classify, between subjects having breast cancer, ovarian cancer, cervical cancer, prostate cancer, lung cancer, colon cancer or melanoma. For example, the methods are capable of distinguishing between a subject having breast cancer and a subject having colon cancer, lung cancer, melanoma, cervical cancer or ovarian cancer. Accuracy is determined for example by comparing the results of the Gene Precision Profiling™ to standard accepted clinical methods of diagnosing the particular cancer type.

For example the combination of constituents are selected according to any of the models enumerated in Tables A1a, A2a, A3a, A4a, A5a, A6a, Ala, A8a, A9a, A10a, A11a, A12a, A13a, A14a, A15a, A16a, A17a, A18a, B1a, B2a, B3a, B4a, B5a, B6a, B7a, B8a, B9a, B10a, B11a, B12a, B13a, B14a, B15a, B16a, B17a, B18a, C1a, C2a, C3a, C4a, C5a, C6a, C7a, C8a, C9a, C10a, C11a, C12a, C13a, C14a, C15a, C16a, and C17a.

In some embodiments, the methods of the present invention are used in conjunction with standard accepted clinical methods to diagnose cancer.

The sample is any sample derived from a subject which contains RNA. For example, the sample is blood, a blood fraction, body fluid, a population of cells or tissue from the subject, a cervical cell, or a rare circulating tumor cell or circulating endothelial cell found in the blood.

Also included in the invention are kits for the detection of cancer in a subject, containing at least one reagent for the detection or quantification of any constituent measured according to the methods of the invention and instructions for using the kit.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a graphical representation of a 2-gene model for cancer based on disease-specific genes, capable of distinguishing between subjects afflicted with cancer and subjects in a reference population with a discrimination line overlaid onto the graph as an example of the Index Function evaluated at a particular logit value. Values above and to the left of the line represent subjects predicted to be in the reference population. Values below and to the right of the line represent subjects predicted to be in the cancer population. ALOX5 values are plotted along the Y-axis, S100A6 values are plotted along the X-axis.

FIG. 2 is a graphical representation of a 2-gene model, ALOX5, and PLAUR, based on the Precision Profile™ for Inflammation (Table A), capable of distinguishing between subjects afflicted with breast cancer and subjects afflicted with melanoma (active disease, all stages), with a discrimination line overlaid onto the graph as an example of the Index Function evaluated at a particular logit value. Values to the left of the line (“X”s) represent subjects predicted to be in the breast cancer population. Values to the right of the line (“O”s) represent subjects predicted to be in the melanoma population (active disease, all stages). ALOX5 values are plotted along the Y-axis. PLAUR values are plotted along the X-axis.

FIG. 3 is a graphical representation of a 2-gene model, IRF1, and MHC2TA, based on the Precision Profile™ for Inflammation (Table A), capable of distinguishing between subjects afflicted with breast cancer and subjects afflicted with ovarian cancer, with a discrimination line overlaid onto the graph as an example of the Index Function evaluated at a particular logit value. Values to the left of the line (“X”s) represent subjects predicted to be in the breast cancer population. Values to the right of the line (“O”s) represent subjects predicted to be in the ovarian cancer population. IRF1 values are plotted along the Y-axis. MHC2TA values are plotted along the X-axis.

FIG. 4 is a graphical representation of a 2-gene model, ELA2, and IRF1, based on the Precision Profile™ for Inflammation (Table A), capable of distinguishing between subjects afflicted with breast cancer and subjects afflicted with cervical cancer, with a discrimination line overlaid onto the graph as an example of the Index Function evaluated at a particular logit value. Values to the right of the line (“X”s) represent subjects predicted to be in the breast cancer population. Values to the left of the line (“O”s) represent subjects predicted to be in the cervical cancer population. ELA2 values are plotted along the Y-axis. IRF1 values are plotted along the X-axis.

FIG. 5 is a graphical representation of a 2-gene model, IFI16, and LTA, based on the Precision Profile™ for Inflammation (Table A), capable of distinguishing between subjects afflicted with cervical cancer and subjects afflicted with colon cancer, with discrimination lines overlaid onto the graph as an example of the Index Function evaluated at a particular logit value. Values in the bottom left quadrant (“X”s) represent subjects predicted to be in the cervical cancer population. Values in the upper right quadrant (“O”s) represent subjects predicted to be in the colon cancer population. IFI16 values are plotted along the Y-axis. LTA values are plotted along the X-axis.

FIG. 6 is a graphical representation of a 2-gene model, IFI16, and PLAUR, based on the Precision Profile™ for Inflammation (Table A), capable of distinguishing between subjects afflicted with cervical cancer and subjects afflicted with melanoma (active disease, all stages), with discrimination lines overlaid onto the graph as an example of the Index Function evaluated at a particular logit value. Values in the bottom left quadrant (“X”s) represent subjects predicted to be in the cervical cancer population. Values in the upper right quadrant (“O”s) represent subjects predicted to be in the melanoma population (active disease, all stages). IFI16 values are plotted along the Y-axis. PLAUR values are plotted along the X-axis.

FIG. 7 is a graphical representation of a 2-gene model, MIF, and TGFB1, based on the Precision Profile™ for Inflammation (Table A), capable of distinguishing between subjects afflicted with colon cancer and subjects afflicted with melanoma (active disease, all stages), with a discrimination line overlaid onto the graph as an example of the Index Function evaluated at a particular logit value. Values to the left of the line (“X”s) represent subjects predicted to be in the colon cancer population. Values to the right of the line (“O”s) represent subjects predicted to be in the melanoma population (active disease, all stages). MIF values are plotted along the Y-axis. TGFB1 values are plotted along the X-axis.

FIG. 8 is a graphical representation of a 2-gene model, APAF1, and ELA2, based on the Precision Profile™ for Inflammation (Table A), capable of distinguishing between subjects afflicted with breast cancer and subjects afflicted with lung cancer, with a discrimination line overlaid onto the graph as an example of the Index Function evaluated at a particular logit value. Values to the right of the line (“X”s) represent subjects predicted to be in the breast cancer population. Values to the left of the line (“O”s) represent subjects predicted to be in the lung cancer population. APAF1 values are plotted along the Y-axis. ELA2 values are plotted along the X-axis.

FIG. 9 is a graphical representation of a 2-gene model, ICAM1, and TXNRD1, based on the Precision Profile™ for Inflammation (Table A), capable of distinguishing between subjects afflicted with cervical cancer and subjects afflicted with lung cancer, with a discrimination line overlaid onto the graph as an example of the Index Function evaluated at a particular logit value. Values to the right of the line (“X”s) represent subjects predicted to be in the cervical cancer population. Values to the left of the line (“O”s) represent subjects predicted to be in the lung cancer population. ICAM1 values are plotted along the Y-axis. TXNRD1 values are plotted along the X-axis.

FIG. 10 is a graphical representation of a 2-gene model, ALOX5, and TNFRSF1A, based on the Precision Profile™ for Inflammation (Table A), capable of distinguishing between subjects afflicted with colon cancer and subjects afflicted with lung cancer, with a discrimination line overlaid onto the graph as an example of the Index Function evaluated at a particular logit value. Values to the right of the line (“X”s) represent subjects predicted to be in the colon cancer population. Values to the left of the line (“O”s) represent subjects predicted to be in the lung cancer population. ALOX5 values are plotted along the Y-axis. TNFRSF1A values are plotted along the X-axis.

FIG. 11 is a graphical representation of a 2-gene model, APAF1, and TNXRD1, based on the Precision Profile™ for Inflammation (Table A), capable of distinguishing between subjects afflicted with lung cancer and subjects afflicted with melanoma (active disease, all stages), with a discrimination line overlaid onto the graph as an example of the Index Function evaluated at a particular logit value. Values to the left of the line (“X”s) represent subjects predicted to be in the lung cancer population. Values to the right of the line (“O”s) represent subjects predicted to be in the melanoma population (active disease, all stages). APAF1 values are plotted along the Y-axis. TNXRD1 values are plotted along the X-axis.

FIG. 12 is a graphical representation of a 2-gene model, CCL5, and EGR1, based on the Precision Profile™ for Inflammation (Table A), capable of distinguishing between subjects afflicted with lung cancer and subjects afflicted with prostate cancer, with a discrimination line overlaid onto the graph as an example of the Index Function evaluated at a particular logit value. Values to the left of the line (“X”s) represent subjects predicted to be in the lung cancer population. Values to the right of the line (“O”s) represent subjects predicted to be in the prostate cancer population. CCL5 values are plotted along the Y-axis. EGR1 values are plotted along the X-axis.

FIG. 13 is a graphical representation of a 2-gene model, ALOX5, and MAPK14, based on the Precision Profile™ for Inflammation (Table A), capable of distinguishing between subjects afflicted with colon cancer and subjects afflicted with ovarian cancer, with a discrimination line overlaid onto the graph as an example of the Index Function evaluated at a particular logit value. Values to the right of the line (“X”s) represent subjects predicted to be in the colon cancer population. Values to the left of the line (“O”s) represent subjects predicted to be in the ovarian cancer population. ALOX5 values are plotted along the Y-axis. MAPK14 values are plotted along the X-axis.

FIG. 14 is a graphical representation of a 2-gene model, IFI16, and MAPK14, based on the Precision Profile™ for Inflammation (Table A), capable of distinguishing between subjects afflicted with melanoma (active disease, all stages) and subjects afflicted with ovarian cancer, with discrimination lines overlaid onto the graph as an example of the Index Function evaluated at a particular logit value. Values in the upper right quadrant (“X”s) represent subjects predicted to be in the melanoma population (active disease, all stages). Values in the bottom left quadrant (“O”s) represent subjects predicted to be in the ovarian cancer population. IFI16 values are plotted along the Y-axis. MAPK14 values are plotted along the X-axis.

FIG. 15 is a graphical representation of a 2-gene model, CCR5, and LTA, based on the Precision Profile™ for Inflammation (Table A), capable of distinguishing between subjects afflicted with colon cancer and subjects afflicted with prostate cancer, with a discrimination line overlaid onto the graph as an example of the Index Function evaluated at a particular logit value. Values to the right of the line (“X”s) represent subjects predicted to be in the colon cancer population. Values to the left of the line (“O”s) represent subjects predicted to be in the prostate cancer population. CCR5 values are plotted along the Y-axis. LTA values are plotted along the X-axis.

FIG. 16 is a graphical representation of a 2-gene model, APAF1, and TNFRSF1A, based on the Precision Profile™ for Inflammation (Table A), capable of distinguishing between subjects afflicted with melanoma (active disease, all stages) and subjects afflicted with prostate cancer, with a discrimination line overlaid onto the graph as an example of the Index Function evaluated at a particular logit value. Values to the right of the line (“X”s) represent subjects predicted to be in the melanoma population (active disease, all stages). Values to the left of the line (“O”s) represent subjects predicted to be in the prostate cancer population. APAF1 values are plotted along the Y-axis. TNFRSF1A values are plotted along the X-axis.

FIG. 17 is a graphical representation of a 2-gene model, ALOX5, and TNFRSF1A, based on the Precision Profile™ for Inflammation (Table A), capable of distinguishing between subjects afflicted with breast cancer and subjects afflicted with colon cancer, with a discrimination line overlaid onto the graph as an example of the Index Function evaluated at a particular logit value. Values to the left of the line (“X”s) represent subjects predicted to be in the breast cancer population. Values to the right of the line (“O”s) represent subjects predicted to be in the colon cancer population. ALOX5 values are plotted along the Y-axis. TNFRSF1A values are plotted along the X-axis.

FIG. 18 is a graphical representation of a 2-gene model, RAF1 and TGFB1, based on the Human Cancer General Precision Profile™ (Table B), capable of distinguishing between subjects afflicted with breast cancer and subjects afflicted with melanoma (active disease, stages 2-4), with a discrimination line overlaid onto the graph as an example of the Index Function evaluated at a particular logit value. Values to the left of the line (“X”s) represent subjects predicted to be in the breast cancer population. Values to the right of the line (“O”s) represent subjects predicted to be in the melanoma population (active disease, stages 2-4). RAF1 values are plotted along the Y-axis, TGFB1 values are plotted along the X-axis.

FIG. 19 is a graphical representation of a 2-gene model, MYCL1 and TIMP1, based on the Human Cancer General Precision Profile™ (Table B), capable of distinguishing between subjects afflicted with breast cancer and subjects afflicted with ovarian cancer, with a discrimination line overlaid onto the graph as an example of the Index Function evaluated at a particular logit value. Values to the right of the line (“X”s) represent subjects predicted to be in the breast cancer population. Values to the left of the line (“O”s) represent subjects predicted to be in the ovarian cancer population. MYCL1 values are plotted along the Y-axis, TIMP1 values are plotted along the X-axis.

FIG. 20 is a graphical representation of a 2-gene model, HRAS and SMAD4, based on the Human Cancer General Precision Profile™ (Table B), capable of distinguishing between subjects afflicted with breast cancer and subjects afflicted with cervical cancer, with a discrimination line overlaid onto the graph as an example of the Index Function evaluated at a particular logit value. Values to the right of the line (“X”s) represent subjects predicted to be in the breast cancer population. Values to the left of the line (“O”s) represent subjects predicted to be in the cervical cancer population. HRAS values are plotted along the Y-axis, SMAD4 values are plotted along the X-axis.

FIG. 21 is a graphical representation of a 2-gene model, BRAF and NME4 based on the Human Cancer General Precision Profile™ (Table B), capable of distinguishing between subjects afflicted with cervical cancer and subjects afflicted with colon cancer, with a discrimination line overlaid onto the graph as an example of the Index Function evaluated at a particular logit value. Values to the left of the line (“X”s) represent subjects predicted to be in the cervical cancer population. Values to the right of the line (“O”s) represent subjects predicted to be in the colon cancer population. BRAF values are plotted along the Y-axis, NME4 values are plotted along the X-axis.

FIG. 22 is a graphical representation of a 2-gene model, RAF1 and TGFB1, based on the Human Cancer General Precision Profile™ (Table B), capable of distinguishing between subjects afflicted with cervical cancer and subjects afflicted with melanoma (active disease, stages 2-4), with a discrimination line overlaid onto the graph as an example of the Index Function evaluated at a particular logit value. Values to the left of the line (“X”s) represent subjects predicted to be in the cervical cancer population. Values to the right of the line (“O”s) represent subjects predicted to be in the melanoma population (active disease, stages 2-4). RAF1 values are plotted along the Y-axis, TGFB1 values are plotted along the X-axis.

FIG. 23 is a graphical representation of a 2-gene model, ATM and TP53, based on the Human Cancer General Precision Profile™ (Table B), capable of distinguishing between subjects afflicted with colon cancer and subjects afflicted with melanoma (active disease, stages 2-4), with a discrimination line overlaid onto the graph as an example of the Index Function evaluated at a particular logit value. Values above and to the left of the line (“X”s) represent subjects predicted to be in the colon cancer population. Values below and to the right of the line (“O”s) represent subjects predicted to be in the melanoma population (active disease, stages 2-4). ATM values are plotted along the Y-axis, TP53 values are plotted along the X-axis.

FIG. 24 is a graphical representation of a 2-gene model, RB1 and TNFRSF10A, based on the Human Cancer General Precision Profile™ (Table B), capable of distinguishing between subjects afflicted with breast cancer and subjects afflicted with lung cancer, with a discrimination line overlaid onto the graph as an example of the Index Function evaluated at a particular logit value. Values above and to the left of the line (“X”s) represent subjects predicted to be in the breast cancer population. Values below and to the right of the line (“O”s) represent subjects predicted to be in the lung cancer population. RB1 values are plotted along the Y-axis, TNFRSF10A values are plotted along the X-axis.

FIG. 25 is a graphical representation of a 2-gene model, APAF1 and NME4, based on the Human Cancer General Precision Profile™ (Table B), capable of distinguishing between subjects afflicted with colon cancer and subjects afflicted with lung cancer, with a discrimination line overlaid onto the graph as an example of the Index Function evaluated at a particular logit value. Values to the right of the line (“X”s) represent subjects predicted to be in the colon cancer population. Values to the left of the line (“O”s) represent subjects predicted to be in the lung cancer population. APAF1 values are plotted along the Y-axis, NME4 values are plotted along the X-axis.

FIG. 26 is a graphical representation of a 2-gene model, EGR1 and THBS1, based on the Human Cancer General Precision Profile™ (Table B), capable of distinguishing between subjects afflicted with lung cancer and subjects afflicted with melanoma (active disease, stages 2-4) with a discrimination line overlaid onto the graph as an example of the Index Function evaluated at a particular logit value. Values below and to the left of the line (“X”s) represent subjects predicted to be in the lung cancer population. Values above and to the right of the line (“O”s) represent subjects predicted to be in the melanoma population (active disease, stages 2-4). EGR1 values are plotted along the Y-axis, THBS1 values are plotted along the X-axis.

FIG. 27 is a graphical representation of a 2-gene model, CFLAR and IL18, based on the Human Cancer General Precision Profile™ (Table B), capable of distinguishing between subjects afflicted with lung cancer and subjects afflicted with ovarian cancer, with a discrimination line overlaid onto the graph as an example of the Index Function evaluated at a particular logit value. Values to the left of the line (“X”s) represent subjects predicted to be in the lung cancer population. Values to the right of the line (“O”s) represent subjects predicted to be in the ovarian cancer population. CFLAR values are plotted along the Y-axis, IL18 values are plotted along the X-axis.

FIG. 28 is a graphical representation of a 2-gene model, EGR1 and TGFB1, based on the Human Cancer General Precision Profile™ (Table B), capable of distinguishing between subjects afflicted with lung cancer and subjects afflicted with prostate cancer, with a discrimination line overlaid onto the graph as an example of the Index Function evaluated at a particular logit value. Values below and to the right of the line (“X”s) represent subjects predicted to be in the lung cancer population. Values above and to the left of the line (“O”s) represent subjects predicted to be in the prostate cancer population. EGR1 values are plotted along the Y-axis, TGFB1 values are plotted along the X-axis.

FIG. 29 is a graphical representation of a 2-gene model, CFLAR and NME4 based on the Human Cancer General Precision Profile™ (Table B), capable of distinguishing between subjects afflicted with colon cancer and subjects afflicted with ovarian cancer, with a discrimination line overlaid onto the graph as an example of the Index Function evaluated at a particular logit value. Values above and to the right of the line (“X”s) represent subjects predicted to be in the colon cancer population. Values to below and to the left of the line (“O”s) represent subjects predicted to be in the ovarian cancer population. CFLAR values are plotted along the Y-axis, NME4 values are plotted along the X-axis.

FIG. 30 is a graphical representation of a 2-gene model, RAF1 and TGFB1, based on the Human Cancer General Precision Profile™ (Table B), capable of distinguishing between subjects afflicted with melanoma (active disease, stages 2-4) and subjects afflicted with ovarian cancer, with a discrimination line overlaid onto the graph as an example of the Index Function evaluated at a particular logit value. Values to the right of the line (“X”s) represent subjects predicted to be in the melanoma population (active disease, stages 2-4). Values to the left of the line (“O”s) represent subjects predicted to be in the ovarian cancer population. RAF1 values are plotted along the Y-axis, TGFB1 values are plotted along the X-axis.

FIG. 31 is a graphical representation of a 2-gene model, PLAUR and RB1, based on the Human Cancer General Precision Profile™ (Table B), capable of distinguishing between subjects afflicted with colon cancer and subjects afflicted with prostate cancer, with a discrimination line overlaid onto the graph as an example of the Index Function evaluated at a particular logit value. Values to the right of the line (“X”s) represent subjects predicted to be in the colon cancer population. Values to the left of the line (“O”s) represent subjects predicted to be in the prostate cancer population. PLAUR values are plotted along the Y-axis, RB1 values are plotted along the X-axis.

FIG. 32 is a graphical representation of a 2-gene model, BAD and RB1, based on the Human Cancer General Precision Profile™ (Table B), capable of distinguishing between subjects afflicted with melanoma (active disease, stages 2-4) and subjects afflicted with prostate cancer, with a discrimination line overlaid onto the graph as an example of the Index Function evaluated at a particular logit value. Values to the right of the line (“X”s) represent subjects predicted to be in the melanoma population (active disease, stages 2-4). Values to the left of the line (“O”s) represent subjects predicted to be in the prostate cancer population. BAD values are plotted along the Y-axis, RB1 values are plotted along the X-axis.

FIG. 33 is a graphical representation of a 2-gene model, RAF1 and TGFB1, based on the Precision Profile™ for EGR1 (Table C), capable of distinguishing between subjects afflicted with breast cancer and subjects afflicted with melanoma (active disease, stages 2-4), with a discrimination line overlaid onto the graph as an example of the Index Function evaluated at a particular logit value. Values to the left of the line (“X”s) represent subjects predicted to be in the breast cancer population. Values to the right the line (“Os”) represent subjects predicted to be in the melanoma population (active disease, stages 2-4). RAF1 values are plotted along the Y-axis, TGFB1 values are plotted along the X-axis.

FIG. 34 is a graphical representation of a 2-gene model, NAB2 and PLAU, based on the Precision Profile™ for EGR1 (Table C), capable of distinguishing between subjects afflicted with breast cancer and subjects afflicted with ovarian cancer, with a discrimination line overlaid onto the graph as an example of the Index Function evaluated at a particular logit value. Values below and to the right of the line (“X”s) represent subjects predicted to be in the breast cancer population. Values above and to the left of the line (“Os”) represent subjects predicted to be in the ovarian cancer population. NAB2 values are plotted along the Y-axis, PLAU values are plotted along the X-axis.

FIG. 35 is a graphical representation of a 2-gene model, EP300 and MAP2K1, based on the Precision Profile™ for EGR1 (Table C), capable of distinguishing between subjects afflicted with breast cancer and subjects afflicted with cervical cancer, with a discrimination line overlaid onto the graph as an example of the Index Function evaluated at a particular logit value. Values above the line (“X”s) represent subjects predicted to be in the breast cancer population. Values below the line (“Os”) represent subjects predicted to be in the cervical cancer population. EP300 values are plotted along the Y-axis, MAP2K1 values are plotted along the X-axis.

FIG. 36 is a graphical representation of a 2-gene model, ALOX5 and S100A6, based on the Precision Profile™ for EGR1 (Table C), capable of distinguishing between subjects afflicted with cervical cancer and subjects afflicted with colon cancer, with a discrimination line overlaid onto the graph as an example of the Index Function evaluated at a particular logit value. Values below the line (“X”s) represent subjects predicted to be in the cervical cancer population. Values above the line (“Os”) represent subjects predicted to be in the colon cancer population. ALOX5 values are plotted along the Y-axis, S100A6 values are plotted along the X-axis.

FIG. 37 is a graphical representation of a 2-gene model, RAF1 and TGFB1, based on the Precision Profile™ for EGR1 (Table C), capable of distinguishing between subjects afflicted with cervical cancer and subjects afflicted with melanoma (active disease, stages 2-4), with a discrimination line overlaid onto the graph as an example of the Index Function evaluated at a particular logit value. Values to the left of the line (“X”s) represent subjects predicted to be in the cervical cancer population. Values to the right the line (“Os”) represent subjects predicted to be in the melanoma population (active disease, stages 2-4). RAF1 values are plotted along the Y-axis, TGFB1 values are plotted along the X-axis.

FIG. 38 is a graphical representation of a 2-gene model, RAF1 and TGFB1, based on the Precision Profile™ for EGR1 (Table C), capable of distinguishing between subjects afflicted with colon cancer and subjects afflicted with melanoma (active disease, stages 2-4), with a discrimination line overlaid onto the graph as an example of the Index Function evaluated at a particular logit value. Values to the left of the line (“X”s) represent subjects predicted to be in the colon cancer population. Values to the right the line (“Os”) represent subjects predicted to be in the melanoma population (active disease, stages 2-4). RAF1 values are plotted along the Y-axis, TGFB1 values are plotted along the X-axis.

FIG. 39 is a graphical representation of a 2-gene model, NAB2 and TOPBP1, based on the Precision Profile™ for EGR1 (Table C), capable of distinguishing between subjects afflicted with breast cancer and subjects afflicted with lung cancer, with a discrimination line overlaid onto the graph as an example of the Index Function evaluated at a particular logit value. Values to the right of the line (“X”s) represent subjects predicted to be in the breast cancer population. Values to the left the line (“Os”) represent subjects predicted to be in the lung cancer population. NAB2 values are plotted along the Y-axis, TOPBP1 values are plotted along the X-axis.

FIG. 40 is a graphical representation of a 2-gene model, EP300 and FOS, based on the Precision Profile™ for EGR1 (Table C), capable of distinguishing between subjects afflicted with colon cancer and subjects afflicted with lung cancer, with a discrimination line overlaid onto the graph as an example of the Index Function evaluated at a particular logit value. Values above and to the left of the line (“X”s) represent subjects predicted to be in the colon cancer population. Values below and to the right the line (“Os”) represent subjects predicted to be in the lung cancer population. EP300 values are plotted along the Y-axis, FOS values are plotted along the X-axis.

FIG. 41 is a graphical representation of a 2-gene model, EGR1 and PDGFA, based on the Precision Profile™ for EGR1 (Table C), capable of distinguishing between subjects afflicted with lung cancer and subjects afflicted with melanoma (active disease, stages 2-4), with a discrimination line overlaid onto the graph as an example of the Index Function evaluated at a particular logit value. Values below and to the left of the line (“X”s) represent subjects predicted to be in the lung cancer population. Values above and to the right the line (“Os”) represent subjects predicted to be in the melanoma population (active disease, stages 2-4). EGR1 values are plotted along the Y-axis, PDGFA values are plotted along the X-axis.

FIG. 42 is a graphical representation of a 2-gene model, EGR1 and S100A6, based on the Precision Profile™ for EGR1 (Table C), capable of distinguishing between subjects afflicted with lung cancer and subjects afflicted with prostate cancer, with a discrimination line overlaid onto the graph as an example of the Index Function evaluated at a particular logit value. Values below and to the left of the line (“X”s) represent subjects predicted to be in the lung cancer population. Values above and to the right the line (“Os”) represent subjects predicted to be in the prostate cancer population. EGR1 values are plotted along the Y-axis, S100A6 values are plotted along the X-axis.

FIG. 43 is a graphical representation of a 2-gene model, RAF1 and TGFB1, based on the Precision Profile™ for EGR1 (Table C), capable of distinguishing between subjects afflicted with melanoma (active disease, stages 2-4) and subjects afflicted with ovarian cancer, with a discrimination line overlaid onto the graph as an example of the Index Function evaluated at a particular logit value. Values to the right of the line (“X”s) represent subjects predicted to be in the melanoma population (active disease, stages 2-4). Values to the left the line (“Os”) represent subjects predicted to be in the ovarian cancer population. RAF1 values are plotted along the Y-axis, TGFB1 values are plotted along the X-axis.

FIG. 44 is a graphical representation of a 2-gene model, MAP2K1 and TOPBP1, based on the Precision Profile™ for EGR1 (Table C), capable of distinguishing between subjects afflicted with colon cancer and subjects afflicted with prostate cancer, with a discrimination line overlaid onto the graph as an example of the Index Function evaluated at a particular logit value. Values to the right of the line (“X”s) represent subjects predicted to be in the colon cancer population. Values to the left the line (“Os”) represent subjects predicted to be in the prostate cancer population. MAP2K1 values are plotted along the Y-axis, TOPBP1 values are plotted along the X-axis.

FIG. 45 is a graphical representation of a 2-gene model, S100A6 and TGFB1, based on the Precision Profile™ for EGR1 (Table C), capable of distinguishing between subjects afflicted with prostate cancer and subjects afflicted with melanoma (active disease, stages 2-4), with a discrimination line overlaid onto the graph as an example of the Index Function evaluated at a particular logit value. Values above and to the left of the line (“X”s) represent subjects predicted to be in the prostate cancer population. Values below and to the right the line (“Os”) represent subjects predicted to be in the melanoma population (active disease, stages 2-4). S100A6 values are plotted along the Y-axis, TGFB1 values are plotted along the X-axis.

### DETAILED DESCRIPTION

### Definitions

The following terms shall have the meanings indicated unless the context otherwise requires:

“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Algorithm” is a set of rules for describing a biological condition. The rule set may be defined exclusively algebraically but may also include alternative or multiple decision points requiring domain-specific knowledge, expert interpretation or other clinical indicators.

An “agent” is a “composition” or a “stimulus”, as those terms are defined herein, or a combination of a composition and a stimulus.

“Amplification” in the context of a quantitative RT-PCR assay is a function of the number of DNA replications that are required to provide a quantitative determination of its concentration. “Amplification” here refers to a degree of sensitivity and specificity of a quantitative assay technique. Accordingly, amplification provides a measurement of concentrations of constituents that is evaluated under conditions wherein the efficiency of amplification and therefore the degree of sensitivity and reproducibility for measuring all constituents is substantially similar.

A “baseline profile data set” is a set of values associated with constituents of a Gene Expression Panel (Precision Profile™) resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

A “biological condition” of a subject is the condition of the subject in a pertinent realm that is under observation, and such realm may include any aspect of the subject capable of being monitored for change in condition, such as health; disease including cancer; trauma; aging; infection; tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

“Body fluid” of a subject includes blood, urine, spinal fluid, lymph, mucosal secretions, prostatic fluid, semen, haemolymph or any other body fluid known in the art for a subject.

“Breast Cancer” is a cancer of the breast tissue which can occur in both women and men. Types of breast cancer include ductal carcinoma (infiltrating ductal carcinoma (IDC), and ductal carcinoma in situ (DCIS), lobular carcinoma, inflammatory breast cancer, medullary carcinoma, colloid carcinoma, papillary carcinoma, and metaplastic carcinoma. As defined herein the term “breast cancer” also includes stage 1, stage 2, stage 3, and stage 4 breast cancer, estrogen-positive breast cancer, estrogen-negative breast cancer, Her2+ breast cancer, and Her2− breast cancer.

“Calibrated profile data set” is a function of a member of a first profile data set and a corresponding member of a baseline profile data set for a given constituent in a panel.

“Cervical Cancer” is a malignancy of the cervix. Types of malignant cervical tumors include squamous cell carcinoma, adenocarcinoma, adenosquamous carcinoma, small cell carcinoma, neuroendocrine carcinoma, melanoma, and lymphoma. As defined herein, the term “cervical cancer” includes Stage 1, Stage II, Stage III and Stage 1V cervical cancer, as defined by the TNM staging system.

A “circulating endothelial cell” (“CEC”) is an endothelial cell from the inner wall of blood vessels which sheds into the bloodstream under certain circumstances, including inflammation, and contributes to the formation of new vasculature associated with cancer pathogenesis. CECs may be useful as a marker of tumor progression and/or response to antiangiogenic therapy.

A “circulating tumor cell” (“CTC”) is a tumor cell of epithelial origin which is shed from the primary tumor upon metastasis, and enters the circulation. The number of circulating tumor cells in peripheral blood is associated with prognosis in patients with metastatic cancer. These cells can be separated and quantified using immunologic methods that detect epithelial cells.

A “clinical indicator” is any physiological datum used alone or in conjunction with other data in evaluating the physiological condition of a collection of cells or of an organism. This term includes pre-clinical indicators.

“Clinical parameters” encompasses all non-sample or non-Precision Profiles™ of a subject's health status or other characteristics, such as, without limitation, age (AGE), ethnicity (RACE), gender (SEX), and family history of cancer.

A “composition” includes a chemical compound, a nutraceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.

“Colorectal cancer” is a type of cancer that develops in the colon, or the rectum and includes adenocarcinomas, carcinoid tumors, gastrointestinal stromal tumors, and lymphomas of the digestive system. The term colorectal cancer encompasses both colon cancer and rectal cancer. The terms colorectal cancer and colon cancer are used interchangeably herein. As defined herein, the term “colorectal cancer” includes Stage 1, Stage 2, Stage 3, and Stage 4 colorectal cancer as determined by the Tumor/Nodes/Metastases (“TNM”) system which takes into account the size of the tumor, the number of involved lymph nodes, and the presence of any other metastases in conjuction with the AJCC stage groupings; and Stages A, B, C, and D, as determined by the Duke's classification system.

To “derive” a profile data set from a sample includes determining a set of values associated with constituents of a Gene Expression Panel (Precision Profile™) either (i) by direct measurement of such constituents in a biological sample.

“Distinct RNA or protein constituent” in a panel of constituents is a distinct expressed product of a gene, whether RNA or protein. An “expression” product of a gene includes the gene product whether RNA or protein resulting from translation of the messenger RNA.

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, statistical technique, or comparison, that takes one or more continuous or categorical inputs (herein called “parameters”) and calculates an output value, sometimes referred to as an “index” or “index value.” Non-limiting examples of “formulas” include comparisons to reference values or profiles, sums, ratios, and regression operators, such as coefficients or exponents, value transformations and normalizations (including, without limitation, those normalization schemes based on clinical parameters, such as gender, age, or ethnicity), rules and guidelines, statistical classification models, and neural networks trained on historical populations. Of particular use in combining constituents of a Gene Expression Panel (Precision Profile™) are linear and non-linear equations and statistical significance and classification analyses to determine the relationship between levels of constituents of a Gene Expression Panel (Precision Profile™) detected in a subject sample and the subject's risk of cancer. In panel and combination construction, of particular interest are structural and synactic statistical classification algorithms, and methods of risk index construction, utilizing pattern recognition features, including, without limitation, such established techniques such as cross-correlation, Principal Components Analysis (PCA), factor rotation, Logistic Regression Analysis (LogReg), Kolmogorov Smirnoff tests (KS), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques (CART, LART, LARTree, FlexTree, amongst others), Shrunken Centroids (SC), StepAIC, K-means, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a consituentes of a Gene Expression Panel (Precision Profile™) selection technique, such as forward selection, backwards selection, or stepwise selection, complete enumeration of all potential panels of a given size, genetic algorithms, voting and committee methods, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other clinical studies, or cross-validated within the study they were originally trained in, using such techniques as Bootstrap, Leave-One-Out (LOO) and 10-Fold cross-validation (10-Fold CV). At various steps, false discovery rates (FDR) may be estimated by value permutation according to techniques known in the art.

A “Gene Expression Panel” (Precision Profile™) is an experimentally verified set of constituents, each constituent being a distinct expressed product of a gene, whether RNA or protein, wherein constituents of the set are selected so that their measurement provides a measurement of a targeted biological condition.

A “Gene Expression Profile” is a set of values associated with constituents of a Gene Expression Panel (Precision Profile™) resulting from evaluation of a biological sample (or population or set of samples).

A “Gene Expression Profile Inflammation Index” is the value of an index function that provides a mapping from an instance of a Gene Expression Profile into a single-valued measure of inflammatory condition.

A Gene Expression Profile Cancer Index” is the value of an index function that provides a mapping from an instance of a Gene Expression Profile into a single-valued measure of a cancerous condition.

The “health” of a subject includes mental, emotional, physical, spiritual, allopathic, naturopathic and homeopathic condition of the subject.

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

“Inflammation” is used herein in the general medical sense of the word and may be an acute or chronic; simple or suppurative; localized or disseminated; cellular and tissue response initiated or sustained by any number of chemical, physical or biological agents or combination of agents.

“Inflammatory state” is used to indicate the relative biological condition of a subject resulting from inflammation, or characterizing the degree of inflammation.

A “large number” of data sets based on a common panel of genes is a number of data sets sufficiently large to permit a statistically significant conclusion to be drawn with respect to an instance of a data set based on the same panel.

“Lung cancer” is the growth of abnormal cells in the lungs, capable of invading and destroying other lung cells, and includes Stage 1, Stage 2 and Stage 3 lung cancer, small cell lung cancer, non-small cell lung cancer (squamous cell carcinoma, adenocarcinoma (e.g., bronchioloalveolar carcinoma and large-cell undifferentiated carcinoma), carcinoid tumors (typical and atypical), lymphomas of the lung, adenoid cystic carcinomas, hamartomas, lymphomas, sarcomas, and mesothelia.

“Melanoma” is a type of skin cancer which develops from melanocytes, the skin cells in the epidermis which produce the skin pigment melanin. As defined herein, the term “melanoma” includes Stage 1, Stage 2, Stage 3, and Stage 4 melanoma as determined by the Tumor/Nodes/Metastases (“TNM”) system which takes into account the size of the tumor, the number of involved lymph nodes, and the presence of any other metastases. As used herein, melanoma includes melanoma, non-melanotic melanoma, nodular melanoma, acral lentiginous melanoma, and lentigo maligna. “Active melanoma” indicates a subject having melanoma with clinical evidence of disease, and includes subjects that have had blood drawn within 2-3 weeks post resection, although no clinical evidence of disease may be present after resection. “Inactive melanoma” indicates subjects having no clinical evidence of disease.

“Non-melanoma” is a type of skin cancer which develops from skin cells other than melanocytes, and includes basal cell carcinoma, squamous cell carcinoma, cutaneous T-cell lymphoma, Merkel cell carcinoma, dermatofibrosarcoma protuberans, and Paget's disease.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating the Predictive Value of a Diagnostic Test, How to Prevent Misleading or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation of Laboratory Procedures,” chapter 14 in Teitz, Fundamentals of Clinical Chemistry, Burns and Ashwood (eds.), 4^{th} edition 1996, W.B. Saunders Company, pages 192-199; and Zweig et al., “ROC Curve Analysis: An Example Showing the Relationships Among Serum Lipid and Apolipoprotein Concentrations in Identifying Subjects with Coronory Artery Disease,” Clin. Chem., 1992, 38(8): 1425-1428. An alternative approach using likelihood functions, BIC, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

A “normal” subject is a subject who is generally in good health, has not been diagnosed with cancer, is asymptomatic for cancer, and lacks the traditional laboratory risk factors for cancer.

A “normative” condition of a subject to whom a composition is to be administered means the condition of a subject before administration, even if the subject happens to be suffering from a disease.

“Ovarian cancer” is the malignant growth of abnormal cells/tissue that develops in a woman's ovary. Types of ovarian tumors include epithelial (including serous cell, mucinous, endometrioid, clear cell, undifferentiated, papillary serous, and Brenner cell) ovarian tumors, germ cell tumors (including teratomas (mature and immature), struma ovarii, carcinoid, dysgerminoma, embryonal cell carcinoma, endodermal sinus tumor, primary choriocarcinoma, and gonadoblastoma), and stromal tumors (including granulosa cell tumor, theca cell tumor, Sertoli-Leydig cell tumor, and hilar cell tumor). As defined herein, the term “ovarian cancer” includes Stage 1, Stage 2, Stage 3, and Stage 4 ovarian cancer as determined by the Tumor/Nodes/Metastases (“TNM”) system which takes into account the size of the tumor, the number of involved lymph nodes, and the presence of any other metastases, or the FIGO staging system which uses information obtained after surgery, which can include a total abdominal hysterectomy, removal of (usually) both ovaries and fallopian tubes, (usually) the omentum, and pelvic (peritoneal) washings for cytology.

A “panel” of genes is a set of genes including at least two constituents.

A “population of cells” refers to any group of cells wherein there is an underlying commonality or relationship between the members in the population of cells, including a group of cells taken from an organism or from a culture of cells or from a biopsy, for example.

“Positive predictive value” or “PPV” is calculated by TP/(TP+FP) or the true positive fraction of all positive test results. It is inherently impacted by the prevalence of the disease and pre-test probability of the population intended to be tested.

“Prostate cancer” is the malignant growth of abnormal cells in the prostate gland, capable of invading and destroying other prostate cells, and spreading (metastasizing) to other parts of the body, including bones and lymph nodes. As defined herein, the term “prostate cancer” includes Stage 1, Stage 2, Stage 3, and Stage 4 prostate cancer as determined by the Tumor/Nodes/Metastases (“TNM”) system which takes into account the size of the tumor, the number of involved lymph nodes, and the presence of any other metastases; or Stage A, Stage B, Stage C, and Stage D, as determined by the Jewitt-Whitmore system.

“Risk” in the context of the present invention, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of lower risk cohorts, across population divisions (such as tertiles, quartiles, quintiles, or deciles, etc.) or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, and/or the rate of occurrence of the event or conversion from one disease state to another, i.e., from a normal condition to cancer or from cancer remission to cancer, or from primary cancer occurrence to occurrence of a cancer metastasis. Risk evaluation can also comprise prediction of future clinical parameters, traditional laboratory risk factor values, or other indices of cancer results, either in absolute or relative terms in reference to a previously measured population. Such differing use may require different consituentes of a Gene Expression Panel (Precision Profile™) combinations and individualized panels, mathematical algorithms, and/or cut-off points, but be subject to the same aforementioned measurements of accuracy and performance for the respective intended use.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art. The sample is blood, urine, spinal fluid, lymph, mucosal secretions, prostatic fluid, semen, haemolymph or any other body fluid known in the art for a subject. The sample is also a tissue sample. The sample is or contains a circulating endothelial cell or a circulating tumor cell.

“Sensitivity” is calculated by TP/(TP+FN) or the true positive fraction of disease subjects.

“Skin cancer” is the growth of abnormal cells capable of invading and destroying other associated skin cells, and includes non-melanoma and melanoma.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less and statistically significant at a p-value of 0.10 or less. Such p-values depend significantly on the power of the study performed.

A “set” or “population” of samples or subjects refers to a defined or selected group of samples or subjects wherein there is an underlying commonality or relationship between the members included in the set or population of samples or subjects.

A “Signature Profile” is an experimentally verified subset of a Gene Expression Profile selected to discriminate a biological condition, agent or physiological mechanism of action.

A “Signature Panel” is a subset of a Gene Expression Panel (Precision Profile™), the constituents of which are selected to permit discrimination of a biological condition, agent or physiological mechanism of action.

A “subject” is a cell, tissue, or organism, human or non-human, whether in vivo, ex vivo or in vitro, under observation. As used herein, reference to evaluating the biological condition of a subject based on a sample from the subject, includes using blood or other tissue sample from a human subject to evaluate the human subject's condition; it also includes, for example, using a blood sample itself as the subject to evaluate, for example, the effect of therapy or an agent upon the sample.

A “stimulus” includes (i) a monitored physical interaction with a subject, for example ultraviolet A or B, or light therapy for seasonal affective disorder, or treatment of psoriasis with psoralen or treatment of cancer with embedded radioactive seeds, other radiation exposure, and (ii) any monitored physical, mental, emotional, or spiritual activity or inactivity of a subject.

“Therapy” includes all interventions whether biological, chemical, physical, metaphysical, or combination of the foregoing, intended to sustain or alter the monitored biological condition of a subject.

“TN” is true negative, which for a disease state test means classifying a non-disease or normal subject correctly.

“TP” is true positive, which for a disease state test means correctly classifying a disease subject.

The PCT patent application publication number WO 01/25473, published Apr. 12, 2001, entitled “Systems and Methods for Characterizing a Biological Condition or Agent Using Calibrated Gene Expression Profiles,” filed for an invention by inventors herein, and which is herein incorporated by reference, discloses the use of Gene Expression Panels (Precision Profiles™) for the evaluation of a biological condition (including with respect to health and disease).

In particular, the Gene Expression Panels (Precision Profiles™) described herein may be used, without limitation, for the determination of what particular cancer is present in an individual.

Advances in genomics, proteomics and molecular pathology have generated many candidate biomarkers with potential clinical value. Their use for cancer diagnosis could improve patient care. However, translation from bench to bedside outside of the research setting has proved more difficult than might have been expected. One obstacle has been the ability of the biomarkers to discriminate between different types and clinical stage of cancer. The present invention provides Gene Expression Panels (Precision Profiles™) for the evaluation or characterization of cancer and conditions related to cancer in a subject. In particular the Gene Expression Panels described herein provide for the discrimination between various cancers. Specifically the Gene Expression Panels (Precision Profiles™) described herein are capable of discrimination between the patient having skin cancer, lung cancer, colon cancer, prostate cancer, ovarian cancer, breast cancer, and cervical cancer.

### Skin Cancer

Skin cancer is the growth of abnormal cells capable of invading and destroying other associated skin cells. Skin cancer is the most common of all cancers, probably accounting for more than 50% of all cancers. Melanoma accounts for about 4% of skin cancer cases but causes a large majority of skin cancer deaths. The skin has three layers, the epidermis, dermis, and subcutis. The top layer is the epidermis. The two main types of skin cancer, non-melanoma carcinoma, and melanoma carcinoma, originate in the epidermis. Non-melanoma carcinomas are so named because they develop from skin cells other than melanocytes, usually basal cell carcinoma or a squamous cell carcinoma. Other types of non-melanoma skin cancers include Merkel cell carcinoma, dermatofibrosarcoma protuberans, Paget's disease, and cutaneous T-cell lymphoma. Melanomas develop from melanocytes, the skin cells responsible for making skin pigment called melanin. Melanoma carcinomas include superficial spreading melanoma, nodular melanoma, acral lentiginous melanoma, and lentigo maligna.

Basal cell carcinoma affects the skin's basal layer, the lowest layer of the epidermis. It is the most common type of skin cancer, accounting for more than 90 percent of all skin cancers in the United States. Basal cell carcinoma usually appears as a shiny translucent or pearly nodule, a sore that continuously heals and re-opens, or a waxy scar on the head, neck, arms, hands, and face. Occasionally, these nodules appear on the trunk of the body, usually as flat growths. Although this type of cancer rarely metastasizes, it can extend below the skin to the bone and cause considerable local damage. Squamous cell carcinoma is the second most common type of skin cancer. It is a malignant growth of the upper most layer of the epidermis and may appear as a crusted or scaly area of the skin with a red inflamed base that resembles a growing tumor, non-healing ulcer, or crusted-over patch of skin. It is typically found on the rim of the ear, face, lips, and mouth but can spread to other parts of the body. Squamous cell carcinoma is generally more aggressive than basal cell carcinoma, and requires early treatment to prevent metastasis. Although the cure rate for both basal cell and squamous cell carcinoma is high when properly treated, both types of skin cancer increase the risk for developing melanomas.

Melanoma is a more serious type of cancer than the more common basal cell or squamous cell carcinoma. Because most malignant melanoma cells still produce melanin, melanoma tumors are often shaded brown or black, but can also have no pigment. Melanomas often appear on the body as a new mole. Other symptoms of melanoma include a change in the size, shape, or color of an existing mole, the spread of pigmentation beyond the border of a mole or mark, oozing or bleeding from a mole, and a mole that feels itchy, hard, lumpy, swollen, or tender to the touch.

Melanoma is treatable when detected in its early stages. However, it metastasizes quickly through the lymph system or blood to internal organs. Once melanoma metastasizes, it becomes extremely difficult to treat and is often fatal. Although the incidence of melanoma is lower than basal or squamous cell carcinoma, it has the highest death rate and is responsible for approximately 75% of all deaths from skin cancer in general.

Cumulative sun exposure, i.e., the amount of time spent unprotected in the sun is recognized as the leading cause of all types of skin cancer. Additional risk factors include blond or red hair, blue eyes, fair complexion, many freckles, severe sunburns as a child, family history of melanoma, dysplastic nevi (i.e., multiple atypical moles), multiple ordinary moles (>50), immune suppression, age, gender (increased frequency in men), xeroderma pigmentosum (a rare inherited condition resulting in a defect from an enzyme that repairs damage to DNA), and past history of skin cancer.

Treatment of skin cancer varies according to type, location, extent, and aggressiveness of the cancer and can include any one or combination of the following procedures: surgical excision of the cancerous skin lesion to reduce the chance of recurrence and preserve healthy skin tissue; chemotherapy (e.g., dacarbazine, sorafnib), and radiation therapy. Additionally, even when widespread, melanoma can spontaneously regress. These rare instances seem to be related to a patient's developing immunity to the melanoma. Thus, much research in treatment of melanoma has focused on ways to get patients' mmune system to react to their cancer, e.g., immunotherapy (e.g., Interleukin-2 (IL-2) and Interferon (IFN)), autologous vaccine therapy, adoptive T-Cell therapy, and gene therapy (used alone or in combination with surgical procedures, chemotherapy, and/or radiation therapy).

Currently, the characterization of skin cancer, or conditions related to skin cancer is dependent on a person's ability to recognize the signs of skin cancer and perform regular self-examinations. An initial diagnosis is typically made from visual examination of the skin, a dermatoscopic exam, and patient feedback, and other questions about the patient's medical history. A definitive diagnosis of skin cancer and the stage of the disease's development can only be determined by a skin biopsy, i.e., removing a part of the lesion for microscopic examination of the cells, which causes the patient pain and discomfort. Metastatic melanomas can be detected by a variety of diagnostic procedures including X-rays, CT scans, MRIs, PET and PET/CTs, ultrasound, and LDH testing. However, once the cancer has metastasized, prognosis is very poor and can rapidly lead to death. Early detection of cancer, particularly melanoma, is crucial for a positive prognosis. Thus a need exists for better ways to diagnose and monitor the progression and treatment of skin cancer.

### Lung Cancer

Lung cancer is the leading cause of cancer deaths among both men and women. It is a fast growing and highly fatal disease. Nearly 60% of people diagnosed with lung cancer die within one year of diagnosis. Nearly 75% die within 2 years. There are two major types of lung cancer: small cell lung cancer (SCLC) and non-small cell lung cancer (NSCLC). If lung cancer has characteristics of both types it is called a mixed small/large cell carcinoma. Approximately 85% of lung cancers are NSCLC. There are 3 sub-types of NSCLC, which differ in size, shape, and biochemical make-up. Approximately 35-50% of all lung cancers are squamous cell carcinomas. This lung cancer is linked to smoking and is typically found near the bronchus. Adenocarcinomas (e.g., bronchioloalveolar carcinoma) account for approximately 40% of all lung cancers, and is usually found in the outer region of the lung. Large-cell undifferentiated carcinoma accounts for approximately 10-15% of all lung cancers. Large-cell undifferentiated carcinoma can appear in any part of the lung, and grows and spreads very quickly, resulting in poor prognosis.

SCLC accounts for approximately 15% of all lung cancers. SCLC often starts in the bronchi near the center of the chest and tends to spread widely through the body, quickly. The cancer cells can multiply quickly, form large tumors, and spread to lymph nodes and other organs such as the brain, adrenal glands, and liver. Thus, surgery is rarely an option, and is never used as the sole treatment modality.

In addition to the SCLC and NSCLC, other types of tumors can occur in the lungs. For example, carcinoid tumors of the lung account for fewer than 5% of lung tumors. Most are slow growing typical carcinoid tumors, which are generally cured by surgery. Cancers intermediate between the benign carcinoid tumors and SCLC are known as atypical carcinoid tumors. Other types of lung tumors include adenoid cystic carcinomas, hamartomas, lymphomas, sarcomas, and mesothelioma (tumor of the pleura (the layer of cells that line the outer surface of the lung)), which is associated with asbestos exposure.

The most important risk factor for lung cancer is smoking, including cigarette, cigar, pipe, marijuana, and hookah smoke. Despite popular belief, there is no evidence that smoking low tar or “light” cigarettes reduces the risk of lung cancer. Mentholated cigarettes may increase the risk of developing lung cancer. Additionally, non-smokers are at risk for lung cancer due to second hand smoke. Other risk factors include age (increased risk in the elderly population, nearly 70% of people diagnosed are over age 65); genetic predisposition; exposure to high levels of arsenic in drinking water, asbestos fibers, and/or long term radon contamination (each more pronounced in smokers); cancer causing agents in the workplace (e.g., radioactive ores, inhaled chemicals or minerals (e.g., arsenic, berrylium, vinyl chloride, nickel chromates, coal products, mustard gas, chloromethyl ethers, fuels such as gasoline, and diesel exhaust)); prior radiation therapy to the lungs; personal and family history of lung cancer; a diet low in fruits and vegetables (more pronounced in smokers); and air pollution.

Frequently, lung cancer remains asymptomatic until it reaches an advanced stage and spreads beyond the lungs. Once symptoms do start presenting, they include persistent cough; chest pain, often aggravated by deep breathing, coughing, or laughing; hoarseness; weight loss and loss of appetite; bloody or rust colored sputum; shortness of breath; recurring infections (e.g., bronchitis); new onset of wheezing; severe shoulder pain and/or Horner syndrome; and paraneoplastic syndromes (problems with distant organs due to hormone producing lung cancer). The most common paraneoplastic syndromes caused by NSCLC include hypercalcemia, causing urinary frequency, constipation, weakness, dizziness, confusion, and other CNS problems; hypertrophic osteoarthropathy (excess growth of certain bones); production of substances that activate the clotting cascade, leading to blood clots; and gynecomastia (excess breast growth in men). Additional symptoms may present when lung cancer spreads to distant organs causing symptoms such as bone pain, neurologicalchanges, jaundice, and masses near the surface of the body due to cancer spreading to the skin or lymph nodes.

SCLC and NSCLC are treated very differently. SCLC is mainly treated with chemotherapy, either alone or in combination with radiation. Surgery is rarely used in SCLC, and only when the cancer forms one localized tumor nodule with no spread to the lymph node or organs. For chemotherapy, cisplatin or carboplatin is usually combined with etoposide as the optimal treatment for SCLC, replacing older regimens of cyclophosphamide, doxorubicin, and to vincristine. Additionally, gemcitabine, paclitaxel, vinorelbine, topotecan, and irinotecan have shown promising results in some SCLC studies. After chemotherapy, radiation therapy can be used to kill small deposits of cancer that have not been eliminated. Radiation therapy (e.g., external beam radiation therapy, brachytherapy, and “gamma knife”), can also be used to relieve symptoms of lung cancer such as pain, bleeding, difficulty swallowing, cough, and problems caused by brain metastases.

In contrast with treatment for SCLC, surgery (lobectomy-removal of a lobe of the lung; pneumonectomy-removal of the entire lung; and segmentectomy resection-removing part of a lobe) is the only reliable method to cure NSCLC. Lymph nodes are also removed to assess the spread of cancer. More recently, a less invasive procedure called video assisted thoracic surgery has been used to remove early stage NSCLC.

In addition to surgery, chemotherapy is sometimes used to treat NSCLC. Cisplatin or carboplatin combined with gemcitabine, paclitaxel, docetaxel, etoposide, or vinorelbine has been effective in treating NSCLC. Recently, targeted therapy (drugs that interfere with the ability of the cancer cells to grow, e.g., gefitinib (Iressa™) and erlotinib (Tarceva™)) has shown some success in treating NSCLC in patients who are no longer responding to chemotherapy. Additionally, antiangionesis drugs (e.g., bevacizumab (Avastin™)) have recently been found to prolong survival of patients with advanced lung cancer when added to the standard chemotherapy regimen (however cannot be administered to patients with squamous cell cancer, because it leads to bleeding from this type of lung cancer).

Since individuals with lung cancer can be-asymptomatic while the disease progresses and metastasizes, screenings are essential to detect lung cancer at the earliest stage possible. Diagnosis for lung cancer is typically done through a combination of a medical history to check for risk factors and symptoms, physical exam to look for signs of lung cancer, imaging tests to look for tumors in the lungs or other organs, (e.g., chest X-ray, CT scan, MRI, PET, and bone scans), blood counts and blood chemistry, and invasive procedures that assist the physician to image the inside of the lungs and sample tissues/cells to determine whether a tumor is benign or malignant, and to determine the type of lung cancer (e.g., sputum cytology-microscopic examination of cells in coughed up phlegm; CT guided needle biopsy, bronchoscopy-viewing the inside of the bronchi through a flexible lighted tube; endobronchial ultrasound; endoscopic esophageal ultrasound; mediastinoscopy, mediastinotomy; thoracentesis; and thorascopy).

Because lung cancer spreads beyond the lungs before causing any symptoms, an effective screening program could save thousands of lives. To date, there is no lung cancer test that has been shown to prevent people from dying from this disease. Studies show that commonly used screening methods such as chest x-rays and sputum cytology are incapable of detecting lung cancer early enough to improve a person's chance for a cure. For this reason, lung cancer screening is not a routine practice for the general population, or even for people at increased risk, such as smokers. Even with the screening procedures currently available, it is nearly impossible to detect or verify a diagnosis of lung cancer in a non-invasive manner, and without causing the patient pain and discomfort. Thus, a need exists for better ways to diagnose and monitor the progression and treatment of lung cancer.

### Colorectal Cancer

Colorectal cancer is a type of cancer that develops in the gastrointestinal system (GI system), specifically in the colon, or the rectum. The GI system consists of the small intestine, the large intestine (also known as the colon), the rectum, and the anus. The colon is a muscular tube, about five feet long on average, and has four sections: the ascending colon which begins where the small bowel attaches to the colon and extends upward on the rights side of the abdomen; the transverse colon, which runs across the body from the right to left side in the upper abdomen; the descending colon, which continues downward on the left side; and the sigmoid colon, which joins the rectum, which in turn joins the anus. The wall of each of the sections of the colon and rectum has several layers of tissue. Colorectal cancer starts in the innermost layer of tissue of the colon or rectum and can grow through some or all of the other layers. The stage (i.e., the extent of spread) of colorectal cancer depends on how deeply it invades into these layers.

Colorectal cancer develops slowly over a period of several years, usually beginning as a non-cancerous or pre-cancerous polyp which develops on the lining of the colon or rectum. Certain kinds of polyps, called adenomatous polyps (or adenomas), are highly likely to become cancerous. Other kinds of polyps, called hyperplastic polyps and inflammatory polyps, indicate an increased chance of developing adenomatous polyps and cancer, particularly if growing in the ascending colon. A pre-cancerous condition known as dysplasia is common in people suffering from diseases which cause chronic inflammation in the colon, such as ulcerative colitis or Chrohn's Disease.

Over 95% of colorectal cancers are adenocarcinomas, a cancer of the glandular cells that line the inside layer of the wall of the colon and rectum. Other types of colorectal tumors include carcinoid tumors, which develop from hormone producing cells of the colon; gastrointestinal stromal tumors, which develop in the interstitial cells of Cajal within the wall of the colon; and lymphomas of the digestive system.

Once cancer forms within a colorectal polyp, it eventually grows into the wall of the colon or rectum. Once cancer cells are in the wall, they can grow into blood vessels or lymph vessels, at which point the cancer metastizes.

Colorectal cancer is the third most common cancer diagnosed in men and women, and is the second leading cause of cancer-related deaths in the United States. Risk factors for colorectal cancer include age (increased chance after age 50); personal history of colorectal cancer, polyps, or chronic inflammatory bowel disease; ethnic background (Jews of Eastern European descent have higher rates of colorectal cancer); a diet mostly from animal sources (high in fat); physical inactivity; obesity; smoking (30-40% increased risk for colorectal cancer); and high alcohol intake. Additionally, individuals with a family history of colorectal cancer have an increased risk for developing the disease. About 30% of people who develop colorectal cancer have disease that is familial. About another 10% of people who develop colorectal cancer have an inherited genetic susceptibility to the disease; approximately 3-5% of colorectal cancers are associated with a syndrome called hereditary non-polyposis colorectal cancer (HNPCC), approximately 1% of colorectal cancers are associated with an inherited syndrome called familial adenomatous polyposis (FAP).

FAP is a disease where people develop hundreds of polyps in their colon and rectum, typically between the ages of 5 and 40 years. Cancer develops in one or more of these polyps as early as age 20. By age 40, almost all people with FAP will have developed cancer if preventative surgery is not done. HNPCC also develops at a relatively young age. However, individuals with HNPCC develop only a few polyps. Women with HNPCC have a high risk of developing endometrial cancer. Other cancers associated with HNPCC include cancer of the ovary, stomach, small intestine, pancreas, kidney, ureter, and bile duct. The lifetime risk of developing colorectal cancer for people with HNPCC is about 80%, compared to near 100% for those with FAP.

From the time the first abnormal cells in polyps start to grow, it takes about 10-15 years for them to develop into colorectal cancer. An individual can live asymptomatic for several years with precancerous polyps that develop into colorectal cancer without knowing it. Once symptoms do start presenting, they include changes in bowel habits (e.g., constipation, diarrhea, narrowing of the stool), stomach cramping or bloating, bright red blood in stool, unexplained weight loss, constant fatigue, constant sensation of needing a bowel movement, nausea and vomiting, gaseousness, and anemia.

Treatment of colorectal cancer varies according to type, location, extent, and aggressiveness of the cancer, and can include any one or combination of the following procedures: surgery, radiation therapy, and chemotherapy, and targeted therapy (e.g., monoclonal antibodies). Surgery is the main treatment for colorectal cancer. At early stages it may be possible to remove cancerous polyps through a colonoscope, by passing a wire loop through the colonoscope to cut the polyp from the wall of the colon with an electrical current. The most common operation for colon cancer is a segmental resection, in which the cancer a length of the normal colon on either side of the cancer, and nearby lymph nodes are removed, and the remaining sections of the colon are reattached.

Radiation therapy uses high energy rays to destroy cancer cells, and is used after colorectal surgery to destroy small deposits of cancer that may not be detected during surgery, or when the cancer has attached to an internal organ or lining of the abdomen. Radiation therapy is also used to treat local recurrences of rectal cancer. Several types of radiation therapy are available, including external-beam radiation therapy, endocavitry radiation therapy, and brachytherapy. Radiation therapy is also often used after surgery in combination with chemotherapy.

Chemotherapy can also be used to shrink primary tumors, relieve symptoms of advanced colorectal cancer, or as an adjuvant therapy. Fluorouracil (5-FU) is the drug most often used to treat colon cancer. In adjuvant therapy, it is often administered with leucovorin via an IV injection regimen to increase its effectiveness. Capecitabine (Xeloda™) is an orally administered chemotherapeutic that is converted to 5-FU once it reaches the tumor site. Other chemotherapeutics which have been found to increase the effectiveness 5-FU and leucovorin when given in combination include Irinotecan (Camptosar™), and Oxaliplatin.

Targeted therapies such as monoclonal antibodies are being used more frequently to specifically attack cancer cells with fewer side effects than radiation therapy or chemotherapy. Monoclonal antibodies that have been approved for the treatment of colon cancer include Cetuximab (Erbitux™), and Bevacizumab (Avastin™).

Since individuals with colon cancer can live for several years asymptomatic while the disease progresses, regular screenings are essential to detect colorectal cancer at an early stage, or to prevent abnormal polyps from developing into colorectal cancer. Diagnosis for colorectal cancer is typically done through a combination of a medical history, physical exam, blood tests for anemia or tumor markers (e.g., carcinoembryonic antigen, or CA19-9); and one or more screening methods for polyps or abnormalities in the lining of the colorectal wall.

A number of different screening methods for colorectal cancer are available. However, most procedures are highly invasive and painful. Take home test kits such as the fecal occult blood test (FOBT), or fecal immunochemical test (FIT), use a chemical reaction to detect occult (hidden blood) in the feces due to ruptured blood vessels at the surface of colorectal polyps of adenomas or cancers, damaged by the passage of feces. However, since occult in the stool could be indicative of a variety of gastrointestinal disorders, a colonoscopy or sigmoidoscopy is necessary to verify that positive FOBT or FIT results are due to colorectal cancer.

A colonoscopy involves a colonoscope which is a longer version of a sigmoidoscope, connected to a camera or monitor, and is inserted through the rectum to enable a doctor to visualize the lining of the entire colon. Polyps detected by such screening methods can be removed through a colonoscope or biopsied to determine whether the polyp is cancerous, benign, or a result of inflammation.

Additional screening techniques include invasive imaging techniques such as a barium enema with air contrast, or virtual colonoscopy. A barium enema with air contrast involves pumping barium sulfate and air through the anus to partially fill and open up the colon, then x-ray to image the lining of the colon. Virtual colonoscopy uses only air pumped through the anus to distend the colon, then a helical or spiral CT scan to image the lining of the colon. Ultrasound, CT scan, PET scan, and MRI can also be used to image the lining of the colorectal wall. However, if abnormalities such as polyps are found by any such imaging technique, a procedure such as a colonoscopy or CT guided needle biopsy is still necessary to remove or biopsy the polyp. It is nearly impossible to detect or verify a diagnosis of colorectal cancer in a non-invasive manner, and without causing the patient pain and discomfort. Thus a need exists for better ways to diagnose and monitor the progression and treatment of colorectal cancer.

### Prostate Cancer

Prostate cancer is the most common cancer diagnosed among American men, with more than 234,000 new cases per year. As a man increases in age, his risk of developing prostate cancer increases exponentially. Under the age of 40, 1 in 1000 men will be diagnosed; between ages 40-59, 1 in 38 men will be diagnosed and between the ages of 60-69, 1 in 14 men will be diagnosed. More that 65% of all prostate cancers are diagnosed in men over 65 years of age. Beyond the significant human health concerns related to this dangerous and common form of cancer, its economic burden in the U.S. has been estimated at $8 billion dollars per year, with average annual costs per patient of approximately $12,000.

Prostate cancer is a heterogeneous disease, ranging from asymptomatic to a rapidly fatal metastatic malignancy. Survival of the patient with prostatic carcinoma is related to the extent of the tumor. When the cancer is confined to the prostate gland, median survival in excess of 5 years can be anticipated. Patients with locally advanced cancer are not usually curable, and a substantial fraction will eventually die of their tumor, though median survival may be as long as 5 years. If prostate cancer has spread to distant organs, current therapy will not cure it. Median survival is usually 1 to 3 years, and most such patients will die of prostate cancer. Even in this group of patients, however, indolent clinical courses lasting for many years may be observed. Other factors affecting the prognosis of patients with prostate cancer that may be useful in making therapeutic decisions include histologic grade of the tumor, patient's age, other medical illnesses, and PSA levels.

Early prostate cancer usually causes no symptoms. However, the symptoms that do present are often similar to those of diseases such as benign prostatic hypertrophy. Such symptoms include frequent urination, increased urination at night, difficulty starting and maintaining a steady stream of urine, blood in the urine, and painful urination. Prostate cancer may also cause problems with sexual function, such as difficulty achieving erection or painful ejaculation.

Currently, there is no single diagnostic test capable of differentiating clinically aggressive from clinically benign disease. Since individuals can have prostate cancer for several years and remain asymptomatic while the disease progresses and metastasizes, screenings are essential to detect prostate cancer at the earliest stage possible. Although early detection of prostate cancer is routinely achieved with physical examination and/or clinical tests such as serum prostate-specific antigen (PSA) test, this test is not definitive, since PSA levels can also be elevated due to prostate infection, enlargement, race and age effects. For example, a PSA level of 3 or less is considered in the normal range for a male under 60 years old, a level of 4 or less is considered normal for a male between the ages of 60-69, and a level of 5 or less is normal for males over the age of 70. Generally, the higher the level of PSA, the more likely prostate cancer is present. However, a PSA level above the normal range (depending on the age of the patient) could be due to benign prostatic disease. In such instances, a diagnosis would be impossible to confirm without biopsying the prostate and assigning a Gleason Score. Additionally, regular screening of asymptomatic men remains controversial since the PSA screening methods currently available are associated with high false-positive rates, resulting in unnecessary biopsies, which can result in significant morbidity.

Additionally, the clinical course of prostate cancer disease can be unpredictable, and the prognostic significance of the current diagnostic measures remains unclear. Furthermore, current tests do not reliably identify patients who are likely to respond to specific therapies—especially for cancer that has spread beyond the prostate gland. Thus, there is the need for tests which can aid in the diagnosis and monitor the progression and treatment of prostate cancer.

### Ovarian Cancer

Ovarian cancer is the fifth leading cause of cancer death in women, the leading cause of death from gynecological malignancy, and the second most commonly diagnosed gynecologic malignancy. Approximately 25,000 women in the United States are diagnosed with this disease each year.

Many types of tumors can start growing in the ovaries. Some are benign and never spread beyond the ovary while other types of ovarian tumors are malignant and can spread to other parts of the body. In general, ovarian tumors are named according to the kind of cells the tumor started from and whether the tumor is benign or cancerous. There are 3 main types of ovarian tumors: 1) germ cell tumors originate from the cells that produce the ova (eggs); 2) stromal tumors originate from connective tissue cells that hold the ovary together and produce the female hormones estrogen and progesterone; and 3) epithelial tumors originate from the cells that cover the outer surface of the ovary.

Cancerous epithelial tumors are called carcinomas. About 85% to 90% of ovarian cancers are epithelial ovarian carcinomas, and about 5% of ovarian cancers are germ cell tumors (including teratoma, dysgerminoma, endodermal sinus tumor, and choriocarcinoma). More than half of stromal tumors are found in women over age 50, but some occur in young girls. Types of malignant stromal tumors include granulosa cell tumors, granulosa-theca tumors, and Sertoli-Leydig cell tumors, which are usually considered low-grade cancers. Thecomas and fibromas are benign stromal tumors.

Ovarian cancer may spread by invading organs next to the ovaries such as the uterus or fallopian tubes), shedding (break off) from the main ovarian tumor and into the abdomen, or spreading through the lymphatic system to lymph nodes in the pelvis, abdomen, and chest, or through the bloodstream to organs such as the liver and lung. Cancerous cells which are shed into the naturally occurring fluid within the abdominal cavity have the potential to float in this fluid and frequently implant on other abdominal (peritoneal) structures including the uterus, urinary bladder, bowel, and lining of the bowel wall (omentum). These cells can begin forming new tumor growths before cancer is even suspected.

Early stage ovarian cancers are usually silent. However, when they do cause symptoms, these symptoms are typically non-specific, such as abdominal discomfort, abdominal swelling/bloating, increased gas, indigestion, lack of appetite, and/or nausea and vomiting. Symptoms presented during advanced stage ovarian cancer may include vaginal bleeding, weight gain/loss, abnormal menstrual cycles, back pain, and increased abdominal girth. Additional symptoms that may be associated with this disease include increased urinary frequency/urgency, excessive hair growth, fluid buildup in the lining around the lungs (Pleural effusions), and positive pregnancy readings in the absence of pregnancy (germ cell tumors only).

Because the symptoms of early stage ovarian cancer are non-specific, ovarian cancer in its early stages is often difficult to diagnose. Currently, there is no specific screening test for ovarian cancer. A blood test called CA-125 is sometimes useful in differential diagnosis of epithelial tumors or for monitoring the recurrence or progression of these tumors, but it has not been shown to be an effective method to screen for early-stage ovarian cancer and is currently not recommended for this use. Other tests for epithelial ovarian cancer that have been used include tumor markers BRCA-1/BRCA-2, Carcinoembrionic Antigen (CEA), galactosyltransferase, and Tissue Polypeptide Antigen (TPA).

More than 50% of women with ovarian cancer are diagnosed in the advanced stages of the disease because no cost-effective screening test for ovarian cancer exists. Additionally, ovarian cancer has a poor prognosis. It is disproportionately deadly because symptoms are vague and non-specific. The five-year survival rate for all stages is only 35% to 38%. A screening test capable of diagnosing ovarian cancer in early stages of the disease can increase five-year survival rates.

Furthermore, there is currently no test capable of reliably identifying patients who are likely to respond to specific therapies, especially for cancer that has spread beyond the ovarian gland. Thus, there is the need for tests which can aid in the diagnosis and monitor the progression and treatment of ovarian cancer.

### Breast Cancer

Breast cancer is cancer that forms in tissues of the breast, usually the ducts and lobules (glands that make milk). It occurs in both men and women, although male breast cancer is rare. Worldwide, it is the most common form of cancer in females, and is the second most fatal cancer in women, affecting, at some time in their lives, approximately one out of thirty-nine to one out of three women who reach age ninety in the Western world.

There are many different types of breast cancer, including ductal carcinoma, lobular carcinoma, inflammatory breast cancer, medullary carcinoma, colloid carcinoma, papillary carcinoma, and metaplastic carcinoma. Ductal carcinoma is a very common type of breast cancer in women. Ductal carcinoma refers to the development of cancer cells within the milk ducts of the breast. It comes in two forms: infiltrating ductal carcinoma (IDC), an invasive cell type; and ductal carcinoma in situ (DCIS), a noninvasive cancer. DCIS is the most common type of noninvasive breast cancer in women. IDC, formed in the ducts of breast in the earliest stage, is the most common, most heterogeneous invasive breast cancer cell type. It accounts for 80% of all types of breast cancer.

Early breast cancer can in some cases be painful. A lump under the arm or above the collarbone that does not go away may be present. Other possible symptoms include breast discharge, nipple inversion and changes in the skin overlying the breast. Breast cancer is often discovered before any symptoms are even present. Due to the high incidence of breast cancer among older women, screening is highly recommended and often routine in physical examinations of women, with mammograms for women over the age of 50. Current screening methods include breast self-examination, mammography ultrasound, and MRI.

Mammography is the modality of choice for screening of early breast cancer, and breast cancers detected by mammography are usually smaller than those detected clinically. While mammography has been shown to reduce breast cancer-related mortality by 20-30%, the test is not very accurate. Only a small fraction (5-10%) of abnormalities on mammograms turn out to be breast cancer. However, each suspicious mammogram requires a follow-up medical visit which typically includes a second mammogram, and other follow-up test procedures including sonograms, needle biopsies, or surgical biopsies. Most women who undergo these procedures find out that no breast cancer is present. Additionally, the number of unnecessary medical procedures involved in following up on a false positive mammography results creates an unnecessary economic burden.

Additionally, mammograms can give false negative results. A false negative result occurs when cancer is present and not diagnosed. Breast density and the experience, skill, and training of the doctor reading a mammogram are contributing factors which can lead to false negative results. Unless a patient were to receive a second opinion, a false negative mammography eventually results in advanced stage breast cancer which may be untreatable and/or fatal by the time it is detected. Thus, there is a need for tests which can aid in the diagnosis of breast cancer.

Furthermore, there is currently no test capable of reliably identifying patients who are likely to respond to specific therapies, especially for cancer that has spread beyond the breast tissue. Thus, there is also the need for tests which can aid in monitoring the progression and treatment of breast cancer.

### Cervical Cancer

Cervical cancer is a malignancy of the cervix. Most scientific studies have found that human papillomavirus (HPV) infection is responsible for virtually all cases of cervical cancer. Worldwide, cervical cancer is the third most common type of cancer in women. However, it is much less common in the United States because of routine use of Pap smears. There are two main types of cervical cancer: squamous cell cancer and adenocarcinoma, named after the type of cell that becomes cancerous. Squamous cells are the flat skin-like cells that cover the outer surface of the cervix (the ectocervix). Squamous cell cancer is the most common type of cervical cancer. Adenomatous cells are gland cells that produce mucus. The cervix has these gland cells scattered along the inside of the passageway that runs from the cervix to the womb. Adenocarinoma is a cancer of these gland cells.

Cervical cancer may present with abnormal vaginal bleeding or discharge. Other symptoms include weight loss, fatigue, pelvic pain, back pain, leg pain, single swollen leg, and bone fractures. However, symptoms may be absent until the cancer is in its advanced stages. Undetected, pre-cancerous changes can develop into cervical cancer and spread to the bladder, intestines, lungs, and liver. The development of cervical cancer is very slow. It starts as a pre-cancerous condition called dysplasia. This pre-cancerous condition can be detected by a Pap smear and is 100% treatable. While an effective screening tool, the Pap smear is an invasive procedure, and is incapable of offering a final diagnosis. Diagnosis of cervical cancer must be confirmed by surgically removing tissue from the cervix (colposcopy, or cone biopsy), which may also be a painful procedure, and one which causes the patient great discomfort. Thus, there is a need for non-invasive, pain-free tests which can aid in the diagnosis of cervical cancer.

Furthermore, there is currently no test capable of reliably identifying patients who are likely to respond to specific therapies, especially for advanced stage cervical cancer, or cancer that has spread beyond the cervical tissue. Thus, there is also the need for tests which can aid in monitoring the progression and treatment of cervical cancer.

Information on any condition of a particular patient and a patient's response to types and dosages of therapeutic or nutritional agents has become an important issue in clinical medicine today not only from the aspect of efficiency of medical practice for the health care industry but for improved outcomes and benefits for the patients. Thus, there is the need for tests which can aid in the diagnosis and monitor the progression and treatment of cancer, including but not limited to skin, lung, colon, prostate, ovarian, breast, and cervical cancer.

The Gene Expression Panels (Precision Profiles™) are referred to herein as the Precision Profile™ for Inflammatory Response, the Human Cancer General Precision Profile™, and the Precision Profile™ for EGR1. The Precision Profile™ for Inflammatory Response includes one or more genes, e.g., constituents, listed in Table A, whose expression is associated with inflammatory response and cancer. The Human Cancer General Precision Profile™ includes one or more genes, e.g., constituents, listed in Table B, whose expression is associated generally with human cancer (including without limitation prostate, breast, ovarian, cervical, lung, colon, and skin cancer). The Precision Profile™ for EGR1 includes one or more genes, e.g., constituents listed in Table C, whose expression is associated with the role early growth response (EGR) gene family plays in human cancer. The Precision Profile™ for EGR1 is composed of members of the early growth response (EGR) family of zinc finger transcriptional regulators; EGR1, 2, 3 & 4 and their binding proteins; NAB1 & NAB2 which function to repress transcription induced by some members of the EGR family of transactivators. In addition to the early growth response genes, The Precision Profile™ for EGR1 includes genes involved in the regulation of immediate early gene expression, genes that are themselves regulated by members of the immediate early gene family (and EGR1 in particular) and genes whose products interact with EGR1, serving as co-activators of transcriptional regulation.

It has been discovered that valuable and unexpected results may be achieved when the quantitative measurement of constituents is performed under repeatable conditions (within a degree of repeatability of measurement of better than twenty percent, preferably ten percent or better, more preferably five percent or better, and more preferably three percent or better). For the purposes of this description and the following claims, a degree of repeatability of measurement of better than twenty percent may be used as providing measurement conditions that are “substantially repeatable”. In particular, it is desirable that each time a measurement is obtained corresponding to the level of expression of a constituent in a particular sample, substantially the same measurement should result for substantially the same level of expression. In this manner, expression levels for a constituent in a Gene Expression Panel (Precision Profile™) may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar as defined herein. When both of these criteria are satisfied, then measurement of the expression level of one constituent may be meaningfully compared with measurement of the expression level of another constituent in a given sample and from sample to sample.

The evaluation or characterization of cancer is defined to be diagnosing or assessing the presence or absence of cancer,

Cancer and conditions related to cancer is evaluated by determining the level of expression (e.g., a quantitative measure) of an effective number (e.g., one or more) of constituents of a Gene Expression Panel (Precision Profile™) disclosed herein (i.e., Tables A-C). By an effective number is meant the number of constituents that need to be measured in order to discriminate between a subject having one type of cancer and the subject having another type of cancer. For example, the methods of the invention are capable of determining whether a subject has skin cancer or breast cancer. Preferably the constituents are selected as to discriminate (i.e., predict) between one type cancer and another type of cancer with at least 75% accuracy, more preferably 80%, 85%, 90%, 95%, 97%, 98%, 99% or greater accuracy.

The level of expression is determined by any means known in the art, such as for example quantitative PCR. The measurement is obtained under conditions that are substantially repeatable. Optionally, the qualitative measure of the constituent is compared to a reference or baseline level or value (e.g. a baseline profile set). In one embodiment, the reference or baseline level is a level of expression of one or more constituents in one or more subjects known to be suffering from breast, ovarian, cervical, prostate, lung, skin or colon cancer.

A reference or baseline level or value as used herein can be used interchangeably and is meant to be relative to a number or value derived from population studies, including without limitation, such subjects having similar age range, subjects in the same or similar ethnic group, sex, or, in female subjects, pre-menopausal or post-menopausal subjects, or relative to the starting sample of a subject undergoing treatment for a particular cancer. Such reference values can be derived from statistical analyses and/or risk prediction data of populations obtained from mathematical algorithms and computed indices of cancer. Reference indices can also be constructed and used using algorithms and other methods of statistical and structural classification.

In a further embodiment, such subjects are monitored and/or periodically retested for a diagnostically relevant period of time (“longitudinal studies”) following such test to verify continued presence of cancer. Such period of time may be one year, two years, two to five years, five years, five to ten years, ten years, or ten or more years from the initial testing date for determination of the reference or baseline value. Furthermore, retrospective measurement of cancer associated genes in properly banked historical subject samples may be used in establishing these reference or baseline values, thus shortening the study time required, presuming the subjects have been appropriately followed during the intervening period through the intended horizon of the product claim.

In another embodiment, the reference or baseline value is an index value or a baseline value. An index value or baseline value is a composite sample of an effective amount of cancer associated genes from one or more subjects who have a particular type of cancer.

A Gene Expression Panel (Precision Profile™) is selected in a manner so that quantitative measurement of RNA or protein constituents in the Panel constitutes a measurement of a biological condition of a subject. In one kind of arrangement, a calibrated profile data set is employed. Each member of the calibrated profile data set is a function of (i) a measure of a distinct constituent of a Gene Expression Panel (Precision Profile™) and (ii) a baseline quantity.

Additional embodiments relate to the use of an index or algorithm resulting from quantitative measurement of constituents, and optionally in addition, derived from either expert analysis or computational biology (a) in the analysis of complex data sets; (b) to control or normalize the influence of uninformative or otherwise minor variances in gene expression values between samples or subjects; (c) to simplify the characterization of a complex data set for comparison to other complex data sets, databases or indices or algorithms derived from complex data sets; and (d) to monitor a biological condition of a subject.

### The Subject

The methods disclosed herein may be applied to cells of humans, mammals or other organisms without the need for undue experimentation by one of ordinary skill in the art because all cells transcribe RNA and it is known in the art how to extract RNA from all types of cells.

A subject can include those who have not been previously diagnosed as having skin, lung, colon, prostate, ovarian, breast, or cervical cancer. Alternatively, a subject can also include those who have already been diagnosed as having skin, lung, colon, prostate, ovarian, breast, or cervical cancer.

Diagnosis of skin cancer is made, for example, from any one or combination of the following procedures: a medical history; a visual examination of the skin looking for common features of cancerous skin lesions, including but not limited to bumps, shiny translucent, pearly, or red nodules, a sore that continuously heals and re-opens, a crusted or scaly area of the skin with a red inflamed base that resembles a growing tumor, a non-healing ulcer, crusted-over patch of skin, new moles, changes in the size, shape, or color of an existing mole, the spread of pigmentation beyond the border of a mole or mark, oozing or bleeding from a mole, and a mole that feels itchy, hard, lumpy, swollen, or tender to the touch; a dermatoscopic exam; imaging techniques including X-rays, CT scans, MRIs, PET and PET/CTs, ultrasound, and LDH testing; and biopsy, including shave, punch, incisional, and excsisional biopsy.

Diagnosis of lung cancer is made, for example, from any one or combination of the following procedures: a medical history, physical exam, blood counts and blood chemistry, and screening and tissue sampling procedures such as sputum cytology, CT guided needle biopsy, bronchoscopy, endobronchial ultrasound, endoscopic esophageal ultrasound, mediastinoscopy, mediastinotomy, thoracentesis, and thorascopy.

Diagnosis of colorectal cancer is made, for example, from any one or combination of the following procedures: a medical history; physical exam; blood tests for anemia or tumor markers (e.g., carcinoembryonic antigen, or CA19-9); and one or more screening methods for polyps or abnormalities in the lining of the colorectal wall. Screening methods for polyps or abnormalities include but are not limited to: digital rectal examination (DRE); fecal occult blood test (FOBT); fecal immunochemical test (FIT); colonoscopy or sigmoidoscopy; barium enema with air contrast; virtual colonoscopy; biopsy (e.g., CT guided needle biopsy); and imaging techniques (e.g., ultrasound, CT scan, PET scan, and MRI).

Diagnosis of prostate cancer is made, for example, from any one or combination of the following procedures: a medical history, physical examination, e.g., digital rectal examination, blood tests, e.g., a PSA test, and screening tests and tissue sampling procedures e.g., cytoscopy and transrectal ultrasonography, and biopsy, in conjunction with Gleason Score.

Diagnosis of ovarian cancer is made, for example, from any one or combination of the following procedures: a medical history, physical examination, an abdominal and/or pelvic exam, blood tests (e.g., CA-125 levels), ultrasound, and biopsy.

Diagnosis of breast cancer is made, for example, from any one or combination of the following procedures: a medical history, physical examination, breast examination, mammography, chest x-ray, bone scan, CT, MRI, PET scanning, blood tests (e.g., CA-15.3 levels (carbohydrate antigen 15.3, and epithelial mucin)) and biopsy (including fine-needle aspiration, nipples aspirates, ductal lavage, core needle biopsy, and local surgical biopsy).

Diagnosis of cervical cancer is made, for example, from any one or combination of the following procedures: a medical history, a Pap smear, and biopsy procedures (including cone biopsy and colposcopy).

A subject can also include those who are suffering from, or at risk of developing skin cancer or a condition related to skin cancer (e.g., melanoma), such as those who exhibit known risk factors skin cancer. Known risk factors for skin cancer include, but are not limited to cumulative sun exposure, blond or red hair, blue eyes, fair complexion, many freckles, severe sunburns as a child, family history of skin cancer (e.g., melanoma), dysplastic nevi, atypical moles, multiple ordinary moles (>50), immune suppression, age, gender (increased frequency in men), xeroderma pigmentosum (a rare inherited condition resulting in a defect from an enzyme that repairs damage to DNA), and past history of skin cancer.

A subject can also include those who are suffering from different stages of skin cancer, e.g., Stage 1 through Stage 4 melanoma. An individual diagnosed with Stage 1 indicates that no lymph nodes or lymph ducts contain cancer cells (i.e., there are no positive lymph nodes) and there is no sign of cancer spread. In this stage, the primary melanoma is less than 2.0 mm thick or less than 1.0 mm thick and ulcerated, i.e., the covering layer of the skin over the tumor is broken. Stage 2 melanomas also have no sign of spread or positive lymph node status. Stage 2 melanomas are over 2.0 mm thick or over 1.0 mm thick and ulcerated. Stage 3 indicates all melanomas where there are positive lymph nodes, but no sign of the cancer having spread anywhere else in the body. Stage 4 melanomas have spread elsewhere in the body, away from the primary site.

Optionally, the subject has been previously treated with a surgical procedure for removing skin cancer or a condition related to skin cancer (e.g., melanoma), including but not limited to any one or combination of the following treatments: cryosurgery, i.e., the process of freezing with liquid nitrogen; curettage and electrodessication, i.e., the scraping of the lesion and destruction of any remaining malignant cells with an electric current; removal of a lesion layer-by-layer down to normal margins (Moh's surgery). Optionally, the subject has previously been treated with any one or combination of the following therapeutic treatments: chemotherapy (e.g., dacarbazine, sorafnib); radiation therapy; immunotherapy (e.g., Interleukin-2 and/or Interfereon to boost the body's immune reaction to cancer cells); autologous vaccine therapy (where the patient's own tumor cells are made into a vaccine that will cause the patient's body to make antibodies against skin cancer); adoptive T-cell therapy (where the patient's T-cells that target melanocytes are extracted then expanded to large quantities, then infused back into the patient); and gene therapy (modifying the genetics of tumors to make them more susceptible to attacks by cancer-fighting drugs); or any of the agents previously described; alone, or in combination with a surgical procedure for removing skin cancer, as previously described.

A subject can also include those who are suffering from, or at risk of developing lung cancer or a condition related to lung cancer, such as those who exhibit known risk factors for lung cancer or conditions related to lung cancer. Known risk factors for lung cancer include, but are not limited to: smoking, including cigarette, cigar, pipe, marijuana, and hookah smoke; second hand smoke; age (increased risk in the elderly population over age 65); genetic predisposition; exposure to high levels of arsenic in drinking water, asbestos fibers, and/or long term radon contamination (each more pronounced in smokers); cancer causing agents in the workplace (e.g., radioactive ores, inhaled chemicals or minerals (e.g., arsenic, berrylium, vinyl chloride, nickel chromates, coal products, mustard gas, chloromethyl ethers, fuels such as gasoline, and diesel exhaust)); prior radiation therapy to the lungs; personal and family history of lung cancer; diet low in fruits and vegetables (more pronounced in smokers); and air pollution.

Optionally, the subject has been previously treated with a surgical procedure for removing lung cancer or a condition related to lung cancer, including but not limited to any one or combination of the following treatments: lobectomy (removal of a lobe of the lung), pneumonectomy (removal of the entire lung), segmentectomy resection (removing part of a lobe), video assisted thoracic surgery, craniotomy, and pleurodesis. Optionally, the subject has previously been treated with any one or combination of the following therapeutic treatments: radiation therapy (e.g., external beam radiation therapy, brachytherapy and “gamma knife”), alone, in combination, or in succession with chemotherapy (e.g., cisplatin or carboplatin is combined with etoposide; cisplatin or carboplatin combined with gemcitabine, paclitaxel, docetaxel, etoposide, or vinorelbine; cyclophosphamide, doxorubicin, vincristine, gemcitabine, paclitaxel, vinorelbine, topotecan, irinotecan), alone, in combination or in succession with targeted therapy (e.g., gefitinib (Iressa™), erlotinib (Tarceva™) and bevacizumab (Avastin™) Optionally, radiation therapy, chemotherapy, and/or targeted therapy may be alone, in combination, or in succession with a surgical procedure for removing lung cancer. Optionally, the subject may be treated with any of the agents previously described; alone, or in combination with a surgical procedure for removing lung cancer and/or radiation therapy as previously described.

A subject can also include those who are suffering from, or at risk of developing colorectal cancer or a condition related to colorectal cancer, such as those who exhibit known risk factors for colorectal cancer or conditions related to colorectal cancer. Known risk factors for colorectal cancer include, but are not limited to: age (increased chance after age 50); personal history of colorectal cancer, polyps, or chronic inflammatory bowel disease; ethnic background (Jews of Eastern European descent have higher rates of colorectal cancer); a diet mostly from animal sources (high in fat); physical inactivity; obesity; smoking (30-40% increased risk for colorectal cancer); high alcohol intake; and family history of colorectal cancer, hereditary polyposis colorectal cancer, or familial adenomatous polyposis.

Optionally, the subject has been previously treated with a surgical procedure for removing colorectal cancer or a condition related to colorectal cancer, including but not limited to any one or combination of the following treatments: laparoscopic surgery, colonic segmental resection, polypectomy and local excision to remove superificial cancer and polyps, local transanal resection, lower anterior or abdominoperineal resection, colo-anal anastomosis, coloplasty, abdominoperineal resection, pelvic exteneration, and urostomy. Optionally, the subject has previously been treated with a therapeutic agent such as radiation therapy (e.g., external beam radiation therapy, endocavitary radiation therapy, and brachytherapy), chemotherapy (e.g., 5-FU, Leucovorin, Capecitabine (Xeloda™), Irinotecan (Camptosar™), and/or Oxaliplatin (Eloxitan™)), and targeted therapies (e.g., Cetuximab (Erbitux™), or Bevacizumab (Avastin™)), alone, in combination, or in succession with a surgical procedure for removing colorectal cancer. Optionally, the subject may be treated with any of the agents previously described; alone, or in combination with a surgical procedure for removing colorectal cancer and/or radiation therapy as previously described.

A subject can also include those who are suffering from, or at risk of developing prostate cancer or a condition related to prostate cancer, such as those who exhibit known risk factors for prostate cancer or conditions related to prostate cancer. Known risk factors for prostate cancer include, but are not limited to: age (increased risk above age 50), race (higher prevalence among African American men), nationality (higher prevalence in North America and northwestern Europe), family history, and diet (increased risk with a high animal fat diet).

Optionally, the subject has been previously treated with a surgical procedure for removing prostate cancer or a condition related to prostate cancer, including but not limited to any one or combination of the following treatments: prostatectomy (including radical retropubic and radical perineal prostatectomy), transurethral resection, orchiectomy, and cryosurgery. Optionally, the subject has previously been treated with radiation therapy including but not limited to external beam radiation therapy and brachytherapy). Optionally, the subject has been treated with hormonal therapy, including but not limited to orchiectomy, anti-androgen therapy (e.g., flutamide, bicalutamide, nilutamide, cyproterone acetate, ketoconazole and aminoglutethimide), and GnRH agonists (e.g., leuprolide, goserelin, triptorelin, and buserelin). Optionally, the subject has previously been treated with chemotherapy for palliative care (e.g., docetaxel with a corticosteroid such as prednisone). Optionally, the subject has previously been treated with any one or combination of such radiation therapy, hormonal therapy, and chemotherapy, as previously described, alone, in combination, or in succession with a surgical procedure for removing prostate cancer as previously described. Optionally, the subject may be treated with any of the agents previously described; alone, or in combination with a surgical procedure for removing prostate cancer and/or radiation therapy as previously described.

A subject can also include those who are suffering from, or at risk of developing ovarian cancer or a condition related to ovarian cancer, such as those who exhibit known risk factors for ovarian cancer or conditions related to ovarian cancer. Known risk factors for ovarian cancer include, but are not limited to: age (increased risk above age 55), family history of ovarian cancer, personal history of breast, uterus, colon, or rectal cancer, menopausal hormone therapy, and women who have never been pregnant.

Optionally, the subject has been previously treated with a surgical procedure for removing ovarian cancer or a condition related to ovarian cancer, including but not limited to any one or combination of the following treatments: unilateral oophorectomy, bilateral oophorectomy, salpingectomy, hysterectomy, unilateral salpingo-oophorectomy, and debulking surgery. Optionally, the subject has previously been treated with chemotherapy, including but not limited to a platinum derivative with a taxane, alone or in combination with a surgical procedure, as previously described, Optionally, the subject may be treated with any of the agents previously described; alone, or in combination with a surgical procedure for removing ovarian cancer, as previously described.

A subject can also include those who are suffering from, or at risk of developing breast cancer or a condition related to breast cancer, such as those who exhibit known risk factors for breast cancer or conditions related to breast cancer. Known risk factors for breast cancer include, but are not limited to: gender (higher susceptibility women than in men), age (increased risk with age, especially age 50 and over), inherited genetic predisposition (mutations in the BRCA1 and BRCA2 genes), alcohol consumption, and exposure to environmental factors (e.g., chemicals used in pesticides, cosmetics, and cleaning products).

Optionally, the subject has been previously treated with a surgical procedure for removing breast cancer or a condition related to breast cancer, including but not limited to any one or combination of the following treatments: a lumpectomy, mastectomy, and removal of the lymph nodes in the axilla. Optionally, the subject has previously been treated with chemotherapy (including but not limited to tamoxifen and aromatase inhibitors) and/or radiation therapy (e.g., gamma ray and brachytherapy), alone, in combination with, or in succession to a surgical procedure, as previously described. Optionally, the subject may be treated with any of the agents previously described; alone, or in combination with a surgical procedure for removing breast cancer, as previously described.

Optionally, the subject has been previously treated with a surgical procedure for removing cervical cancer or a condition related to cervical cancer, including but not limited to any one or combination of the following treatments: LEEP (Loop Electrosurgical Excision Procedure), cryotherapy—freezes abnormal cells, and laser therapy.

A subject can also include those who are suffering from, or at risk of developing cervical cancer or a condition related to cervical cancer, such as those who exhibit known risk factors for cervical cancer or conditions related to cervical cancer. Known risk factors for cervical cancer include but are not limited to: human papillomavirus infection, smoking, HIV infection, chlamydia infection, dietary factors, oral contraceptives, multiple pregnancies, use of the hormonal drug diethylstilbestrol (DES) and a family history of cervical cancer.

Optionally, the subject has previously been treated with chemotherapy (including but not limited to 5-FU, Cisplatin, Carboplatin, Ifosfamide, Paclitaxel, and Cyclophosphamide) and/or radiation therapy (internal and/or external), alone, in combination with, or in succession to a surgical procedure, as previously described. Optionally, the subject may be treated with any of the agents previously described; alone, or in combination with a surgical procedure for removing cervical cancer, as previously described.

### Selecting Constituents of a Gene Expression Panel (Precision Profile™)

The general approach to selecting constituents of a Gene Expression Panel (Precision Profile™) has been described in PCT application publication number WO 01/25473, incorporated herein in its entirety. A wide range of Gene Expression Panels (Precision Profiles™) have been designed and experimentally validated, each panel providing a quantitative measure of biological condition that is derived from a sample of blood or other tissue. For each panel, experiments have verified that a Gene Expression Profile using the panel's constituents is informative of a biological condition. (It has also been demonstrated that in being informative of biological condition, the Gene Expression Profile is used, among other things, to measure the effectiveness of therapy, as well as to provide a target for therapeutic intervention).

In addition to the Precision Profile™ for the Precision Profile™ for Inflammatory Response (Table A), the Human Cancer General Precision Profile™ (Table B), and the Precision Profile™ for EGR1 (Table C), a include relevant genes which may be selected for a given Precision Profiles™, such as the Precision Profiles™ demonstrated herein to be useful in the evaluation of breast, ovarian, cervical, prostate, lung, skin or colon cancer cancer.

### Inflammation and Cancer

Evidence has shown that cancer in adults arises frequently in the setting of chronic inflammation. Epidemiological and experimental studies provide strong support for the concept that inflammation facilitates malignant growth. Inflammatory components have been shown to 1) induce DNA damage, which contributes to genetic instability (e.g., cell mutation) and transformed cell proliferation (Balkwill and Mantovani, Lancet 357:539-545 (2001)); 2) promote angiogenesis, thereby enhancing tumor growth and invasiveness (Coussens L. M. and Z. Werb, Nature 429:860-867 (2002)); and 3) impair myelopoiesis and hemopoiesis, which cause immune dysfunction and inhibit immune surveillance (Kusmartsev and Gabrilovic, Cancer Immunol. Immunother. 51:293-298 (2002); Serafini et al., Cancer Immunol. Immunther. 53:64-72 (2004)).

Studies suggest that inflammation promotes malignancy via proinflammatory cytokines, including but not limited to IL-113, which enhance immune suppression through the induction of myeloid suppressor cells, and that these cells down regulate immune surveillance and allow the outgrowth and proliferation of malignant cells by inhibiting the activation and/or function of tumor-specific lymphocytes. (Bunt et al., J. Immunol. 176: 284-290 (2006). Such studies are consistent with findings that myeloid suppressor cells are found in many cancer patients, including lung and breast cancer, and that chronic inflammation in some of these malignancies may enhance malignant growth (Coussens L. M. and Z. Werb, 2002).

Additionally, many cancers express an extensive repertoire of chemokines and chemokine receptors, and may be characterized by dis-regulated production of chemokines and abnormal chemokine receptor signaling and expression. Tumor-associated chemokines are thought to play several roles in the biology of primary and metastatic cancer such as: control of leukocyte infiltration into the tumor, manipulation of the tumor immune response, regulation of angiogenesis, autocrine or paracrine growth and survival factors, and control of the movement of the cancer cells. Thus, these activities likely contribute to growth within/outside the tumor microenvironment and to stimulate anti-tumor host responses.

As tumors progress, it is common to observe immune deficits not only within cells in the tumor microenvironment but also frequently in the systemic circulation. Whole blood contains representative populations of all the mature cells of the immune system as well as secretory proteins associated with cellular communications. The earliest observable changes of cellular immune activity are altered levels of gene expression within the various immune cell types. Immune responses are now understood to be a rich, highly complex tapestry of cell-cell signaling events driven by associated pathways and cascades—all involving modified activities of gene transcription. This highly interrelated system of cell response is immediately activated upon any immune challenge, including the events surrounding host response to breast, ovarian, cervical, prostate, lung, skin or colon cancer cancer and treatment. Modified gene expression precedes the release of cytokines and other immunologically important signaling elements.

As such, inflammation genes, such as the genes listed in the Precision Profile™ for Inflammatory Response (Table A) are useful for distinguishing between one type cancer and another type of cancer, in addition to the other gene panels, i.e., Precision Profiles™, described herein.

### Early Growth Response Gene Family and Cancer

The early growth response (EGR) genes are rapidly induced following mitogenic stimulation in diverse cell types, including fibroblasts, epithelial cells and B lymphocytes. The EGR genes are members of the broader “Immediate Early Gene” (IEG) family, whose genes are activated in the first round of response to extracellular signals such as growth factors and neurotransmitters, prior to new protein synthesis. The IEG's are well known as early regulators of cell growth and differentiation signals, in addition to playing a role in other cellular processes. Some other well characterized members of the IEG family include the c-myc, c-fos and c-jun oncogenes. Many of the immediate early gene products function as transcription factors and DNA-binding proteins, though other IEG's also include secreted proteins, cytoskeletal proteins and receptor subunits. EGR1 expression is induced by a wide variety of stimuli. It is rapidly induced by mitogens such as platelet derived growth factor (PDGF), fibroblast growth factor (FGF), and epidermal growth factor (EGF), as well as by modified lipoproteins, shear/mechanical stresses, and free radicals. Interestingly, expression of the EGR1 gene is also regulated by the oncogenes v-raf, v-fps and v-src as demonstrated in transfection analysis of cells using promoter-reporter constructs. This regulation is mediated by the serum response elements (SREs) present within the EGR1 promoter region. It has also been demonstrated that hypoxia, which occurs during development of cancers, induces EGR1 expression. EGR1 subsequently enhances the expression of endogenous EGFR, which plays an important role in cell growth (over-expression of EGFR can lead to transformation). Finally, EGR1 has also been shown to be induced by Smad3, a signaling component of the TGFB pathway.

In its role as a transcriptional regulator, the EGR1 protein binds specifically to the G+C rich EGR consensus sequence present within the promoter region of genes activated by EGR1. EGR1 also interacts with additional proteins (CREBBP/EP300) which co-regulate transcription of EGR1 activated genes. Many of the genes activated by EGR1 also stimulate the expression of EGR1, creating a positive feedback loop. Genes regulated by EGR1 include the mitogens: platelet derived growth factor (PDGFA), fibroblast growth factor (FGF), and epidermal growth factor (EGF) in addition to TNF, IL2, PLAU, ICAM1, TP53, ALOX5, PTEN, FN1 and TGFB1.

As such, early growth response genes, or genes associated therewith, such as the genes listed in the Precision Profile™ for EGR1 (Table C) are useful for distinguishing between one type of cancer and another type of, in addition to the other gene panels, i.e., Precision Profiles™, described herein.

In general, panels may be constructed and experimentally validated by one of ordinary skill in the art in accordance with the principles articulated in the present application.

### Gene Expression Profiles Based on Gene Expression Panels of the Present Invention

Tables A1a-A18a were derived from a study of the gene expression patterns based on the Precision Profile™ for Inflammatory Response (Table A), and Tables and B1a-B18a were derived from a study of the gene expression patterns based on the Human Cancer General Precision Profile™ (Table B), for the following 18 combinations of cancer versus cancer comparisons (described in Examples 3 and 4, respectively, below): breast cancer vs. melanoma; breast cancer vs. ovarian cancer; cervical cancer vs. breast cancer; cervical cancer vs. colon cancer; cervical cancer vs. melanoma; cervical cancer vs. ovarian cancer; colon cancer vs. melanoma; lung cancer vs. breast cancer; lung cancer vs. cervical cancer; lung cancer vs. colon cancer; lung cancer vs. melanoma; lung cancer vs. ovarian cancer; lung cancer vs. prostate cancer; ovarian cancer vs. colon cancer; ovarian cancer vs. melanoma; prostate cancer vs. colon cancer; prostate cancer vs. melanoma; and breast cancer vs. colon cancer.

Table A1a lists all 1 and 2-gene models capable of distinguishing between subjects with breast cancer and melanoma (active disease, all stages) with at least 75% accuracy. Table Ata lists all 1 and 2-gene models capable of distinguishing between subjects with breast cancer and ovarian cancer with at least 75% accuracy. Table A3a lists all 1 and 2-gene models capable of distinguishing between subjects with cervical cancer and breast cancer with at least 75% accuracy. Table A4a lists all 1 and 2-gene models capable of distinguishing between subjects with cervical cancer and colon cancer with at least 75% accuracy. Table A5a lists all 1 and 2-gene models capable of distinguishing between subjects with cervical cancer and melanoma (active disease, all stages) with at least 75% accuracy. Table A6a lists all 1 and 2-gene models capable of distinguishing between subjects with cervical cancer and ovarian cancer with at least 75% accuracy. Table A1a lists all 1 and 2-gene models capable of distinguishing between subjects with colon cancer and melanoma (active disease, all stages) with at least 75% accuracy. Table A8a lists all 1 and 2-gene models capable of distinguishing between subjects with lung cancer and breast cancer with at least 75% accuracy. Table A9a lists all 1 and 2-gene models capable of distinguishing between subjects with lung cancer and cervical cancer with at least 75% accuracy. Table A10a lists all 1 and 2-gene models capable of distinguishing between subjects with lung cancer and colon cancer with at least 75% accuracy. Table A11a lists all 1 and 2-gene models capable of distinguishing between subjects with lung cancer and melanoma (active disease, all stages) with at least 75% accuracy. Table A12a lists all 1 and 2-gene models capable of distinguishing between subjects with lung cancer and ovarian cancer with at least 75% accuracy. Table A13a lists all 1 and 2-gene models capable of distinguishing between subjects with lung cancer and prostate cancer with at least 75% accuracy. Table A14a lists all 1 and 2-gene models capable of distinguishing between subjects with ovarian cancer and colon cancer with at least 75% accuracy. Table A15a lists all 1 and 2-gene models capable of distinguishing between subjects with ovarian cancer and melanoma (active disease, all stages) with at least 75% accuracy. Table A16a lists all 1 and 2-gene models capable of distinguishing between subjects with prostate cancer and colon cancer with at least 75% accuracy. Table All a lists all 1 and 2-gene models capable of distinguishing between subjects with prostate cancer and melanoma (active disease, all stages) with at least 75% accuracy. Table A18a lists all 1 and 2-gene models capable of distinguishing between subjects with breast cancer and colon cancer with at least 75% accuracy.

Table B1a lists all 1 and 2-gene models capable of distinguishing between subjects with breast cancer and melanoma (active disease, stages 2-4) with at least 75% accuracy. Table B2a lists all 1 and 2-gene models capable of distinguishing between subjects with breast cancer and ovarian cancer with at least 75% accuracy. Table B3a lists all 1 and 2-gene models capable of distinguishing between subjects with cervical cancer and breast cancer with at least 75% accuracy. Table B4a lists all 1 and 2-gene models capable of distinguishing between subjects with cervical cancer and colon cancer with at least 75% accuracy. Table B5a lists all 1 and 2-gene models capable of distinguishing between subjects with cervical cancer and melanoma (active disease, stages 2-4) with at least 75% accuracy. Table B6a lists all 1 and 2-gene models capable of distinguishing between subjects with cervical cancer and ovarian cancer with at least 75% accuracy. Table B7a lists all 1 and 2-gene models capable of distinguishing between subjects with colon cancer and melanoma (active disease, stages 2-4) with at least 75% accuracy. Table B8a lists all 1 and 2-gene models capable of distinguishing between subjects with lung cancer and breast cancer with at least 75% accuracy. Table B9a lists all 1 and 2-gene models capable of distinguishing between subjects with lung cancer and cervical cancer with at least 75% accuracy. Table B10a lists all 1 and 2-gene models capable of distinguishing between subjects with lung cancer and colon cancer with at least 75% accuracy. Table B11a lists all 1 and 2-gene models capable of distinguishing between subjects with lung cancer and melanoma (active disease, stages 2-4) with at least 75% accuracy. Table B12a lists all 2-gene models capable of distinguishing between subjects with lung cancer and ovarian cancer with at least 75% accuracy. Table B13a lists all 1 and 2-gene models capable of distinguishing between subjects with lung cancer and prostate cancer with at least 75% accuracy. Table B14a lists all 1 and 2-gene models capable of distinguishing between subjects with ovarian cancer and colon cancer with at least 75% accuracy. Table B15a lists all 1 and 2-gene models capable of distinguishing between subjects with ovarian cancer and melanoma (active disease, stages 2-4) with at least 75% accuracy. Table B16a lists all 1 and 2-gene models capable of distinguishing between subjects with prostate cancer and colon cancer with at least 75% accuracy. Table B17a lists all 1 and 2-gene models capable of distinguishing between subjects with prostate cancer and melanoma (active disease, stages 2-4) with at least 75% accuracy. Table B18a lists all 2-gene models capable of distinguishing between subjects with breast cancer and colon cancer with at least 75% accuracy.

Tables C1a-C17a were derived from a study of the gene expression patterns based on the Precision Profile™ for EGR1 (Table C) for the following 17 combinations of cancer versus cancer comparisons, described in Example 5 below: breast cancer vs. melanoma; breast cancer vs. ovarian cancer; cervical cancer vs. breast cancer; cervical cancer vs. colon cancer; cervical cancer vs. melanoma; cervical cancer vs. ovarian cancer; colon cancer vs. melanoma; lung cancer vs. breast cancer; lung cancer vs. cervical cancer; lung cancer vs. colon cancer; lung cancer vs. melanoma; lung cancer vs. ovarian cancer; lung cancer vs. prostate cancer; ovarian cancer vs. colon cancer; ovarian cancer vs. melanoma; prostate cancer vs. colon cancer; and prostate cancer vs. melanoma.

Table C1a lists all 1 and 2-gene models capable of distinguishing between subjects with breast cancer and melanoma (active disease, stages 2-4) with at least 75% accuracy. Table C2a lists all 1 and 2-gene models capable of distinguishing between subjects with breast cancer and ovarian cancer with at least 75% accuracy. Table C3a lists all 1 and 2-gene models capable of distinguishing between subjects with cervical cancer and breast cancer with at least 75% accuracy. Table C4a lists all 1 and 2-gene models capable of distinguishing between subjects with cervical cancer and colon cancer with at least 75% accuracy. Table C5a lists all 1 and 2-gene models capable of distinguishing between subjects with cervical cancer and melanoma (active disease, stages 2-4) with at least 75% accuracy. Table C6a lists all 2-gene models capable of distinguishing between subjects with cervical cancer and ovarian cancer with at least 75% accuracy. Table C7a lists all 1 and 2-gene models capable of distinguishing between subjects with colon cancer and melanoma (active disease, stages 2-4) with at least 75% accuracy. Table C8a lists all 1 and 2-gene models capable of distinguishing between subjects with lung cancer and breast cancer with at least 75% accuracy. Table C9a lists all 1 and 2-gene models capable of distinguishing between subjects with lung cancer and cervical cancer with at least 75% accuracy. Table C10a lists all 1 and 2-gene models capable of distinguishing between subjects with lung cancer and colon cancer with at least 75% accuracy. Table C11a lists all 1 and 2-gene models capable of distinguishing between subjects with lung cancer and melanoma (active disease, stages 2-4) with at least 75% accuracy. Table C12a lists all 2-gene models capable of distinguishing between subjects with lung cancer and ovarian cancer with at least 75% accuracy. Table C13a lists all 1 and 2-gene models capable of distinguishing between subjects with lung cancer and prostate cancer with at least 75% accuracy. Table C14a lists all 1 and 2-gene models capable of distinguishing between subjects with ovarian cancer and colon cancer with at least 75% accuracy. Table C15a lists all 1 and 2-gene models capable of distinguishing between subjects with ovarian cancer and melanoma (active disease, stages 2-4) with at least 75% accuracy. Table C16a lists all 1 and 2-gene models capable of distinguishing between subjects with prostate cancer and colon cancer with at least 75% accuracy. Table C17a lists all 1 and 2-gene models capable of distinguishing between subjects with prostate cancer and melanoma (active disease, stages 2-4) with at least 75% accuracy.

### Design of Assays

Typically, a sample is run through a panel in replicates of three for each target gene (assay); that is, a sample is divided into aliquots and for each aliquot the concentrations of each constituent in a Gene Expression Panel (Precision Profile™) is measured. From over thousands of constituent assays, with each assay conducted in triplicate, an average coefficient of variation was found (standard deviation/average)*100, of less than 2 percent among the normalized ΔCt measurements for each assay (where normalized quantitation of the target mRNA is determined by the difference in threshold cycles between the internal control (e.g., an endogenous marker such as 18S rRNA, or an exogenous marker) and the gene of interest. This is a measure called “intra-assay variability”. Assays have also been conducted on different occasions using the same sample material. This is a measure of “inter-assay variability”. Preferably, the average coefficient of variation of intra-assay variability or inter-assay variability is less than 20%, more preferably less than 10%, more preferably less than 5%, more preferably less than 4%, more preferably less than 3%, more preferably less than 2%, and even more preferably less than 1%.

It has been determined that it is valuable to use the quadruplicate or triplicate test results to identify and eliminate data points that are statistical “outliers”; such data points are those that differ by a percentage greater, for example, than 3% of the average of all three or four values. Moreover, if more than one data point in a set of three or four is excluded by this procedure, then all data for the relevant constituent is discarded.

### Measurement of Gene Expression for a Constituent in the Panel

For measuring the amount of a particular RNA in a sample, methods known to one of ordinary skill in the art were used to extract and quantify transcribed RNA from a sample with respect to a constituent of a Gene Expression Panel (Precision Profile™). (See detailed protocols below. Also see PCT application publication number WO 98/24935 herein incorporated by reference for RNA analysis protocols). Briefly, RNA is extracted from a sample such as any tissue, body fluid, cell (e.g., circulating tumor cell) or culture medium in which a population of cells of a subject might be growing. For example, cells may be lysed and RNA eluted in a suitable solution in which to conduct a DNAse reaction. Subsequent to RNA extraction, first strand synthesis may be performed using a reverse transcriptase. Gene amplification, more specifically quantitative PCR assays, can then be conducted and the gene of interest calibrated against an internal marker such as 18S rRNA (Hirayama et al., Blood 92, 1998: 46-52). Any other endogenous marker can be used, such as 28S-25S rRNA and 5S rRNA. Samples are measured in multiple replicates, for example, 3 replicates. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, quantitation of the reporter signal for an internal marker generated by the exponential increase of amplified product may also be used. Amplification of the target template may be accomplished by isothermic gene amplification strategies or by gene amplification by thermal cycling such as PCR.

It is desirable to obtain a definable and reproducible correlation between the amplified target or reporter signal, i.e., internal marker, and the concentration of starting templates. It has been discovered that this objective can be achieved by careful attention to, for example, consistent primer-template ratios and a strict adherence to a narrow permissible level of experimental amplification efficiencies (for example 80.0 to 100%+/−5% relative efficiency, typically 90.0 to 100%+/−5% relative efficiency, more typically 95.0 to 100%+/−2%, and most typically 98 to 100%+/−1% relative efficiency). In determining gene expression levels with regard to a single Gene Expression Profile, it is necessary that all constituents of the panels, including endogenous controls, maintain similar amplification efficiencies, as defined herein, to permit accurate and precise relative measurements for each constituent. Amplification efficiencies are regarded as being “substantially similar”, for the purposes of this description and the following claims, if they differ by no more than approximately 10%, preferably by less than approximately 5%, more preferably by less than approximately 3%, and more preferably by less than approximately 1%. Measurement conditions are regarded as being “substantially repeatable, for the purposes of this description and the following claims, if they differ by no more than approximately +/−10% coefficient of variation (CV), preferably by less than approximately +/−5% CV, more preferably +/−2% CV. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria directly, in such a manner as to compensate for errors, so that the criteria are satisfied after suitable adjustment of assay results.

In practice, tests are run to assure that these conditions are satisfied. For example, the design of all primer-probe sets are done in house, experimentation is performed to determine which set gives the best performance. Even though primer-probe design can be enhanced using computer techniques known in the art, and notwithstanding common practice, it has been found that experimental validation is still useful. Moreover, in the course of experimental validation, the selected primer-probe combination is associated with a set of features:

The reverse primer should be complementary to the coding DNA strand. In one embodiment, the primer should be located across an intron-exon junction, with not more than four bases of the three-prime end of the reverse primer complementary to the proximal exon. (If more than four bases are complementary, then it would tend to competitively amplify genomic DNA.)

In an embodiment of the invention, the primer probe set should amplify cDNA of less than 110 bases in length and should not amplify, or generate fluorescent signal from, genomic DNA or transcripts or cDNA from related but biologically irrelevant loci.

A suitable target of the selected primer probe is first strand cDNA, which in one embodiment may be prepared from whole blood as follows:

(a) Use of Whole Blood for Ex Vivo Assessment of a Biological Condition

Human blood is obtained by venipuncture and prepared for assay. The aliquots of heparinized, whole blood are mixed with additional test therapeutic compounds and held at 37° C. in an atmosphere of 5% CO₂ for 30 minutes. Cells are lysed and nucleic acids, e.g., RNA, are extracted by various standard means.

Nucleic acids, RNA and or DNA, are purified from cells, tissues or fluids of the test population of cells. RNA is preferentially obtained from the nucleic acid mix using a variety of standard procedures (or RNA Isolation Strategies, pp. 55-104, in RNA Methodologies, A laboratory guide for isolation and characterization, 2nd edition, 1998, Robert E. Farrell, Jr., Ed., Academic Press), in the present using a filter-based RNA isolation system from Ambion (RNAqueous™, Phenol-free Total RNA Isolation Kit, Catalog #1912, version 9908; Austin, Tex.).

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples (see, for example, RT PCR, Chapter 15 in RNA Methodologies, A Laboratory Guide for Isolation and Characterization, 2nd edition, 1998, Robert E. Farrell, Jr., Ed., Academic Press; or Chapter 22 pp. 143-151, RNA Isolation and Characterization Protocols, Methods in Molecular Biology, Volume 86, 1998, R. Rapley and D. L. Manning Eds., Human Press, or Chapter 14 Statistical refinement of primer design parameters; or Chapter 5, pp. 55-72, PCR Applications: protocols for functional genomics, M. A. Innis, D. H. Gelfand and J. J. Sninsky, Eds., 1999, Academic Press). Amplifications are carried out in either isothermic conditions or using a thermal cycler (for example, a ABI 9600 or 9700 or 7900 obtained from Applied Biosystems, Foster City, Calif.; see Nucleic acid detection methods, pp. 1-24, in Molecular Methods for Virus Detection, D. L. Wiedbrauk and D. H., Farkas, Eds., 1995, Academic Press). Amplified nucleic acids are detected using fluorescent-tagged detection oligonucleotide probes (see, for example, Taqman™ PCR Reagent Kit, Protocol, part number 402823, Revision A, 1996, Applied Biosystems, Foster City Calif.) that are identified and synthesized from publicly known databases as described for the amplification primers.

For example, without limitation, amplified cDNA is detected and quantified using detection systems such as the ABI Prism® 7900 Sequence Detection System (Applied Biosystems (Foster City, Calif.)), the Cepheid SmartCycler® and Cepheid GeneXpert® Systems, the Fluidigm BioMark™ System, and the Roche LightCycler® 480 Real-Time PCR System. Amounts of specific RNAs contained in the test sample can be related to the relative quantity of fluorescence observed (see for example, Advances in Quantitative PCR Technology: 5′ Nuclease Assays, Y. S. Lie and C. J. Petropolus, Current Opinion in Biotechnology, 1998, 9:43-48, or Rapid Thermal Cycling and PCR Kinetics, pp. 211-229, chapter 14 in PCR applications: protocols for functional genomics, M. A. Innis, D. H. Gelfand and J. J. Sninsky, Eds., 1999, Academic Press). Examples of the procedure used with several of the above-mentioned detection systems are described below. In some embodiments, these procedures can be used for both whole blood RNA and RNA extracted from cultured cells (e.g., without limitation, CTCs, and CECs). In some embodiments, any tissue, body fluid, or cell(s) (e.g., circulating tumor cells (CTCs) or circulating endothelial cells (CECs)) may be used for ex vivo assessment of a biological condition affected by an agent. Methods herein may also be applied using proteins where sensitive quantitative techniques, such as an Enzyme Linked ImmunoSorbent Assay (ELISA) or mass spectroscopy, are available and well-known in the art for measuring the amount of a protein constituent (see WO 98/24935 herein incorporated by reference).

An example of a procedure for the synthesis of first strand cDNA for use in PCR amplification is as follows:

Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent).

Methods

1. Place RNase Inhibitor and MultiScribe Reverse Transcriptase on ice immediately. All other reagents can be thawed at room temperature and then placed on ice.

2. Remove RNA samples from −80oC freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| 1 reaction (mL) | 11X, e.g. 10 samples (μL) | |
|---|---|---|
| 10X RT Buffer | 10.0 | 110.0 |
| 25 mM MgCl₂ | 22.0 | 242.0 |
| dNTPs | 20.0 | 220.0 |
| Random Hexamers | 5.0 | 55.0 |
| RNAse Inhibitor | 2.0 | 22.0 |
| Reverse Transcriptase | 2.5 | 27.5 |
| Water | 18.5 | 203.5 |
| Total: | 80.0 | 880.0 (80 μL per sample) |

4. Bring each RNA sample to a total volume of 20 μL in a 1.5 mL microcentrifuge tube (for example, RNA, remove 10 μL RNA and dilute to 20 μL with RNase/DNase free water, for whole blood RNA use 20 μL total RNA) and add 80 μL RT reaction mix from step 5, 2, 3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and (β-actin.

Following the synthesis of first strand cDNA, one particular embodiment of the approach for amplification of first strand cDNA by PCR, followed by detection and quantification of constituents of a Gene Expression Panel (Precision Profile™) is performed using the ABI Prism® 7900 Sequence Detection System as follows:

Materials

1. 20× Primer/Probe Mix for each gene of interest.

2. 20× Primer/Probe Mix for 18S endogenous control.

3. 2× Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism® 7700 or 7900 Sequence Detector.

Methods

1. Make stocks of each Primer/Probe mix containing the Primer/Probe for the gene of interest, Primer/Probe for 18S endogenous control, and 2×PCR Master Mix as follows. Make sufficient excess to allow for pipetting error e.g., approximately 10% excess. The following example illustrates a typical set up for one gene with quadruplicate samples testing two conditions (2 plates).

| | 1X (1 well) (μL) |
|---|---|
| 2X Master Mix | 7.5 |
| 20X 18S Primer/Probe Mix | 0.75 |
| 20X Gene of interest Primer/Probe Mix | 0.75 |
| Total | 9.0 |

2. Make stocks of cDNA targets by diluting 954 of cDNA into 20004 of water. The amount of cDNA is adjusted to give Ct values between 10 and 18, typically between 12 and 16.

3. Pipette 9 μL of Primer/Probe mix into the appropriate wells of an Applied Biosystems 384-Well Optical Reaction Plate.

4. Pipette 10 μL of cDNA stock solution into each well of the Applied Biosystems 384-Well Optical Reaction Plate.

5. Seal the plate with Applied Biosystems Optical Caps, or optical-clear film.

6. Analyze the plate on the ABI Prism® 7900 Sequence Detector.

In another embodiment of the invention, the use of the primer probe with the first strand cDNA as described above to permit measurement of constituents of a Gene Expression Panel (Precision Profile™) is performed using a QPCR assay on Cepheid SmartCycler® and GeneXpert® Instruments as follows:
I. To run a QPCR assay in duplicate on the Cepheid SmartCycler® instrument containing three target genes and one reference gene, the following procedure should be followed.

A. With 20× Primer/Probe Stocks.

Materials
1. SmartMix™-HM lyophilized Master Mix.
2. Molecular grade water.
3. 20× Primer/Probe Mix for the 18S endogenous control gene. The endogenous control gene will be dual labeled with VIC-MGB or equivalent.
4. 20× Primer/Probe Mix for each for target gene one, dual labeled with FAM-BHQ1 or equivalent.
5. 20× Primer/Probe Mix for each for target gene two, dual labeled with Texas Red-BHQ2 or equivalent.
6. 20× Primer/Probe Mix for each for target gene three, dual labeled with Alexa 647-BHQ3 or equivalent.
7. Tris buffer, pH 9.0
8. cDNA transcribed from RNA extracted from sample.
9. SmartCycler® 25 μL tube.
10. Cepheid SmartCycler® instrument.

Methods
1. For each cDNA sample to be investigated, add the following to a sterile 650 μL tube.

| | | |
|---|---|---|
| SmartMix ™-HM lyophilized Master Mix | 1 | bead |
| 20X 18S Primer/Probe Mix | 2.5 | μL |
| 20X Target Gene 1 Primer/Probe Mix | 2.5 | μL |
| 20X Target Gene 2 Primer/Probe Mix | 2.5 | μL |
| 20X Target Gene 3 Primer/Probe Mix | 2.5 | μL |
| Tris Buffer, pH 9.0 | 2.5 | μL |
| Sterile Water | 34.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.

B. With Lyophilized SmartBeads™.

Materials
1. SmartMix™-HM lyophilized Master Mix.
2. Molecular grade water.
3. SmartBeads™ containing the 18S endogenous control gene dual labeled with VIC-MGB or equivalent, and the three target genes, one dual labeled with FAM-BHQ1 or equivalent, one dual labeled with Texas Red-BHQ2 or equivalent and one dual labeled with Alexa 647-BHQ3 or equivalent.
4. Tris buffer, pH 9.0
5. cDNA transcribed from RNA extracted from sample.
6. SmartCycler® 25 μL tube.
7. Cepheid SmartCycler® instrument.

Methods
1. For each cDNA sample to be investigated, add the following to a sterile 650 μL tube.

| | | |
|---|---|---|
| SmartMix ™-HM lyophilized Master Mix | 1 | bead |
| SmartBead ™ containing four primer/probe sets | 1 | bead |
| Tris Buffer, pH 9.0 | 2.5 | μL |
| Sterile Water | 44.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler®tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.
II. To run a QPCR assay on the Cepheid GeneXpert® instrument containing three target genes and one reference gene, the following procedure should be followed. Note that to do duplicates, two self contained cartridges need to be loaded and run on the GeneXpert® instrument.

Materials
1. Cepheid GeneXpert® self contained cartridge preloaded with a lyophilized SmartMix™-HM master mix bead and a lyophilized SmartBead™ containing four primer/probe sets.
2. Molecular grade water, containing Tris buffer, pH 9.0.
3. Extraction and purification reagents.
4. Clinical sample (whole blood, RNA, etc.)
5. Cepheid GeneXpert® instrument.

Methods
1. Remove appropriate GeneXpert® self contained cartridge from packaging.
2. Fill appropriate chamber of self contained cartridge with molecular grade water with Tris buffer, pH 9.0.
3. Fill appropriate chambers of self contained cartridge with extraction and purification reagents.
4. Load aliquot of clinical sample into appropriate chamber of self contained cartridge.
5. Seal cartridge and load into GeneXpert® instrument.
6. Run the appropriate extraction and amplification protocol on the GeneXpert® and analyze the resultant data.

In yet another embodiment of the invention, the use of the primer probe with the first strand cDNA as described above to permit measurement of constituents of a Gene Expression Panel (Precision Profile™) is performed using a QPCR assay on the Roche LightCycler® 480 Real-Time PCR System as follows:

Materials
1. 20× Primer/Probe stock for the 18S endogenous control gene. The endogenous control gene may be dual labeled with either VIC-MGB or VIC-TAMRA.
2. 20× Primer/Probe stock for each target gene, dual labeled with either FAM-TAMRA or FAM-BHQ1.
3. 2× LightCycler® 490 Probes Master (master mix).
4. 1× cDNA sample stocks transcribed from RNA extracted from samples.
5. 1× TE buffer, pH 8.0.
6. LightCycler® 480 384-well plates.
7. Source MDx 24 gene Precision Profile™ 96-well intermediate plates.
8. RNase/DNase free 96-well plate.
9. 1.5 mL microcentrifuge tubes.
10. Beckman/Coulter Biomek® 3000 Laboratory Automation Workstation.
11. Velocity11 Bravo™ Liquid Handling Platform.
12. LightCycler® 480 Real-Time PCR System.

Methods
1. Remove a Source MDx 24 gene Precision Profile™ 96-well intermediate plate from the freezer, thaw and spin in a plate centrifuge.
2. Dilute four (4) 1× cDNA sample stocks in separate 1.5 mL microcentrifuge tubes with the total final volume for each of 540 μL.
3. Transfer the 4 diluted cDNA samples to an empty RNase/DNase free 96-well plate using the Biomek® 3000 Laboratory Automation Workstation.
4. Transfer the cDNA samples from the cDNA plate created in step 3 to the thawed and centrifuged Source MDx 24 gene Precision Profile™ 96-well intermediate plate using Biomek® 3000 Laboratory Automation Workstation. Seal the plate with a foil seal and spin in a plate centrifuge.
5. Transfer the contents of the cDNA-loaded Source MDx 24 gene Precision Profile™ 96-well intermediate plate to a new LightCycler® 480 384-well plate using the Bravo™ Liquid Handling Platform. Seal the 384-well plate with a LightCycler® 480 optical sealing foil and spin in a plate centrifuge for 1 minute at 2000 rpm.
6. Place the sealed in a dark 4° C. refrigerator for a minimum of 4 minutes.
7. Load the plate into the LightCycler® 480 Real-Time PCR System and start the LightCycler® 480 software. Chose the appropriate run parameters and start the run.
8. At the conclusion of the run, analyze the data and export the resulting CP values to the database.

In some instances, target gene FAM measurements may be beyond the detection limit of the particular platform instrument used to detect and quantify constituents of a Gene Expression Panel (Precision Profile™). To address the issue of “undetermined” gene expression measures as lack of expression for a particular gene, the detection limit may be reset and the “undetermined” constituents may be “flagged”. For example without limitation, the ABI Prism® 7900HT Sequence Detection System reports target gene FAM measurements that are beyond the detection limit of the instrument (>40 cycles) as “undetermined”. Detection Limit Reset is performed when at least 1 of 3 target gene FAM C_{T} replicates are not detected after 40 cycles and are designated as “undetermined”. “Undetermined” target gene FAM C_{T} replicates are re-set to 40 and flagged. C_{T} normalization (ΔC_{T}) and relative expression calculations that have used re-set FAM C_{T} values are also flagged.

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a biological condition or agent. Baseline profile data sets may be stored in libraries and classified in a number of cross-referential ways. One form of classification may rely on the characteristics of the panels from which the data sets are derived. Another form of classification may be by particular biological condition, e.g., breast, ovarian, cervical, prostate, lung, skin or colon cancer cancer. The concept of a biological condition encompasses any state in which a cell or population of cells may be found at any one time. This state may reflect geography of samples, sex of subjects or any other discriminator. Some of the discriminators may overlap. The libraries may also be accessed for records associated with a single subject or particular clinical trial. The classification of baseline profile data sets may further be annotated with medical information about a particular subject, a medical condition, and/or a particular agent.

### Calibrated Data

Given the repeatability achieved in measurement of gene expression, described above in connection with “Gene Expression Panels” (Precision Profiles™) and “gene amplification”, it was concluded that where differences occur in measurement under such conditions, the differences are attributable to differences in biological condition. Thus, it has been found that calibrated profile data sets are highly reproducible in samples taken from the same individual under the same conditions. Similarly, it has been found that calibrated profile data sets are reproducible in samples that are repeatedly tested.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within 20%, and typically within 10%. In accordance with embodiments of the invention, a pattern of increasing, decreasing and no change in relative gene expression from each of a plurality of gene loci examined in the Gene Expression Panel (Precision Profile™) may be used to prepare a calibrated profile set that is informative with regards to a biological condition, e.g. cancer type or cancer stage.

The numerical data obtained from quantitative gene expression and numerical data from calibrated gene expression relative to a baseline profile data set may be stored in databases or digital storage mediums and may be retrieved for purposes including managing patient health care. The data may be transferred in physical or wireless networks via the World Wide Web, email, or interne access site for example or by hard copy so as to be collected and pooled from distant geographic sites.

The method also includes producing a calibrated profile data set for the panel, wherein each member of the calibrated profile data set is a function of a corresponding member of the first profile data set and a corresponding member of a baseline profile data set for the panel, and wherein the baseline profile data set is related to the one type of cancer to be evaluated, with the calibrated profile data set being a comparison between the first profile data set and the baseline profile data set, thereby providing evaluation of the type of cancer.

In yet other embodiments, the function is a mathematical function and is other than a simple difference, including a second function of the ratio of the corresponding member of first profile data set to the corresponding member of the baseline profile data set, or a logarithmic function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data.

Because the data is in a universal format, data handling may readily be done with a computer. The data is organized so as to provide an output optionally corresponding to a graphical representation of a calibrated data set.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The graphical representation of calibrated profile data sets pertaining to a product such as a drug provides an opportunity for standardizing a product by means of the calibrated profile, more particularly a signature profile. The profile may be used as a feature with which to demonstrate relative efficacy, differences in mechanisms of actions, etc. compared to other drugs approved for similar or different uses.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set.

The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

In other embodiments, a clinical indicator may be used to assess the cancer of the relevant set of subjects by interpreting the calibrated profile data set in the context of at least one other clinical indicator, wherein the at least one other clinical indicator is selected from the group consisting of blood chemistry, X-ray or other radiological or metabolic imaging technique, molecular markers in the blood, other chemical assays, and physical findings.

### Index Construction

In combination, (i) the remarkable consistency of Gene Expression Profiles with respect to a biological condition across a population or set of subject or samples, or across a population of cells and (ii) the use of procedures that provide substantially reproducible measurement of constituents in a Gene Expression Panel (Precision Profile™) giving rise to a Gene Expression Profile, under measurement conditions wherein specificity and efficiencies of amplification for all constituents of the panel are substantially similar, make possible the use of an index that characterizes a Gene Expression Profile, and which therefore provides a measurement of the particular cancer

An index may be constructed using an index function that maps values in a Gene Expression Profile into a single value that is pertinent to the biological condition at hand. The values in a Gene Expression Profile are the amounts of each constituent of the Gene Expression Panel (Precision Profile™). These constituent amounts form a profile data set, and the index function generates a single value—the index—from the members of the profile data set.

The index function may conveniently be constructed as a linear sum of terms, each term being what is referred to herein as a “contribution function” of a member of the profile data set. For example, the contribution function may be a constant times a power of a member of the profile data set. So the index function would have the form

I=ΣCiMi^{P(i)},

where I is the index, Mi is the value of the member i of the profile data set, Ci is a constant, and P(i) is a power to which Mi is raised, the sum being formed for all integral values of i up to the number of members in the data set. We thus have a linear polynomial expression. The role of the coefficient Ci for a particular gene expression specifies whether a higher ΔCt value for this gene either increases (a positive Ci) or decreases (a lower value) the likelihood of cancer, the ΔCt values of all other genes in the expression being held constant.

The values Ci and P(i) may be determined in a number of ways, so that the index I is informative of the pertinent biological condition. One way is to apply statistical techniques, such as latent class modeling, to the profile data sets to correlate clinical data or experimentally derived data, or other data pertinent to the biological condition. In this connection, for example, may be employed the software from Statistical Innovations, Belmont, Mass., called Latent Gold®. Alternatively, other simpler modeling techniques may be employed in a manner known in the art.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition, clinical indicator, medication, physical activity, body mass, and environmental exposure.

As an example, the index can be constructed, in relation to a normative Gene Expression Profile for a population or set of cancer subjects, in such a way that a reading of approximately 1 characterizes normative Gene Expression Profiles of subjects with a particular cancer. Let us further assume that the biological condition that is the subject of the index is cancer; a reading of 1 in this example thus corresponds to a Gene Expression Profile that matches the norm for subject with that particular cancer. A substantially higher reading then may identify a subject experiencing a different type of cancer. The use of 1 as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since it was determined that Gene Expression Profile values (and accordingly constructed indices based on them) tend to be normally distributed, the 0-centered index constructed in this manner is highly informative. It therefore facilitates use of the index in diagnosis of disease.

As another embodiment of the invention, an index function I of the form

I=C₀+ΣCᵢM₁ᵢ^{P1(i)}M₂ᵢ^{P2(i)},

can be employed, where M₁ and M₂ are values of the member i of the profile data set, Cᵢ is a constant determined without reference to the profile data set, and P1 and P2 are powers to which M₁ and M₂ are raised. The role of P1(i) and P2(i) is to specify the specific functional form of the quadratic expression, whether in fact the equation is linear, quadratic, contains cross-product terms, or is constant. For example, when P1=P2=0, the index function is simply the sum of constants; when P1=1 and P2=0, the index function is a linear expression; when P1=P2=1, the index function is a quadratic expression.

The constant C₀ serves to calibrate this expression to the biological population of interest that is characterized by having a particular type of cancer. In this embodiment, when the index value equals 0, the odds are 50:50 of the subject having one type of cancer vs another type of cancer. More generally, the predicted odds of the subject having one type of cancer is [exp(Iᵢ)], and therefore the predicted probability of having another type of cancer is [exp(Iᵢ)]/[1+exp(Iᵢ)]. Thus, when the index exceeds 0, the predicted probability that a subject has the particular type of cancer is higher than 0.5, and when it falls below 0, the predicted probability is less than 0.5.

The value of C₀ may be adjusted to reflect the prior probability of being in this population based on known exogenous risk factors for the subject. In an embodiment where C₀ is adjusted as a function of the subject's risk factors, where the subject has prior probability pᵢ of having a particular cancer based on such risk factors, the adjustment is made by increasing (decreasing) the unadjusted C₀ value by adding to C₀ the natural logarithm of the following ratio: the prior odds of having a particular cancer taking into account the risk factors/the overall prior odds of having a particular cancer without taking into account the risk factors. Risk factors include risk factors associated with a particular cancer based upon the sex of the individual. For example the risk factor of a female subject developing prostate cancer is zero. Similarly, the risk factor is a male subject having ovarian cancer is zero.

### Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between a subject having one type of cancer versus another type cancer is based on whether the subjects have an “effective amount” or a “significant alteration” in the levels of a cancer associated gene. By “effective amount” or “significant alteration”, it is meant that the measurement of an appropriate number of cancer associated gene (which may be one or more) is different than the predetermined cut-off point (or threshold value) for that cancer associated gene and therefore indicates that the subject has the cancer for which the cancer associated gene(s) is a determinant.

The difference in the level of cancer associated gene(s) between normal and abnormal is preferably statistically significant. As noted below, and without any limitation of the invention, achieving statistical significance, and thus the preferred analytical and clinical accuracy, generally but not always requires that combinations of several cancer associated gene(s) be used together in panels and combined with mathematical algorithms in order to achieve a statistically significant cancer associated gene index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay (such as the test of the invention for determining an effective amount or a significant alteration of cancer associated gene(s), which thereby indicates the presence of a cancer in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, desirably at least 0.775, more desirably at least 0.800, preferably at least 0.825, more preferably at least 0.850, and most preferably at least 0.875.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing cancer, and the bottom quartile comprising the group of subjects having the lowest relative risk for developing cancer. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease. Often such lower diagnostic accuracy tests must be combined with additional parameters in order to derive meaningful clinical thresholds for therapeutic intervention, as is done with the aforementioned global risk assessment indices.

A health economic utility function is yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as those at risk for having a bone fracture) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the cancer associated gene(s) of the invention allows for one of skill in the art to use the cancer associated gene(s) to identify, diagnose, or prognose subjects with a pre-determined level of predictability and performance.

Results from the cancer associated gene(s) indices thus derived can then be validated through their calibration with actual results, that is, by comparing the predicted versus observed rate of disease in a given population, and the best predictive cancer associated gene(s) selected for and optimized through mathematical models of increased complexity. Many such formula may be used; beyond the simple non-linear transformations, such as logistic regression, of particular interest in this use of the present invention are structural and synactic classification algorithms, and methods of risk index construction, utilizing pattern recognition features, including established techniques such as the Kth-Nearest Neighbor, Boosting, Decision Trees, Neural Networks, Bayesian Networks, Support Vector Machines, and Hidden Markov Models, as well as other formula described herein.

Furthermore, the application of such techniques to panels of multiple cancer associated gene(s) is provided, as is the use of such combination to create single numerical “risk indices” or “risk scores” encompassing information from multiple cancer associated gene(s) inputs. Individual B cancer associated gene(s) may also be included or excluded in the panel of cancer associated gene(s) used in the calculation of the cancer associated gene(s) indices so derived above, based on various measures of relative performance and calibration in validation, and employing through repetitive training methods such as forward, reverse, and stepwise selection, as well as with genetic algorithm approaches, with or without the use of constraints on the complexity of the resulting cancer associated gene(s) indices.

The above measurements of diagnostic accuracy for cancer associated gene(s) are only a few of the possible measurements of the clinical performance of the invention. It should be noted that the appropriateness of one measurement of clinical accuracy or another will vary based upon the clinical application, the population tested, and the clinical consequences of any potential misclassification of subjects. Other important aspects of the clinical and overall performance of the invention include the selection of cancer associated gene(s) so as to reduce overall cancer associated gene(s) variability (whether due to method (analytical) or biological (pre-analytical variability, for example, as in diurnal variation), or to the integration and analysis of results (post-analytical variability) into indices and cut-off ranges), to assess analyte stability or sample integrity, or to allow the use of differing sample matrices amongst blood, cells, serum, plasma, urine, etc.

### Kits

The invention also includes an cancer detection reagent, i.e., nucleic acids that specifically identify one or more cancer or condition related to cancer nucleic acids (e.g., any gene listed in Tables A-C, oncogenes, tumor suppression genes, tumor progression genes, angiogenesis genes and lymphogenesis genes; sometimes referred to herein as cancer associated genes or cancer associated constituents) by having homologous nucleic acid sequences, such as oligonucleotide sequences, complementary to a portion of the cancer genes nucleic acids or antibodies to proteins encoded by the cancer gene nucleic acids packaged together in the form of a kit. The oligonucleotides can be fragments of the cancer genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label. Instructions (i.e., written, tape, VCR, CD-ROM, etc.) for carrying out the assay may be included in the kit. The assay may for example be in the form of PCR, a Northern hybridization or a sandwich ELISA, as known in the art.

For example, cancer gene detection reagents can be immobilized on a solid matrix such as a porous strip to form at least one cancer gene detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of cancer genes present in the sample. The detection sites may be configured in any suitably detectable shape and are typically in the shape of a bar or dot spanning the width of a test strip.

Alternatively, cancer detection genes can be labeled (e.g., with one or more fluorescent dyes) and immobilized on lyophilized beads to form at least one cancer gene detection site. The beads may also contain sites for negative and/or positive controls. Upon addition of the test sample, the number of sites displaying a detectable signal provides a quantitative indication of the amount of cancer genes present in the sample.

Alternatively, the kit contains a nucleic acid substrate array comprising one or more nucleic acid sequences. The nucleic acids on the array specifically identify one or more nucleic acid sequences represented by cancer genes (see Tables A-C). In various embodiments, the expression of 1, 2, 3, 4, 5, 6, 7, 8, 9, 10, 15, 20, 25, 40 or 50 or more of the sequences represented by cancer genes (see Tables A-C) can be identified by virtue of binding to the array. The substrate array can be on, i.e., a solid substrate, i.e., a “chip” as described in U.S. Pat. No. 5,744,305. Alternatively, the substrate array can be a solution array, i.e., Luminex, Cyvera, Vitra and Quantum Dots' Mosaic.

The skilled artisan can routinely make antibodies, nucleic acid probes, i.e., oligonucleotides, aptamers, siRNAs, antisense oligonucleotides, against any of the cancer genes listed in Tables A-C.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1

### Patient Populations

RNA was isolated using the PAXgene System from blood samples obtained from the following groups of cancer patients described below. These RNA samples were used for the gene expression analysis studies described in Examples 3-5.

### Melanoma:

Blood samples obtained from a total of 87 subjects suffering from melanoma. The study participants included male and female subjects, each 18 years or older and able to provide consent. The study population included subjects having Stage 1, Stage 2, Stage 3, and Stage 4 melanoma, and subjects having either active (i.e., clinical evidence of disease, and including subjects that had blood drawn within 2-3 weeks post resection even though clinical evidence of disease was not necessarily present after resection) or inactive disease (i.e., no clinical evidence of disease). Staging was evaluated and tracked according to tumor thickness and ulceration, spread to lymph nodes, and metastasis to distant organs. RNA samples from all melanoma subjects described (i.e., stages 1-4, active and inactive disease) were used to generate the logistic regression gene-models, as indicated in Examples 3-5 below.

### Lung Cancer

Blood samples were obtained from 49 subjects suffering from lung cancer. The inclusion criteria were as follows: each of the subjects had defined, newly diagnosed disease, the blood samples were obtained prior to initiation of any treatment for lung cancer, and each subject in the study was 18 years or older, and able to provide consent. The following criteria were used to exclude subjects from the study: any treatment with immunosuppressive drugs, corticosteroids or investigational drugs; diagnosis of acute and chronic infectious diseases (renal or chest infections, previous TB, HIV infection or AIDS, or active cytomegalovirus); symptoms of severe progression or uncontrolled renal, hepatic, hematological, gastrointestinal, endocrine, pulmonary, neurologic, or cerebral disease; and pregnancy.

Of the 49 newly diagnosed lung cancer subjects from which blood samples were obtained, 1 subject was diagnosed with small cell carcinoma and the remaining 48 subjects were diagnosed with non-small cell carcinoma; 1 subject was diagnosed with stage 1 lung cancer, 18 subjects were diagnosed with stage 2 lung cancer, and 30 subjects were diagnosed with stage 3 lung cancer; 41 subjects were smokers, and the remaining 8 subjects were non-smokers; 7 of the subjects were female, and the remaining 42 subjects were male. RNA samples from all lung cancer subjects described (i.e., all stages) were used to generate the logistic regression gene-models described in Examples 3-5 below.

### Colon Cancer

Blood samples were obtained from 23 subjects suffering from colon cancer. The inclusion criteria were as follows: each of the subjects had defined, newly diagnosed disease, the blood samples were obtained prior to initiation of any treatment for colon cancer, and each subject in the study was 18 years or older, and able to provide consent.

The following criteria were used to exclude subjects from the study: any treatment with immunosuppressive drugs, corticosteroids or investigational drugs; diagnosis of acute and chronic infectious diseases (renal or chest infections, previous TB, HIV infection or AIDS, or active cytomegalovirus); symptoms of severe progression or uncontrolled renal, hepatic, hematological, gastrointestinal, endocrine, pulmonary, neurological, or cerebral disease; and pregnancy.

### Prostate Cancer

Blood samples were obtained from 51 male subjects suffering from prostate cancer. The inclusion criteria were as follows: each of the subjects had ongoing prostate cancer or a history of previously treated prostate cancer, each subject in the study was 18 years or older, and able to provide consent. No exclusion criteria were used when screening participants.

Of the 40 prostate cancer subjects from which blood samples were obtained, 14 of the subjects had untreated localized prostate cancer (low, medium, or high risk) (cohort 1); 1 subject had rising PSA level after local therapy and prior to androgen deprivation therapy (cohort 2); 2 subjects had no detectable metastases, were on primary hormones, and in were in remission (cohort 3); 19 subjects had hormone or taxane refractory disease, with or without bone metastasis (cohort 4); and the disease status of 4 subjects was unknown (cohort 5). RNA samples from all prostate cancer subjects described (i.e., all cohorts) were used to generate the logistic regression gene-models described in Examples 3-5 below.

### Ovarian

Blood samples were obtained from 24 female subjects suffering from ovarian cancer.

The inclusion criteria were as follows: each of the subjects had defined, newly diagnosed disease, the blood samples were obtained prior to initiation of any treatment for ovarian cancer, and each subject in the study was 18 years or older, and able to provide consent.

The following criteria were used to exclude subjects from the study: any treatment with immunosuppressive drugs, corticosteroids or investigational drugs; diagnosis of acute and chronic infectious diseases (renal or chest infections, previous TB, HIV infection or AIDS, or active cytomegalovirus); symptoms of severe progression or uncontrolled renal, hepatic, hematological, gastrointestinal, endocrine, pulmonary, neurological, or cerebral disease; and pregnancy.

Of the 24 newly diagnosed ovarian cancer subjects from which blood samples were obtained, 8 subjects were diagnosed with Stage 1 ovarian cancer, 3 subjects were diagnosed with Stage 2 ovarian cancer, and 13 subjects were diagnosed with Stage 3 ovarian cancer. RNA samples from all ovarian cancer subjects described (i.e., all stages) were used to generate the logistic regression gene-models described in Examples 3-5 below.

### Breast Cancer

Blood samples were obtained from 49 female subjects suffering from breast cancer. The inclusion criteria were as follows: each of the subjects had defined, newly diagnosed disease, the blood samples were obtained prior to initiation of any treatment for breast cancer, and each subject in the study was 18 years or older, and able to provide consent.

The following criteria were used to exclude subjects from the study: any treatment with immunosuppressive drugs, corticosteroids or investigational drugs; diagnosis of acute and chronic infectious diseases (renal or chest infections, previous TB, HIV infection or AIDS, or active cytomegalovirus); symptoms of severe progression or uncontrolled renal, hepatic, hematological, gastrointestinal, endocrine, pulmonary, neurological, or cerebral disease; and pregnancy.

Of the 49 newly diagnosed breast cancer subjects from which blood samples were obtained, 2 subjects were diagnosed with Stage 0 (in situ) breast cancer, 17 subjects were diagnosed with Stage 1 breast cancer, 26 subjects were diagnosed with Stage 2 breast cancer, 1 subject was diagnosed with Stage 3 breast cancer, and 3 subjects were diagnosed with Stage 4 breast cancer. RNA samples from all breast cancer subjects described (i.e., all stages) were used to generate the logistic regression gene-models described in Examples 3-5 below.

### Cervical Cancer

Blood samples were obtained from a total of 24 female subjects suffering from cervical cancer. The inclusion criteria were as follows: each of the subjects had defined, newly diagnosed disease, the blood samples were obtained prior to initiation of any treatment for cervical cancer, and each subject in the study was 18 years or older, and able to provide consent.

The following criteria were used to exclude subjects from the study: any treatment with immunosuppressive drugs, corticosteroids or investigational drugs; diagnosis of acute and chronic infectious diseases (renal or chest infections, previous TB, HIV infection or AIDS, or active cytomegalovirus); symptoms of severe progression or uncontrolled renal, hepatic, hematological, gastrointestinal, endocrine, pulmonary, neurological, or cerebral disease; and pregnancy.

Of the 24 newly diagnosed cervical cancer subjects from which blood samples were obtained, 8 subjects were diagnosed with Stage 0 (in situ) cervical cancer, 13 subjects were diagnosed with Stage 1 cervical cancer, 1 subject was diagnosed with Stage 2 cervical cancer, and 2 subjects were diagnosed with Stage 3 cervical cancer. RNA samples from all cervical cancer subjects described (i.e., all cohorts) were used to generate the logistic regression gene-models described in Examples 3-5 below.

### Example 2

### Enumeration and Classification Methodology Based on Logistic Regression Models

### Introduction

The following methods were used to generate the 1, 2, and 3-gene models capable of distinguishing between subjects with diagnosed one type of cancer (including but not limited to skin, lung, colon, prostate, ovarian, cervical, or breast cancer), from another type of cancer (including but not limited to skin, lung, colon, prostate, ovarian, cervical or breast cancer), with at least 75% classification accuracy, described in Examples 3-5 below.

Given measurements on G genes from samples of N₁ subjects belonging to group 1 and N₂ members of group 2, the purpose was to identify models containing g<G genes which discriminate between the 2 groups. The groups might be such that subjects in group 1 may have disease A while those in group 2 may have disease B.

Specifically, parameters from a linear logistic regression model were estimated to predict a subject's probability of belonging to group 1 given his (her) measurements on the g genes in the model. After all the models were estimated (all G1-gene models were estimated, as well as all

$$( \begin{matrix}G \\ 2\end{matrix} ) = \frac{G * ( G - 1 )}{2} 2 \text{-} gene models ,$$

and all G3=G*(G−1)*(G−2)/6 3-gene models based on G genes (number of combinations taken 3 at a time from G)), they were evaluated using a 2-dimensional screening process. The first dimension employed a statistical screen (significance of incremental p-values) that eliminated models that were likely to overfit the data and thus may not validate when applied to new subjects. The second dimension employed a clinical screen to eliminate models for which the expected misclassification rate was higher than an acceptable level. As a threshold analysis, the gene models showing less than 75% discrimination between N₁ subjects belonging to group 1 and N₂ members of group 2 (i.e., misclassification of 25% or more of subjects in either of the 2 sample groups), and genes with incremental p-values that were not statistically significant, were eliminated.

### Methodological, Statistical and Computing Tools Used

The Latent GOLD program (Vermunt and Magidson, 2005) was used to estimate the logistic regression models. For efficiency in processing the models, the LG-Syntax™ Module available with version 4.5 of the program (Vermunt and Magidson, 2007) was used in batch mode, and all g-gene models associated with a particular dataset were submitted in a single run to be estimated. That is, all 1-gene models were submitted in a single run, all 2-gene models were submitted in a second run, etc.

### The Data

The data consists of ΔC_{T} values for each sample subject in each of the 2 groups (e.g., cancer subject A vs. cancer subject B on each of G(k) genes obtained from a particular class k of genes. For a given disease, separate analyses were performed based on inflammatory genes (k=1), human cancer general genes (k=2), and genes in the EGR family (k=3).

### Analysis Steps

The steps in a given analysis of the G(k) genes measured on N₁ subjects in group 1 and N₂ subjects in group 2 are as follows:
1) Eliminate low expressing genes: In some instances, target gene FAM measurements were beyond the detection limit (i.e., very high ΔC_{T} values which indicate low expression) of the particular platform instrument used to detect and quantify constituents of a Gene Expression Panel (Precision Profile™). To address the issue of “undetermined” gene expression measures as lack of expression for a particular gene, the detection limit was reset and the “undetermined” constituents were “flagged”, as previously described. C_{T} normalization (ΔC_{T}) and relative expression calculations that have used re-set FAM C_{T} values were also flagged. In some instances, these low expressing genes (i.e., re-set FAM C_{T} values) were eliminated from the analysis in step 1 if 50% or more ΔC_{T} values from either of the 2 groups were flagged. Although such genes were eliminated from the statistical analyses described herein, one skilled in the art would recognize that such genes may be relevant in a disease state.
2) Estimate logistic regression (logit) models predicting P(i)=the probability of being in group 1 for each subject i=1, 2, . . . , N₁+N₂. Since there are only 2 groups, the probability of being in group 2 equals 1−P(i). The maximum likelihood (ML) algorithm implemented in Latent GOLD 4.0 (Vermunt and Magidson, 2005) was used to estimate the model parameters. All 1-gene models were estimated first, followed by all 2-gene models and in cases where the sample sizes N₁ and N₂ were sufficiently large, all 3-gene models were estimated.
3) Screen out models that fail to meet the statistical or clinical criteria: Regarding the statistical criteria, models were retained if the incremental p-values for the parameter estimates for each gene (i.e., for each predictor in the model) fell below the cutoff point alpha=0.05. Regarding the clinical criteria, models were retained if the percentage of cases within each group (e.g., disease group A, and disease group B) that was correctly predicted to be in that group was at least 75%. For technical details, see the section “Application of the Statistical and Clinical Criteria to Screen Models”.
4) Each model yielded an index that could be used to rank the sample subjects. Such an index value could also be computed for new cases not included in the sample. See the section “Computing Model-based Indices for each Subject” for details on how this index was calculated.
5) A cutoff value somewhere between the lowest and highest index value was selected and based on this cutoff, subjects with indices above the cutoff were classified (predicted to be) in the disease group A, those below the cutoff were classified into disease group B. Based on such classifications, the percent of each group that is correctly classified was determined. See the section labeled “Classifying Subjects into Groups” for details on how the cutoff was chosen.
6) Among all models that survived the screening criteria (Step 3), an entropy-based R² statistic was used to rank the models from high to low, i.e., the models with the highest percent classification rate to the lowest percent classification rate. The top 5 such models are then evaluated with respect to the percent correctly classified and the one having the highest percentages was selected as the single “best” model. A discrimination plot was provided for the best model having an 85% or greater percent classification rate. For details on how this plot was developed, see the section “Discrimination Plots” below.

While there are several possible R² statistics that might be used for this purpose, it was determined that the one based on entropy was most sensitive to the extent to which a model yields clear separation between the 2 groups. Such sensitivity provides a model which can be used as a tool by a practitioner (e.g., primary care physician, oncologist, etc.) to ascertain the necessity of future screening or treatment options. For more detail on this issue, see the section labeled “Using R²Statistics to Rank Models” below.

### Computing Model-Based Indices for Each Subject

The model parameter estimates were used to compute a numeric value (logit, odds or probability) for each subject (i.e., disease A and disease B) in the sample. For illustrative purposes only, in an example of a 2-gene logit model for cancer containing the genes ALOX5 and S100A6, the following parameter estimates listed in Table A were obtained:

TABLE A

| | | |
|---|---|---|
| Cancer | alpha(1) | 18.37 |
| Reference | alpha(2) | −18.37 |
| Predictors | | |
| ALOX5 | beta(1) | −4.81 |
| S100A6 | beta(2) | 2.79 |

For a given subject with particular ΔC_{T} values observed for these genes, the predicted logit associated with cancer A vs. the reference group (e.g., cancer B) was computed as:

LOGIT(ALOX5,S100A6)=[alpha(1)−alpha(2)]+beta(1)*ALOX5+beta(2)*S100A6.

The predicted odds of having cancer A would be:

ODDS(ALOX5,S100A6)=exp[LOGIT(ALOX5,S100A6)]

and the predicted probability of belonging to the cancer A group is:

P(ALOX5,S100A6)=ODDS(ALOX5,S100A6)/[1+ODDS(ALOX5,S100A6)]

Note that the ML estimates for the alpha parameters were based on the relative proportion of the group sample sizes. Prior to computing the predicted probabilities, the alpha estimates may be adjusted to take into account the relative proportion in the population to which the model will be applied (for example, without limitation, the incidence of prostate cancer in the population of adult men in the U.S., the incidence of breast cancer in the population of adult women in the U.S., etc.)

Classifying Subjects into Groups

The “modal classification rule” was used to predict into which group a given case belongs. This rule classifies a case into the group for which the model yields the highest predicted probability. Using the same cancer example previously described (for illustrative purposes only), use of the modal classification rule would classify any subject having P>0.5 into the cancer A group, the others into the reference group (e.g., cancer B group). The percentage of all N₁ cancer subjects that were correctly classified were computed as the number of such subjects having P>0.5 divided by N₁. Similarly, the percentage of all N₂ reference (e.g., cancer B) subjects that were correctly classified were computed as the number of such subjects having P≦0.5 divided by N₂. Alternatively, a cutoff point P₀ could be used instead of the modal classification rule so that any subject i having P(i)>P₀ is assigned to the cancer A group, and otherwise to the reference group.

### Application of the Statistical and Clinical Criteria to Screen Models

### Clinical Screening Criteria

In order to determine whether a model met the clinical 75% correct classification criteria, the following approach was used:
A. All sample subjects were ranked from high to low by their predicted probability P (e.g., see Table B).
B. Taking P₀(i)=P(i) for each subject, one at a time, the percentage of group 1 and group 2 that would be correctly classified, P₁(i) and P₂(i) was computed.
C. The information in the resulting table was scanned and any models for which none of the potential cutoff probabilities met the clinical criteria (i.e., no cutoffs P₀(i) exist such that both P₁(i)>0.75 and P₂(i)>0.75) were eliminated. Hence, models that did not meet the clinical criteria were eliminated.

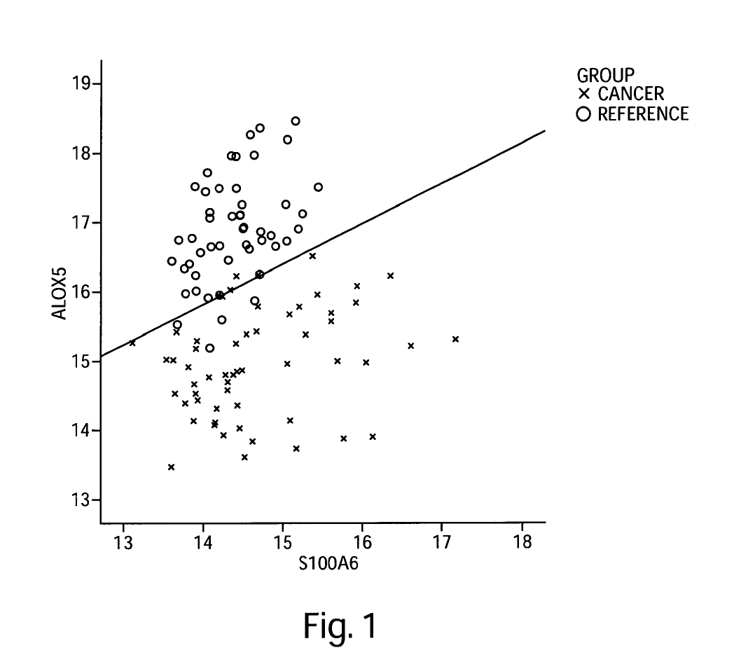

The example shown in Table B has many cut-offs that meet this criteria. For example, the cutoff P₀=0.4 yields correct classification rates of 92% for the reference group (e.g., Cancer B) and 93% for Cancer A subjects. A plot based on this cutoff is shown in FIG. 1 and described in the section “Discrimination Plots”.

### Statistical Screening Criteria

In order to determine whether a model met the statistical criteria, the following approach was used to compute the incremental p-value for each gene g=1, 2, . . . , G as follows:
i. Let LSQ(0) denote the overall model L-squared output by Latent GOLD for an unrestricted model.
ii. Let LSQ(g) denote the overall model L-squared output by Latent GOLD for the restricted version of the model where the effect of gene g is restricted to 0.
iii. With 1 degree of freedom, use a ‘components of chi-square’ table to determine the p-value associated with the LR difference statistic LSQ(g)−LSQ(0).
   Note that this approach required estimating g restricted models as well as 1 unrestricted model.

### Discrimination Plots

For a 2-gene model, a discrimination plot consisted of plotting the ΔC_{T} values for each subject in a scatterplot where the values associated with one of the genes served as the vertical axis, the other serving as the horizontal axis. Two different symbols were used for the points to denote whether the subject belongs to group 1 or 2.

A line was appended to a discrimination graph to illustrate how well the 2-gene model discriminated between the 2 groups. The slope of the line was determined by computing the ratio of the ML parameter estimate associated with the gene plotted along the horizontal axis divided by the corresponding estimate associated with the gene plotted along the vertical axis. The intercept of the line was determined as a function of the cutoff point. For the cancer example model based on the 2 genes ALOX5 and S100A6 shown in FIG. 1, the equation for the line associated with the cutoff of 0.4 is ALOX5=7.7+0.58*S100A6. This line provides correct classification rates of 93% and 92% (4 of 57 cancer subjects misclassified and only 4 of 50 reference subjects misclassified).

For a 3-gene model, a 2-dimensional slice defined as a linear combination of 2 of the genes was plotted along one of the axes, the remaining gene being plotted along the other axis. The particular linear combination was determined based on the parameter estimates. For example, if a 3^{rd} gene were added to the 2-gene model consisting of ALOX5 and S100A6 and the parameter estimates for ALOX5 and S100A6 were beta(1) and beta(2) respectively, the linear combination beta(1)*ALOX5+beta(2)*S100A6 could be used. This approach can be readily extended to the situation with 4 or more genes in the model by taking additional linear combinations. For example, with 4 genes one might use beta(1)*ALOX5+beta(2)*5100A6 along one axis and beta(3)*gene3+beta(4)*gene4 along the other, or beta(1)*ALOX5+ beta(2)*S100A6+beta(3)*gene3 along one axis and gene4 along the other axis. When producing such plots with 3 or more genes, genes with parameter estimates having the same sign were chosen for combination.

### Using R²Statistics to Rank Models

The R² in traditional OLS (ordinary least squares) linear regression of a continuous dependent variable can be interpreted in several different ways, such as 1) proportion of variance accounted for, 2) the squared correlation between the observed and predicted values, and 3) a transformation of the F-statistic. When the dependent variable is not continuous but categorical (in our models the dependent variable is dichotomous—membership in the disease A group or reference group (e.g., disease B)), this standard R² defined in terms of variance (see definition 1 above) is only one of several possible measures. The term ‘pseudo R²’ has been coined for the generalization of the standard variance-based R² for use with categorical dependent variables, as well as other settings where the usual assumptions that justify OLS do not apply.

The general definition of the (pseudo) R² for an estimated model is the reduction of errors compared to the errors of a baseline model. For the purpose of the present invention, the estimated model is a logistic regression model for predicting group membership based on 1 or more continuous predictors (ΔC_{T} measurements of different genes). The baseline model is the regression model that contains no predictors; that is, a model where the regression coefficients are restricted to 0. More precisely, the pseudo R² is defined as:

R²=[Error(baseline)−Error(model)]/Error(baseline)

Regardless how error is defined, if prediction is perfect, Error(model)=0 which yields R²=1. Similarly, if all of the regression coefficients do in fact turn out to equal 0, the model is equivalent to the baseline, and thus R²=0. In general, this pseudo R² falls somewhere between 0 and 1.

When Error is defined in terms of variance, the pseudo R² becomes the standard R². When the dependent variable is dichotomous group membership, scores of 1 and 0, −1 and +1, or any other 2 numbers for the 2 categories yields the same value for R². For example, if the dichotomous dependent variable takes on the scores of 1 and 0, the variance is defined as P*(1−P) where P is the probability of being in 1 group and 1−P the probability of being in the other.

A common alternative in the case of a dichotomous dependent variable, is to define error in terms of entropy. In this situation, entropy can be defined as P*ln(P)*(1−P)*ln(1−P) (for further discussion of the variance and the entropy based R², see Magidson, Jay, “Qualitative Variance, Entropy and Correlation Ratios for Nominal Dependent Variables,” Social Science Research 10 (June), pp. 177-194).

The R² statistic was used in the enumeration methods described herein to identify the “best” gene-model. R² can be calculated in different ways depending upon how the error variation and total observed variation are defined. For example, four different R² measures output by Latent GOLD are based on:

a) Standard variance and mean squared error (MSE)
b) Entropy and minus mean log-likelihood (-MLL)
c) Absolute variation and mean absolute error (MAE)
d) Prediction errors and the proportion of errors under modal assignment (PPE)

Each of these 4 measures equal 0 when the predictors provide zero discrimination between the groups, and equal 1 if the model is able to classify each subject into their actual group with 0 error. For each measure, Latent GOLD defines the total variation as the error of the baseline (intercept-only) model which restricts the effects of all predictors to 0. Then for each, R² is defined as the proportional reduction of errors in the estimated model compared to the baseline model. For the 2-gene cancer example used to illustrate the enumeration methodology described herein, the baseline model classifies all cases as being in the diseased group A since this group has a larger sample size, resulting in 50 misclassifications (all 50 reference subjects are misclassified) for a prediction error of 50/107=0.467. In contrast, there are only 10 prediction errors (=10/107=0.093) based on the 2-gene model using the modal assignment rule, thus yielding a prediction error R² of 1−0.093/0.467=0.8. As shown in Exhibit 1, 4 reference (e.g., Cancer B) and 6 cancer A subjects would be misclassified using the modal assignment rule. Note that the modal rule utilizes P₀=0.5 as the cutoff. If P₀=0.4 were used instead, there would be only 8 misclassified subjects.

In the sample discrimination plot shown in FIG. 1, the 2 genes in the model are ALOX5 and S100A6 and only 8 subjects are misclassified (4 blue circles corresponding to reference subjects fall to the right and below the line, while 4 red Xs corresponding to misclassified cancer A subjects lie above the line).

To reduce the likelihood of obtaining models that capitalize on chance variations in the observed samples the models may be limited to contain only M genes as predictors in the model. (Although a model may meet the significance criteria, it may overfit data and thus would not be expected to validate when applied to a new sample of subjects.) For example, for M=2, all models would be estimated which contain:

$$\begin{matrix}A . 1 \text{-} gene -- G such models \\ B . 2 \text{-} gene models -- ( \begin{matrix}G \\ 2\end{matrix} ) = G * ( G - 1 ) / 2 such models \\ C . 3 \text{-} gene models -- ( G 3 ) = G * ( G - 1 ) * ( G - 2 ) / 6\end{matrix}$$

TABLE B
ΔC_{T} Values and Model Predicted
Probability of Cancer for Each Subject

| ALOX5 | S100A6 | P | Group |
|---|---|---|---|
| 13.92 | 16.13 | 1.0000 | Cancer |
| 13.90 | 15.77 | 1.0000 | Cancer |
| 13.75 | 15.17 | 1.0000 | Cancer |
| 13.62 | 14.51 | 1.0000 | Cancer |
| 15.33 | 17.16 | 1.0000 | Cancer |
| 13.86 | 14.61 | 1.0000 | Cancer |
| 14.14 | 15.09 | 1.0000 | Cancer |
| 13.49 | 13.60 | 0.9999 | Cancer |
| 15.24 | 16.61 | 0.9999 | Cancer |
| 14.03 | 14.45 | 0.9999 | Cancer |
| 14.98 | 16.05 | 0.9999 | Cancer |
| 13.95 | 14.25 | 0.9999 | Cancer |
| 14.09 | 14.13 | 0.9998 | Cancer |
| 15.01 | 15.69 | 0.9997 | Cancer |
| 14.13 | 14.15 | 0.9997 | Cancer |
| 14.37 | 14.43 | 0.9996 | Cancer |
| 14.14 | 13.88 | 0.9994 | Cancer |
| 14.33 | 14.17 | 0.9993 | Cancer |
| 14.97 | 15.06 | 0.9988 | Cancer |
| 14.59 | 14.30 | 0.9984 | Cancer |
| 14.45 | 13.93 | 0.9978 | Cancer |
| 14.40 | 13.77 | 0.9972 | Cancer |
| 14.72 | 14.31 | 0.9971 | Cancer |
| 14.81 | 14.38 | 0.9963 | Cancer |
| 14.54 | 13.91 | 0.9963 | Cancer |
| 14.88 | 14.48 | 0.9962 | Cancer |
| 14.85 | 14.42 | 0.9959 | Cancer |
| 15.40 | 15.30 | 0.9951 | Cancer |
| 15.58 | 15.60 | 0.9951 | Cancer |
| 14.82 | 14.28 | 0.9950 | Cancer |
| 14.78 | 14.06 | 0.9924 | Cancer |
| 14.68 | 13.88 | 0.9922 | Cancer |
| 14.54 | 13.64 | 0.9922 | Cancer |
| 15.86 | 15.91 | 0.9920 | Cancer |
| 15.71 | 15.60 | 0.9908 | Cancer |
| 16.24 | 16.36 | 0.9858 | Cancer |
| 16.09 | 15.94 | 0.9774 | Cancer |
| 15.26 | 14.41 | 0.9705 | Cancer |
| 14.93 | 13.81 | 0.9693 | Cancer |
| 15.44 | 14.67 | 0.9670 | Cancer |
| 15.69 | 15.08 | 0.9663 | Cancer |
| 15.40 | 14.54 | 0.9615 | Cancer |
| 15.80 | 15.21 | 0.9586 | Cancer |
| 15.98 | 15.43 | 0.9485 | Cancer |
| 15.20 | 14.08 | 0.9461 | Normal |
| 15.03 | 13.62 | 0.9196 | Cancer |
| 15.20 | 13.91 | 0.9184 | Cancer |
| 15.04 | 13.54 | 0.8972 | Cancer |
| 15.30 | 13.92 | 0.8774 | Cancer |
| 15.80 | 14.68 | 0.8404 | Cancer |
| 15.61 | 14.23 | 0.7939 | Normal |
| 15.89 | 14.64 | 0.7577 | Normal |
| 15.44 | 13.66 | 0.6445 | Cancer |
| 16.52 | 15.38 | 0.5343 | Cancer |
| 15.54 | 13.67 | 0.5255 | Normal |
| 15.28 | 13.11 | 0.4537 | Cancer |
| 15.96 | 14.23 | 0.4207 | Cancer |
| 15.96 | 14.20 | 0.3928 | Normal |
| 16.25 | 14.69 | 0.3887 | Cancer |
| 16.04 | 14.32 | 0.3874 | Cancer |
| 16.26 | 14.71 | 0.3863 | Normal |
| 15.97 | 14.18 | 0.3710 | Cancer |
| 15.93 | 14.06 | 0.3407 | Normal |
| 16.23 | 14.41 | 0.2378 | Cancer |
| 16.02 | 13.91 | 0.1743 | Normal |
| 15.99 | 13.78 | 0.1501 | Normal |
| 16.74 | 15.05 | 0.1389 | Normal |
| 16.66 | 14.90 | 0.1349 | Normal |
| 16.91 | 15.20 | 0.0994 | Normal |
| 16.47 | 14.31 | 0.0721 | Normal |
| 16.63 | 14.57 | 0.0672 | Normal |
| 16.25 | 13.90 | 0.0663 | Normal |
| 16.82 | 14.84 | 0.0596 | Normal |
| 16.75 | 14.73 | 0.0587 | Normal |
| 16.69 | 14.54 | 0.0474 | Normal |
| 17.13 | 15.25 | 0.0416 | Normal |
| 16.87 | 14.72 | 0.0329 | Normal |
| 16.35 | 13.76 | 0.0285 | Normal |
| 16.41 | 13.83 | 0.0255 | Normal |
| 16.68 | 14.20 | 0.0205 | Normal |
| 16.58 | 13.97 | 0.0169 | Normal |
| 16.66 | 14.09 | 0.0167 | Normal |
| 16.92 | 14.49 | 0.0140 | Normal |
| 16.93 | 14.51 | 0.0139 | Normal |
| 17.27 | 15.04 | 0.0123 | Normal |
| 16.45 | 13.60 | 0.0116 | Normal |
| 17.52 | 15.44 | 0.0110 | Normal |
| 17.12 | 14.46 | 0.0051 | Normal |
| 17.13 | 14.46 | 0.0048 | Normal |
| 16.78 | 13.86 | 0.0047 | Normal |
| 17.10 | 14.36 | 0.0041 | Normal |
| 16.75 | 13.69 | 0.0034 | Normal |
| 17.27 | 14.49 | 0.0027 | Normal |
| 17.07 | 14.08 | 0.0022 | Normal |
| 17.16 | 14.08 | 0.0014 | Normal |
| 17.50 | 14.41 | 0.0007 | Normal |
| 17.50 | 14.18 | 0.0004 | Normal |
| 17.45 | 14.02 | 0.0003 | Normal |
| 17.53 | 13.90 | 0.0001 | Normal |
| 18.21 | 15.06 | 0.0001 | Normal |
| 17.99 | 14.63 | 0.0001 | Normal |
| 17.73 | 14.05 | 0.0001 | Normal |
| 17.97 | 14.40 | 0.0001 | Normal |
| 17.98 | 14.35 | 0.0001 | Normal |
| 18.47 | 15.16 | 0.0001 | Normal |
| 18.28 | 14.59 | 0.0000 | Normal |
| 18.37 | 14.71 | 0.0000 | Normal |

### Example 3

### Precision Profile™ for Inflammatory Response

Custom primers and probes were prepared for the targeted 72 genes shown in the Precision Profile™ for Inflammatory Response (shown in Table A), selected to be informative relative to biological state of inflammation and cancer. Gene expression profiles for the 72 inflammatory response genes were analyzed using the RNA samples obtained from the melanoma (N=26, all stages, active disease), lung cancer (N=49, all stages), colon cancer (N=18), prostate cancer (N=40, all stages), ovarian cancer (N=23, all stages), breast cancer (N=49, all stages), and cervical cancer (N=24, all stages) subjects, described in Example 1, to compare one type of cancer (Cancer A) to another type of cancer (Cancer B). The following 18 combinations of cancer versus cancer comparisons were analyzed to identify logistic regression gene-models based on the Precision Profile™ for Inflammatory Response (Table A) capable of distinguishing between subjects having one type of cancer (i.e., Cancer A) versus subjects having another type of cancer (i.e., Cancer B): breast cancer vs. melanoma; breast cancer vs. ovarian cancer; cervical cancer vs. breast cancer; cervical cancer vs. colon cancer; cervical cancer vs. melanoma; cervical cancer vs. ovarian cancer; colon cancer vs. melanoma; lung cancer vs. breast cancer; lung cancer vs. cervical cancer; lung cancer vs. colon cancer; lung cancer vs. melanoma; lung cancer vs. ovarian cancer; lung cancer vs. prostate cancer; ovarian cancer vs. colon cancer; ovarian cancer vs. melanoma; prostate cancer vs. colon cancer; prostate cancer vs. melanoma; and breast cancer vs. colon cancer.

Logistic regression models yielding the best discrimination between subjects diagnosed with one type of cancer (Cancer A) versus another type of cancer (Cancer B) were generated using the enumeration and classification methodology described in Example 2. A listing of all 1 and 2-gene logistic regression models capable of distinguishing between subjects diagnosed with Cancer A and subjects diagnosed with Cancer B with at least 75% accuracy are shown in Tables A1a-A18a, read from left to right.

Table A1a lists all 1 and 2-gene models capable of distinguishing between subjects with breast cancer and melanoma (active disease, all stages) with at least 75% accuracy. Table A2a lists all 1 and 2-gene models capable of distinguishing between subjects with breast cancer and ovarian cancer with at least 75% accuracy. Table A3a lists all 1 and 2-gene models capable of distinguishing between subjects with cervical cancer and breast cancer with at least 75% accuracy. Table A4a lists all 1 and 2-gene models capable of distinguishing between subjects with cervical cancer and colon cancer with at least 75% accuracy. Table A5a lists all 1 and 2-gene models capable of distinguishing between subjects with cervical cancer and melanoma (active disease, all stages) with at least 75% accuracy. Table A6a lists all 1 and 2-gene models capable of distinguishing between subjects with cervical cancer and ovarian cancer with at least 75% accuracy. Table A1a lists all 1 and 2-gene models capable of distinguishing between subjects with colon cancer and melanoma (active disease, all stages) with at least 75% accuracy. Table A8a lists all 1 and 2-gene models capable of distinguishing between subjects with lung cancer and breast cancer with at least 75% accuracy. Table A9a lists all 1 and 2-gene models capable of distinguishing between subjects with lung cancer and cervical cancer with at least 75% accuracy. Table A10a lists all 1 and 2-gene models capable of distinguishing between subjects with lung cancer and colon cancer with at least 75% accuracy. Table A11a lists all 1 and 2-gene models capable of distinguishing between subjects with lung cancer and melanoma (active disease, all stages) with at least 75% accuracy. Table A12a lists all 1 and 2-gene models capable of distinguishing between subjects with lung cancer and ovarian cancer with at least 75% accuracy. Table A13a lists all 1 and 2-gene models capable of distinguishing between subjects with lung cancer and prostate cancer with at least 75% accuracy. Table A14a lists all 1 and 2-gene models capable of distinguishing between subjects with ovarian cancer and colon cancer with at least 75% accuracy. Table A15a lists all 1 and 2-gene models capable of distinguishing between subjects with ovarian cancer and melanoma (active disease, all stages) with at least 75% accuracy. Table A16a lists all 1 and 2-gene models capable of distinguishing between subjects with prostate cancer and colon cancer with at least 75% accuracy. Table A17a lists all 1 and 2-gene models capable of distinguishing between subjects with prostate cancer and melanoma (active disease, all stages) with at least 75% accuracy. Table A18a lists all 1 and 2-gene models capable of distinguishing between subjects with breast cancer and colon cancer with at least 75% accuracy.

As shown in Tables A1a-A18a, the 1 and 2-gene models are identified in the first two columns on the left side of each table, ranked by their entropy R² value (shown in column 3, ranked from high to low). The number of subjects correctly classified or misclassified by each 1 or 2-gene model for each patient group (i.e., Cancer A vs. Cancer B) is shown in columns 4-7. The percent Cancer A subjects and Cancer B subjects correctly classified by the corresponding gene model is shown in columns 8 and 9. The incremental p-value for each first and second gene in the 1 or 2-gene model is shown in columns 10-11 (note p-values smaller than 1×10⁻¹⁷ are reported as ‘0’). The total number of RNA samples analyzed in each patient group (i.e., Cancer A vs. Cancer B) after exclusion of missing values, is shown in columns 12-13. The values missing from the total sample number for Cancer A and/or Cancer B subjects shown in columns 12-13 correspond to instances in which values were excluded from the logistic regression analysis due to reagent limitations and/or instances where replicates did not meet quality metrics.

The “best” logistic regression model (defined as the model with the highest entropy R² value, as described in Example 2) based on the 72 genes included in the Precision Profile™ for Inflammatory Response for each of the 18 combinations of cancer vs. cancer comparisons is shown in the first row of Tables A1a-A18a, respectively. For example, the first row of Table A1a lists a 2-gene model, ALOX5 and PLAUR, capable of classifying breast cancer subjects with 100% accuracy, and melanoma (active disease, all stages) subjects with 100% accuracy. All 26 melanoma and all 49 breast cancer RNA samples were analyzed for this 2-gene model, no values were excluded. As shown in Table A1a, this 2-gene model correctly classifies all 26 of the melanoma subjects as being in the melanoma patient population, and correctly classifies all 49 breast cancer subjects as being in the breast cancer patient population. The p-value for the 1^{st} gene, ALOX5, is 1.3E-08, the incremental p-value for the second gene, PLAUR is smaller than 1×10⁻¹⁷ (reported as 0).

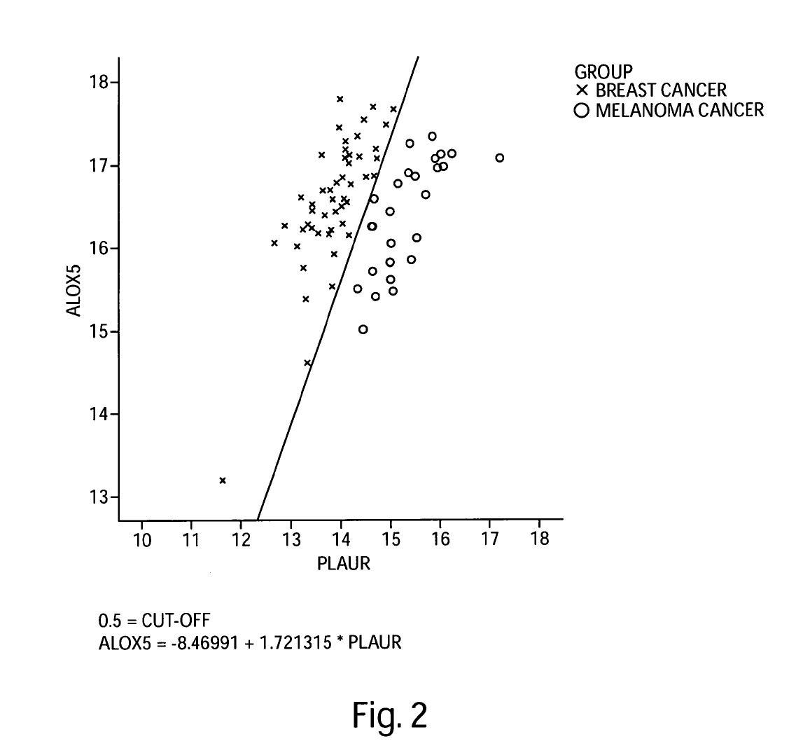

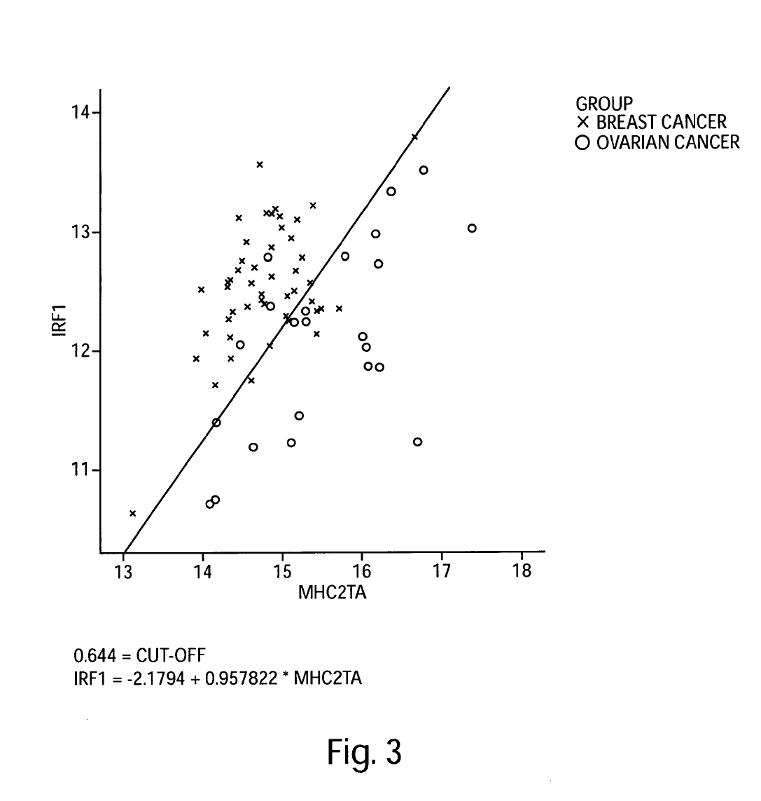

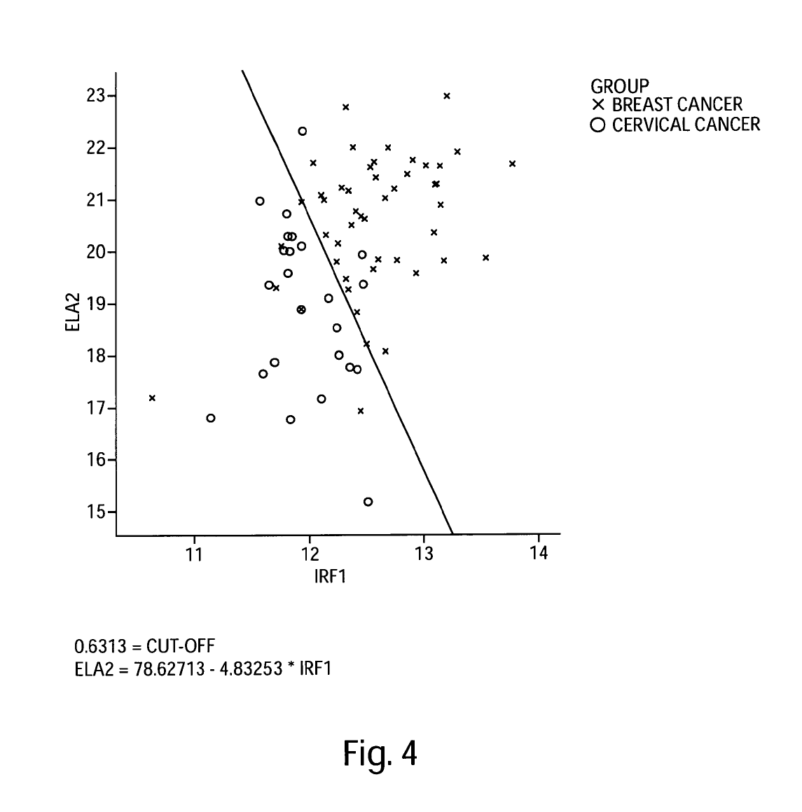

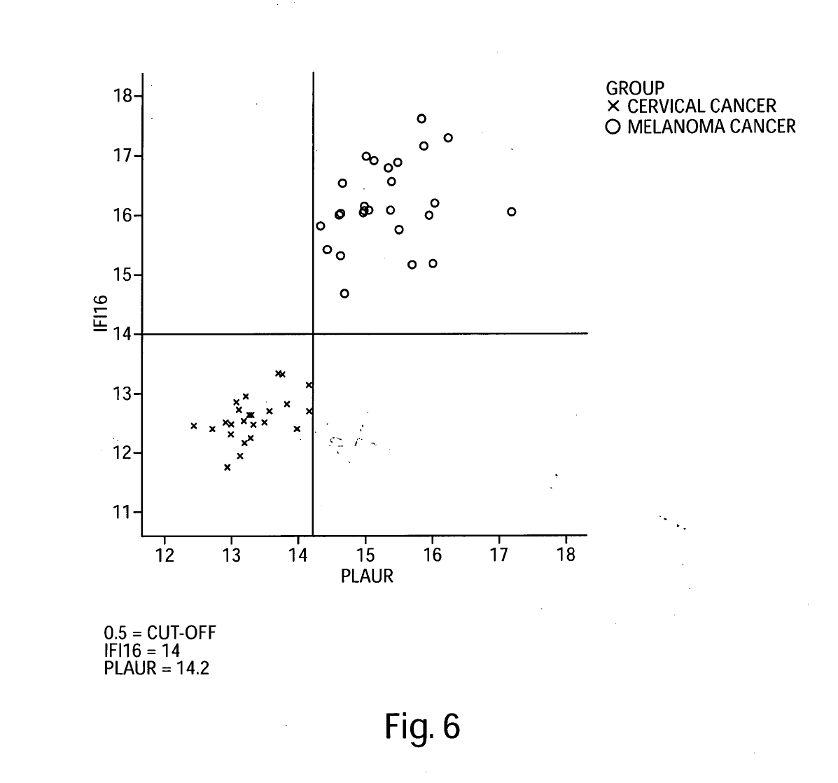

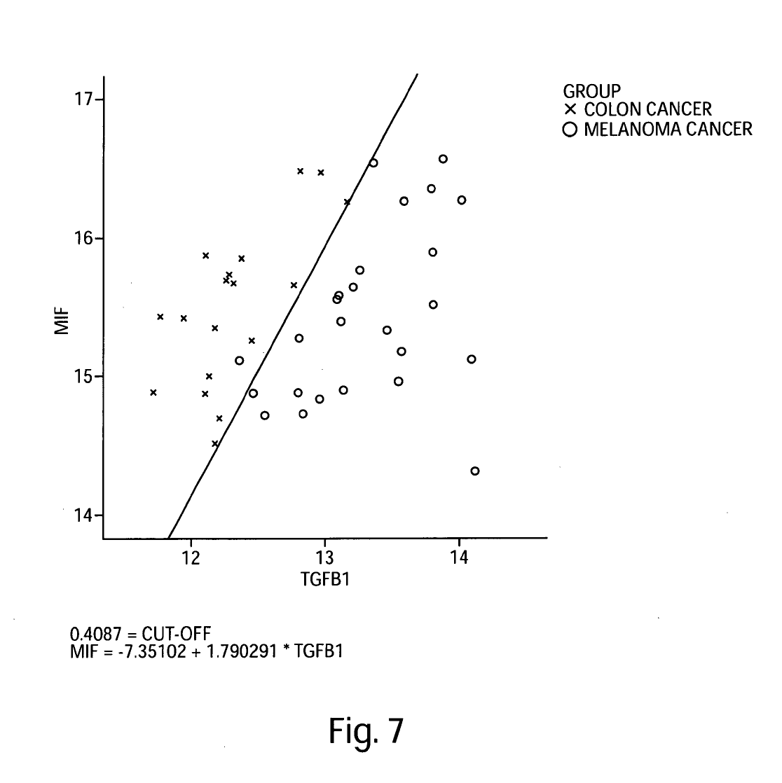

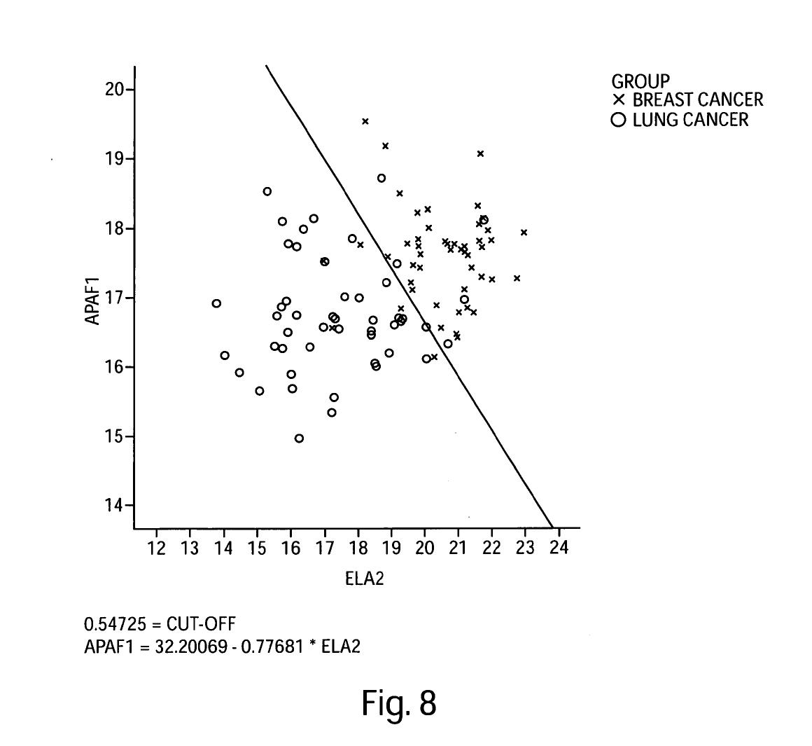

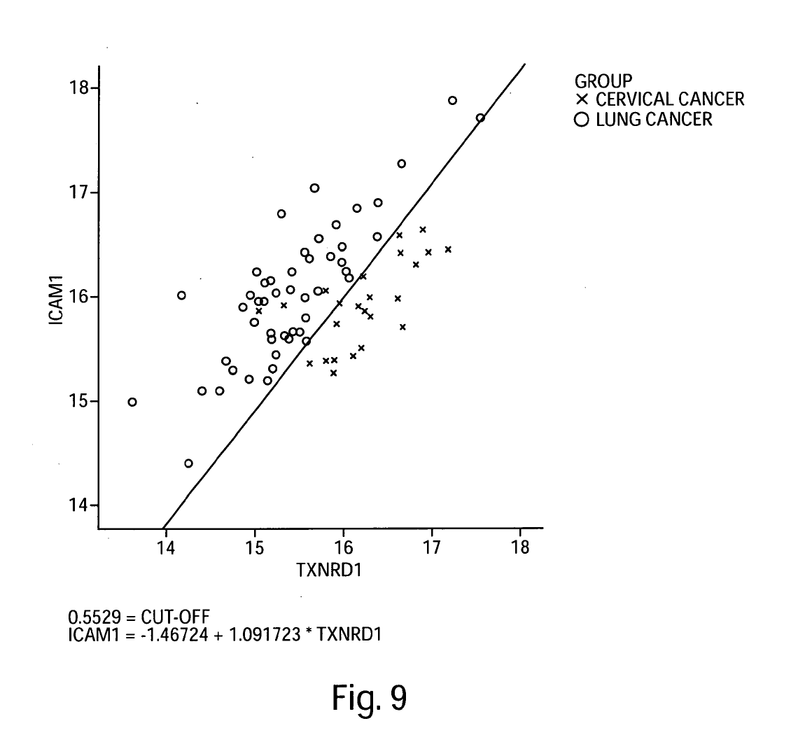

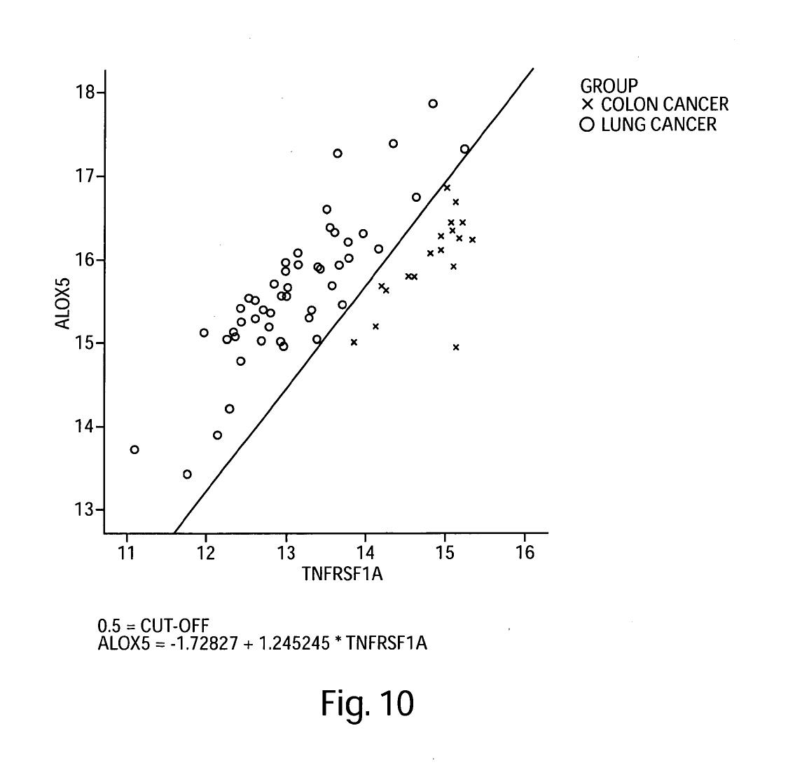

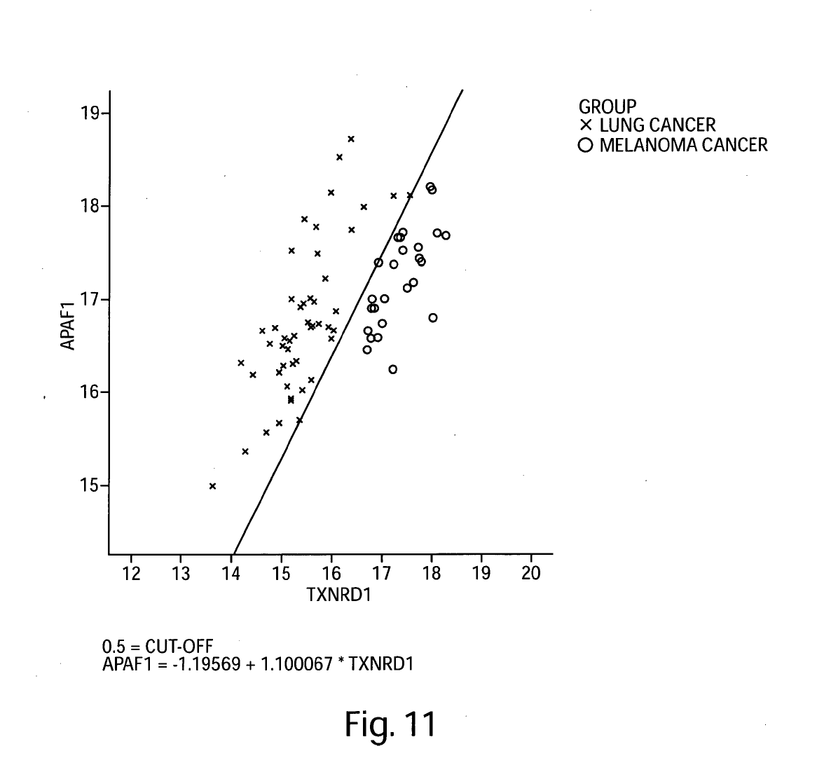

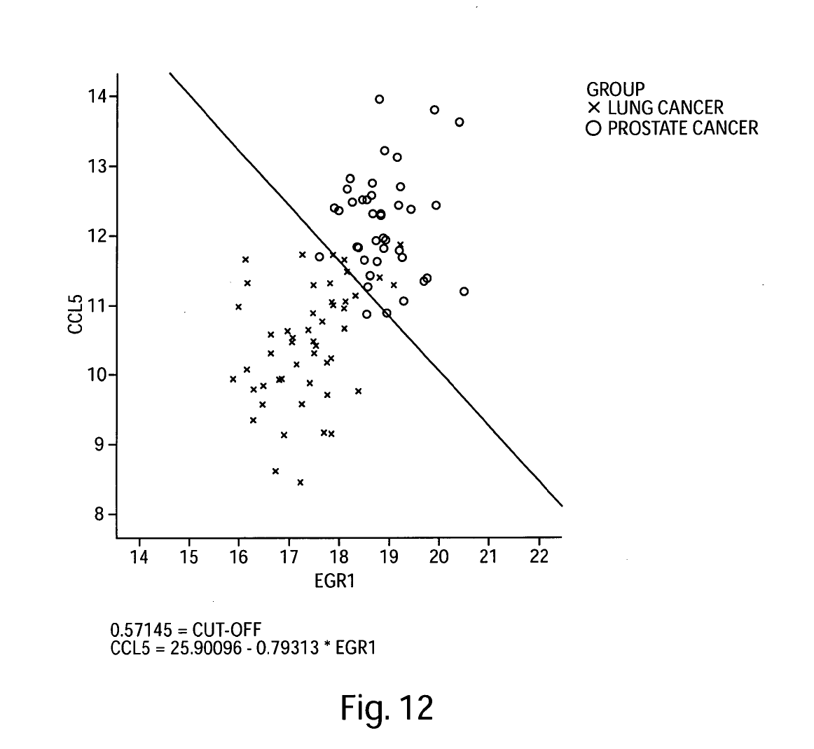

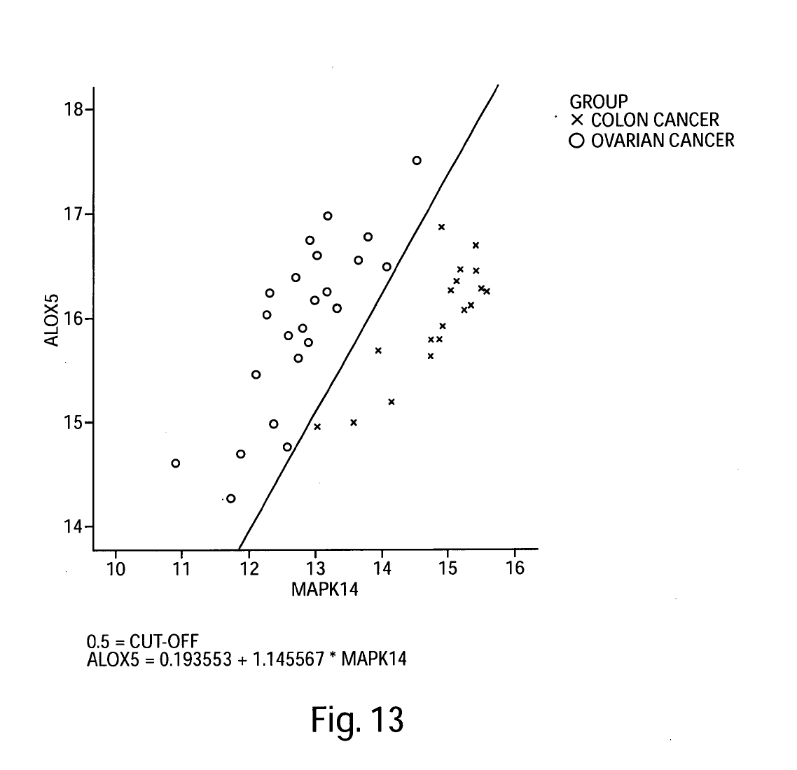

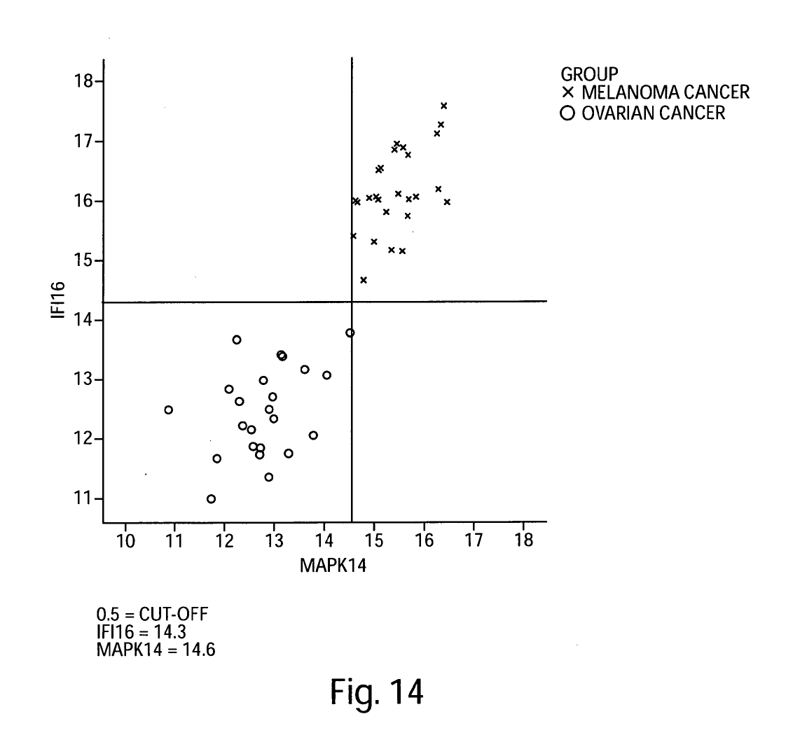

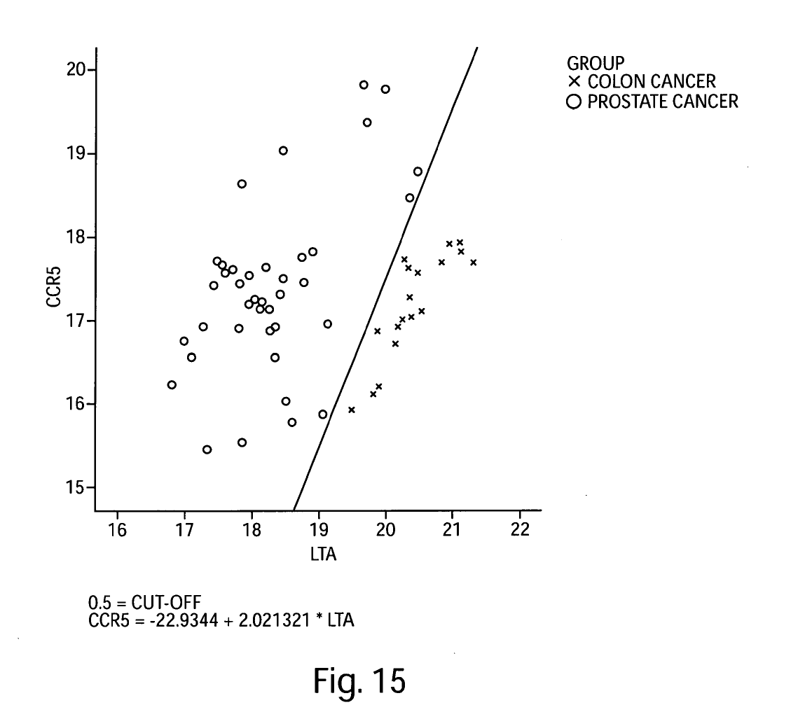

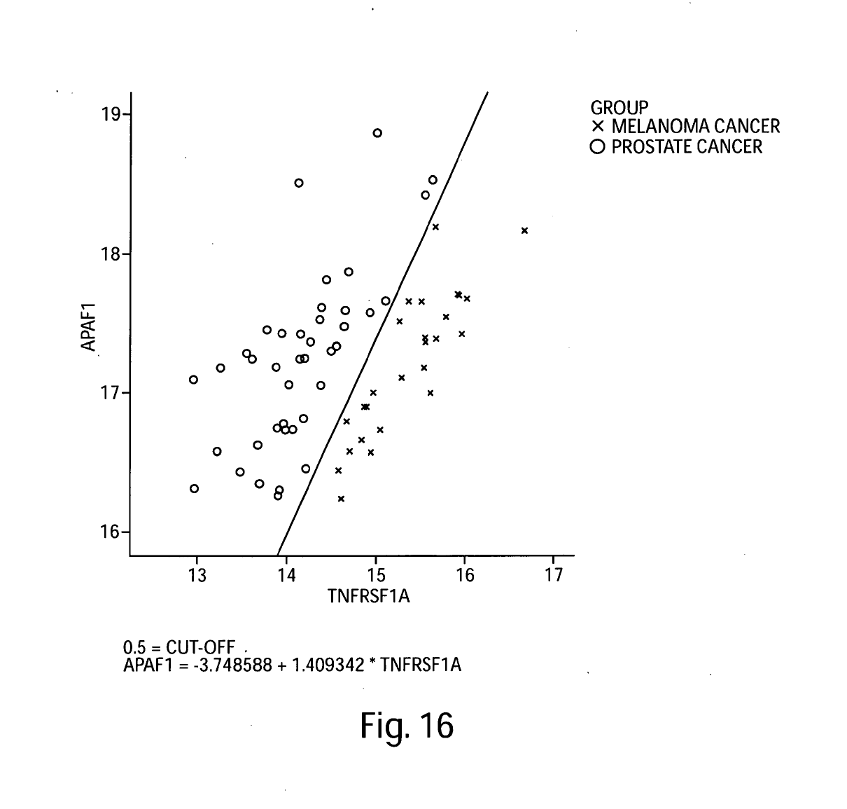

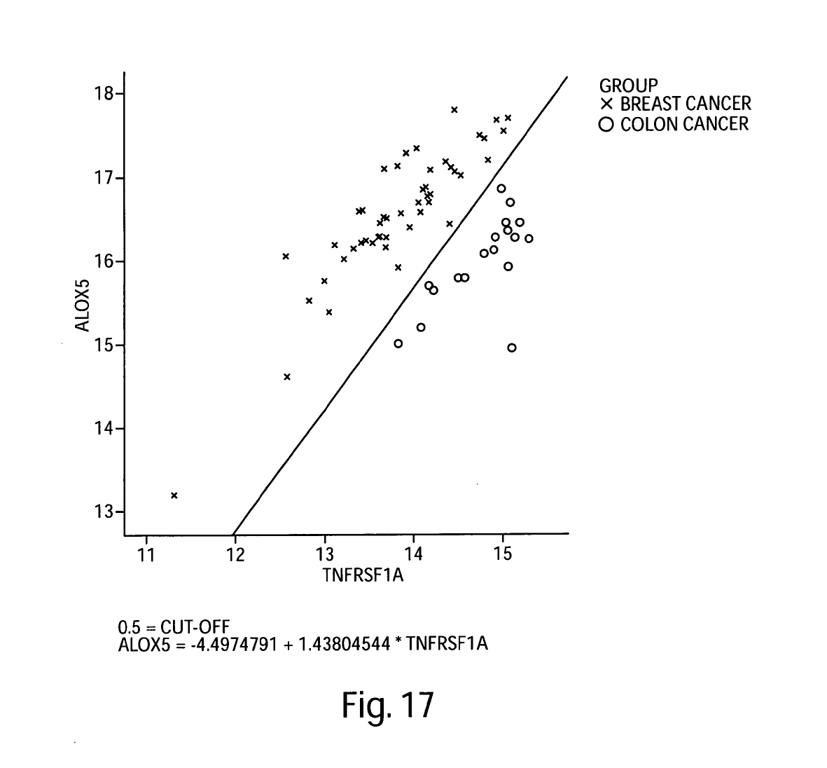

FIGS. 2-17 are discrimination plots based on the Precision Profile™ for Inflammatory Response, capable of distinguishing between Cancer A vs. Cancer B with at least 75% accuracy, for some of the “best” 2-gene models listed in Tables A1a-A18a, as described above in the ‘Brief Description of the Drawings’. For example, FIG. 2 is a graphical representation of the “best” logistic regression model, ALOX5, and PLAUR (identified in Table A1a), based on the Precision Profile™ for Inflammation (Table A), capable of distinguishing between subjects afflicted with breast cancer and subjects afflicted with melanoma (active disease, all stages). The discrimination line appended to FIG. 2 illustrates how well the 2-gene model discriminates between the 2 groups. Values to the left of the line represent subjects predicted to be in the breast cancer population. Values to the right of the line represent subjects predicted to be in the melanoma population (active disease, all stages). As shown in FIG. 2, zero breast cancer subjects (X's) and zero melanoma subjects (circles) are classified in the wrong patient population.

The cut-off value used to generate the discrimination line, and the line equation are shown below FIGS. 2-17, respectively. The slope and intercept of the discrimination lines were determined as previously described in Example 2. For example, the equation for the discrimination line shown in FIG. 2 is:

ALOX5=−8.46991+1.721315*PLAUR

The intercept (alpha) and slope (beta) of the discrimination line was computed as follows: A cutoff of 0.5 was used to compute alpha (equals 0 logit units).

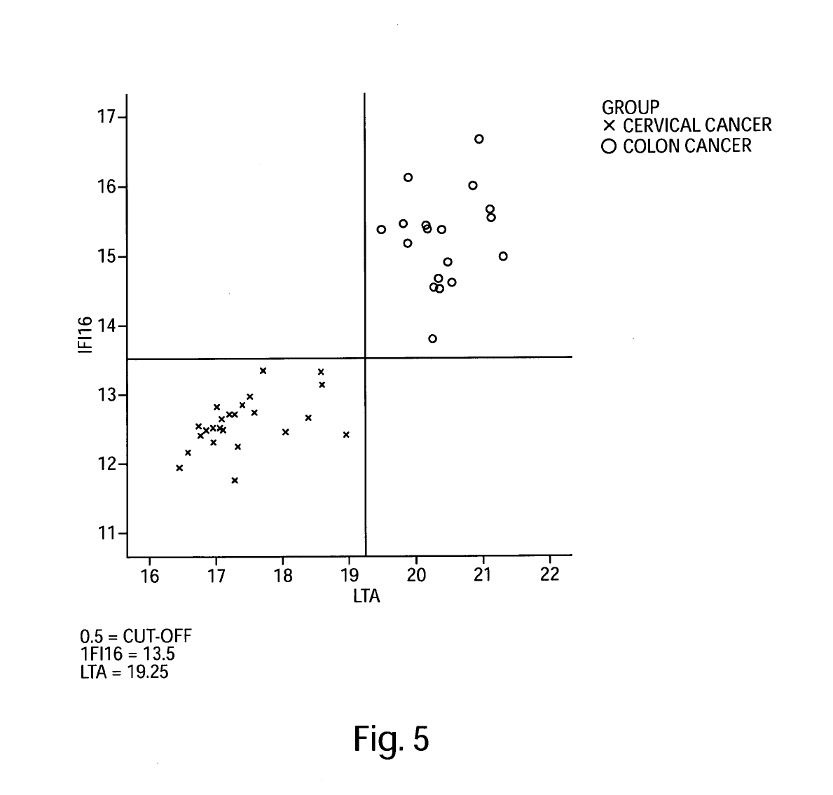

The intercept C₀=−8.46991 was computed by taking the difference between the intercepts for the 2 groups [434.819−(−434.819)=869.638] and subtracting the log-odds of the cutoff probability (0). This quantity was then multiplied by −1/X where X is the coefficient for ALOX5 (102.6738). Note that in some instances, as shown in FIGS. 5, 6, and 14, where the X and Y axis are each based on a 1-gene model, each of which provides 100% classification for each of the two groups when taken separately, both a horizontal and vertical discrimination line are appended to the graphs.

A ranking of the top 68 inflammatory response genes for which gene expression profiles were obtained, from most to least significant, is shown in Tables A1b-A18b. Tables A1b-A18b summarizes the results of significance tests (p-values) for the difference in the mean expression levels for Cancer A subjects and Cancer B subjects, for each of the 18 cancer vs. cancer comparisons, respectively.

In some instances, also provided are the expression values (ΔC_{T}) for each of the Cancer A and Cancer B subjects used to analyze the “best” gene model (after exclusion of missing values) and their predicted probability of having Cancer A vs. Cancer B, as shown in Tables A1c-A5c, A7c-A11c, and A13c-A18c. For example, as shown in Table A1c, the predicted probability of a subject having breast cancer versus melanoma (active disease, all stages), based on the 2-gene model ALOX5 and PLAUR (identified in Table A1a) is based on a scale of 0 to 1, “0” indicating the subject has melanoma (active disease, all stages) “1” indicating the subject has breast cancer. This predicted probability can be used to create an index based on the 2-gene model ALOX5 and PLAUR that can be used as a tool by a practitioner (e.g., primary care physician, oncologist, etc.) for diagnosis of breast cancer versus melanoma (active disease, all stages), and to ascertain the necessity of future screening or treatment options.

### Example 4

### Human Cancer General Precision Profile™

Custom primers and probes were prepared for the targeted 91 genes shown in the Human Cancer General Precision Profile™ (shown in Table B), selected to be informative relative to the biological condition of human cancer, including but not limited to ovarian, breast, cervical, prostate, lung, colon, and skin cancer. Gene expression profiles for these 91 genes were analyzed using the RNA samples obtained from the melanoma (N=49, stages 2-4, active disease), lung cancer (N=49, all stages), colon cancer (N=23), prostate cancer (N=57, all stages), ovarian cancer (N=21, all stages), breast cancer (N=49, all stages), and cervical cancer (N=24, all stages) subjects, described in Example 1, to compare one type of cancer (Cancer A) to another type of cancer (Cancer B). The following 18 combinations of cancer versus cancer comparisons were analyzed to identify logistic regression gene-models based on the Human Cancer General Precision Profile™ (Table B) capable of distinguishing between subjects having one type of cancer (i.e., Cancer A) versus subjects having another type of cancer (i.e., Cancer B): breast cancer vs. melanoma; breast cancer vs. ovarian cancer; cervical cancer vs. breast cancer; cervical cancer vs. colon cancer; cervical cancer vs. melanoma; cervical cancer vs. ovarian cancer; colon cancer vs. melanoma; lung cancer vs. breast cancer; lung cancer vs. cervical cancer; lung cancer vs. colon cancer; lung cancer vs. melanoma; lung cancer vs. ovarian cancer; lung cancer vs. prostate cancer; ovarian cancer vs. colon cancer; ovarian cancer vs. melanoma; prostate cancer vs. colon cancer; prostate cancer vs. melanoma; and breast cancer vs. colon cancer.

Logistic regression models yielding the best discrimination between subjects diagnosed with one type of cancer (Cancer A) versus another type of cancer (Cancer B) were generated using the enumeration and classification methodology described in Example 2. A listing of all 1 and 2-gene logistic regression models capable of distinguishing between subjects diagnosed with Cancer A and subjects diagnosed with Cancer B with at least 75% accuracy are shown in Tables B1a-B18a, read from left to right.

Table B1a lists all 1 and 2-gene models capable of distinguishing between subjects with breast cancer and melanoma (active disease, stages 2-4) with at least 75% accuracy. Table B2a lists all 1 and 2-gene models capable of distinguishing between subjects with breast cancer and ovarian cancer with at least 75% accuracy. Table B3a lists all 1 and 2-gene models capable of distinguishing between subjects with cervical cancer and breast cancer with at least 75% accuracy. Table B4a lists all 1 and 2-gene models capable of distinguishing between subjects with cervical cancer and colon cancer with at least 75% accuracy. Table B5a lists all 1 and 2-gene models capable of distinguishing between subjects with cervical cancer and melanoma (active disease, stages 2-4) with at least 75% accuracy. Table B6a lists all 1 and 2-gene models capable of distinguishing between subjects with cervical cancer and ovarian cancer with at least 75% accuracy. Table B7a lists all 1 and 2-gene models capable of distinguishing between subjects with colon cancer and melanoma (active disease, stages 2-4) with at least 75% accuracy. Table B8a lists all 1 and 2-gene models capable of distinguishing between subjects with lung cancer and breast cancer with at least 75% accuracy. Table B9a lists all 1 and 2-gene models capable of distinguishing between subjects with lung cancer and cervical cancer with at least 75% accuracy. Table B10a lists all 1 and 2-gene models capable of distinguishing between subjects with lung cancer and colon cancer with at least 75% accuracy. Table B11a lists all 1 and 2-gene models capable of distinguishing between subjects with lung cancer and melanoma (active disease, stages 2-4) with at least 75% accuracy. Table B12a lists all 2-gene models capable of distinguishing between subjects with lung cancer and ovarian cancer with at least 75% accuracy. Table B13a lists all 1 and 2-gene models capable of distinguishing between subjects with lung cancer and prostate cancer with at least 75% accuracy. Table B14a lists all 1 and 2-gene models capable of distinguishing between subjects with ovarian cancer and colon cancer with at least 75% accuracy. Table B15a lists all 1 and 2-gene models capable of distinguishing between subjects with ovarian cancer and melanoma (active disease, stages 2-4) with at least 75% accuracy. Table B16a lists all 1 and 2-gene models capable of distinguishing between subjects with prostate cancer and colon cancer with at least 75% accuracy. Table B17a lists all 1 and 2-gene models capable of distinguishing between subjects with prostate cancer and melanoma (active disease, stages 2-4) with at least 75% accuracy. Table B18a lists all 2-gene models capable of distinguishing between subjects with breast cancer and colon cancer with at least 75% accuracy.

As shown in Tables B1a-B18a, the 1 and 2-gene models are identified in the first two columns on the left side of each table, ranked by their entropy R² value (shown in column 3, ranked from high to low). The number of subjects correctly classified or misclassified by each 1 or 2-gene model for each patient group (i.e., Cancer A vs. Cancer B) is shown in columns 4-7. The percent Cancer A subjects and Cancer B subjects correctly classified by the corresponding gene model is shown in columns 8 and 9. The incremental p-value for each first and second gene in the 1 or 2-gene model is shown in columns 10-11 (note p-values smaller than 1×10⁻¹⁷ are reported as ‘0’). The total number of RNA samples analyzed in each patient group (i.e., Cancer A vs. Cancer B) after exclusion of missing values, is shown in columns 12-13. The values missing from the total sample number for Cancer A and/or Cancer B subjects shown in columns 12-13 correspond to instances in which values were excluded from the logistic regression analysis due to reagent limitations and/or instances where replicates did not meet quality metrics.

The “best” logistic regression model (defined as the model with the highest entropy R² value, as described in Example 2) based on the 91 genes included in the Human Cancer General Precision Profile™ for each of the 18 combinations of cancer vs. cancer comparisons is shown in the first row of Tables B1a-B18a, respectively. For example, the first row of Table B1a lists a 2-gene model, RAF1 and TGFB1, capable of classifying melanoma subjects (active disease, stages 2-4) with 93.9% accuracy, and breast cancer subjects with 91.8% accuracy. All 49 melanoma and all 49 breast cancer RNA samples were analyzed for this 2-gene model, no values were excluded. As shown in Table B1a, this 2-gene model correctly classifies all 46 of the melanoma subjects as being in the melanoma patient population, and misclassifies 3 of the melanoma subjects as being in the breast cancer population. This 2-gene model correctly classifies 45 of the breast cancer subjects as being in the breast cancer patient population and misclassifies 4 of the breast cancer subjects as being in the melanoma patient population. The p-value for the 1^{st} gene, RAF1 is 3.9E-08, the incremental p-value for the second gene, TGFB1 is smaller than 1×10⁻¹⁷ (reported as 0).

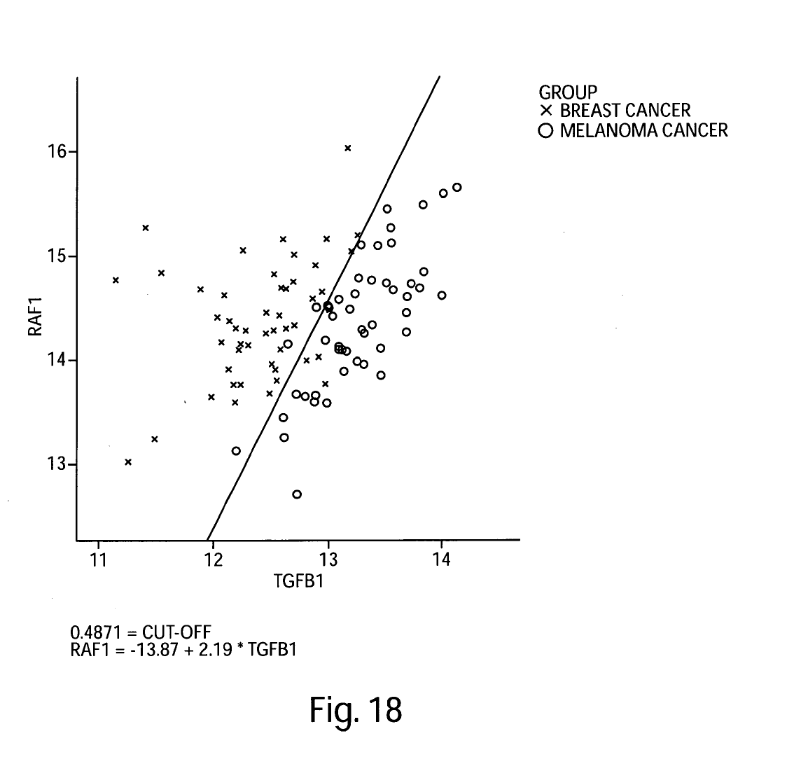

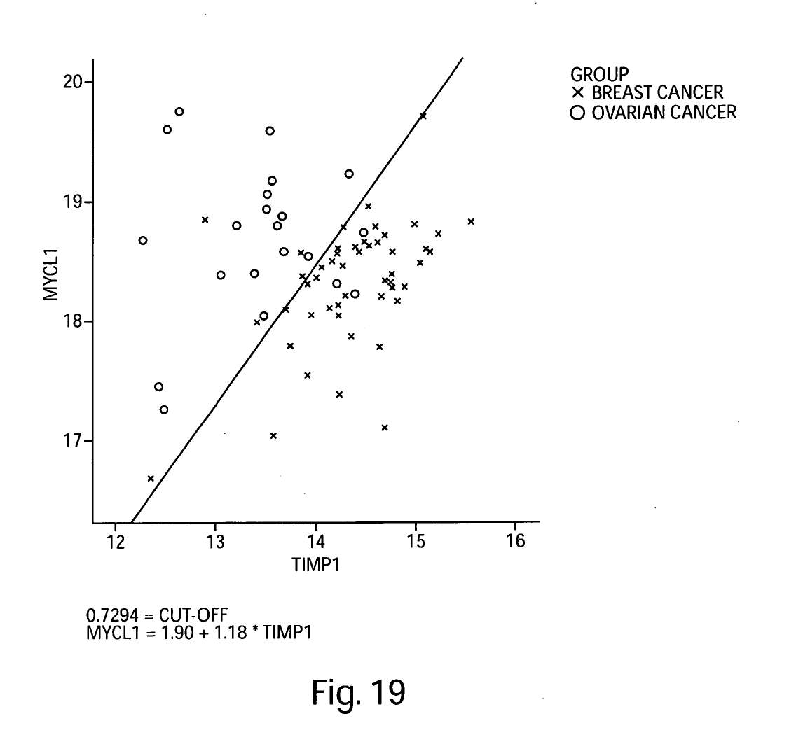

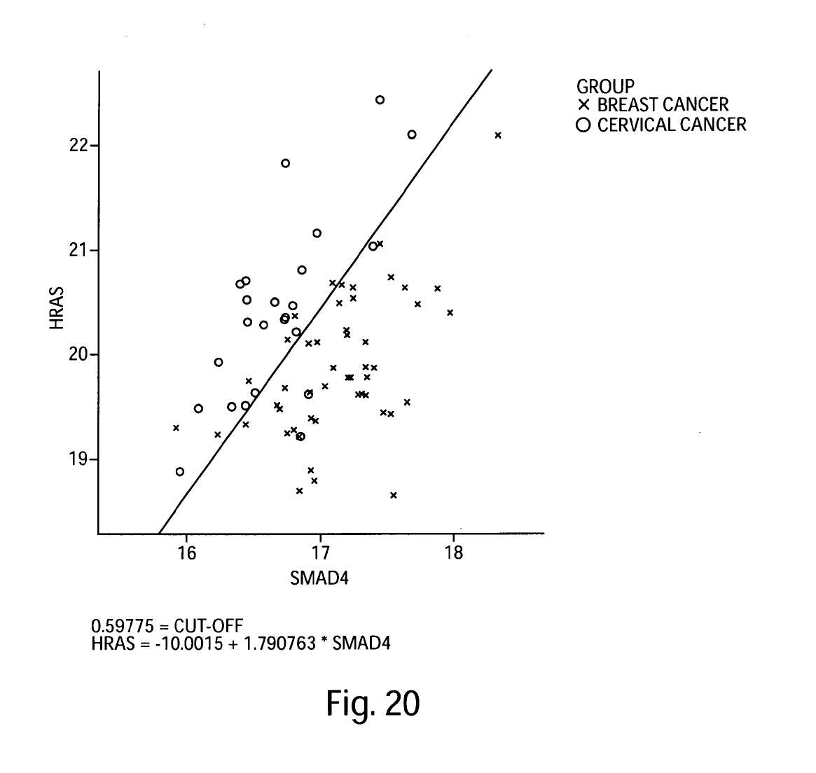

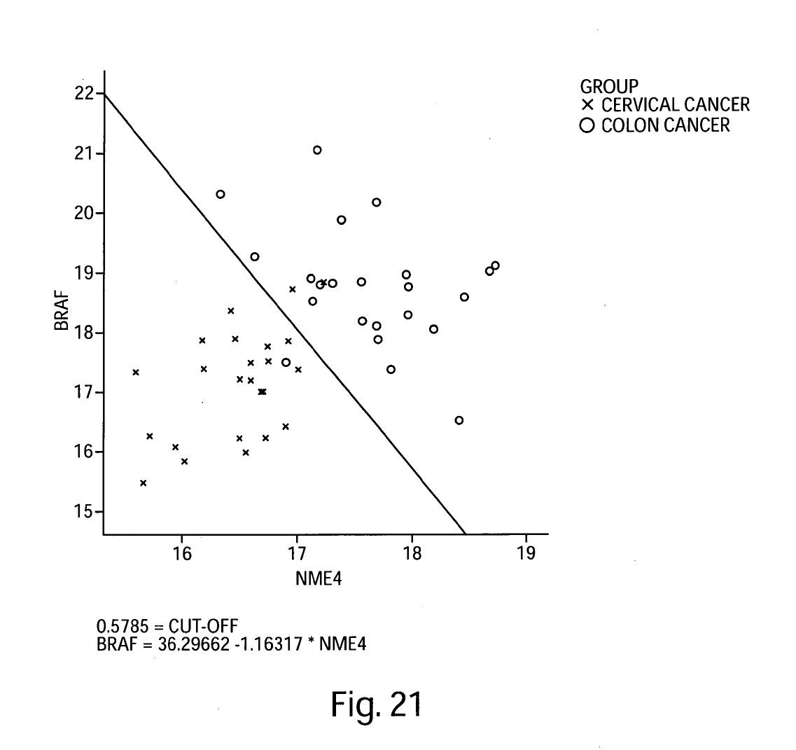

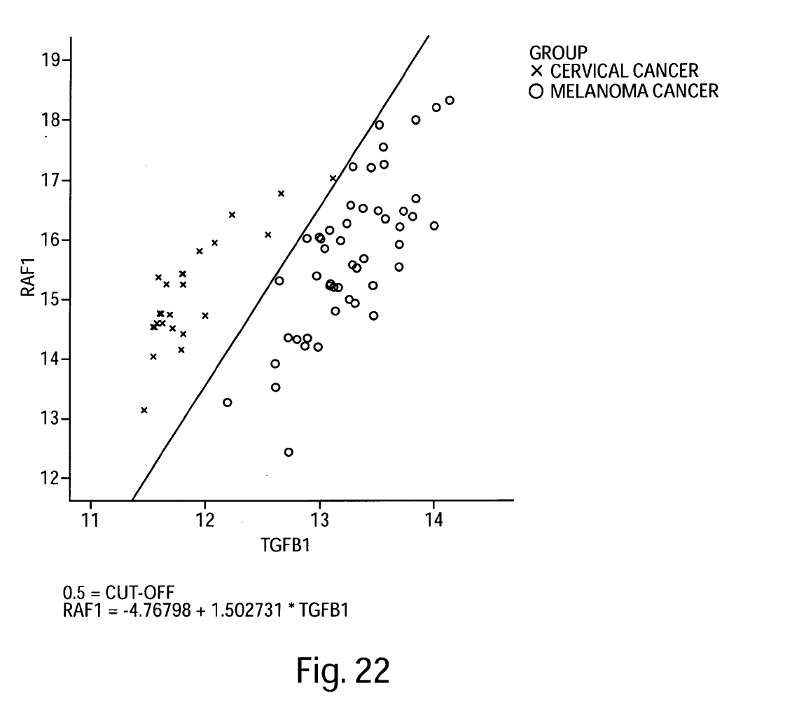

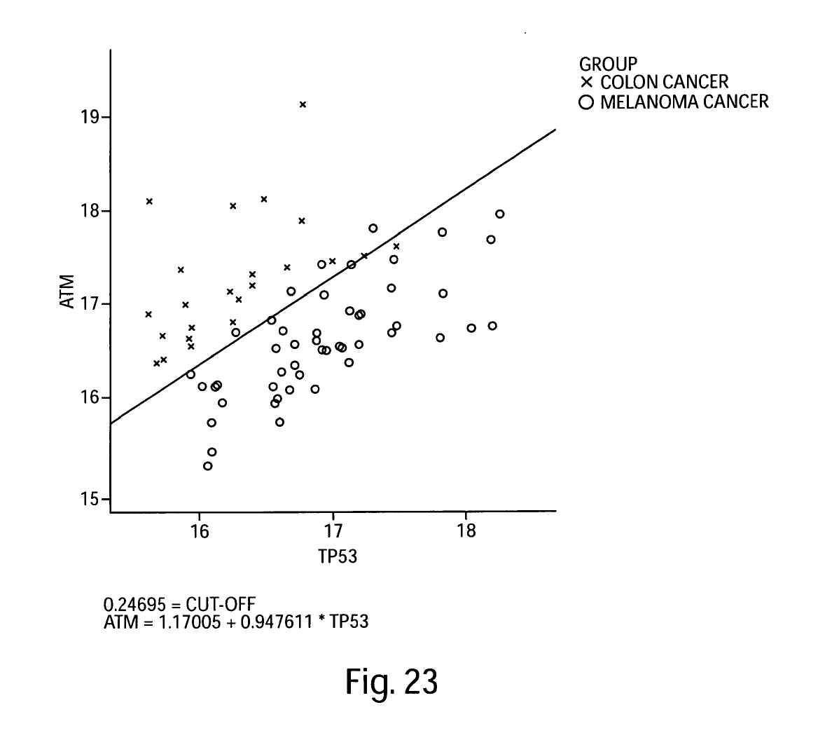

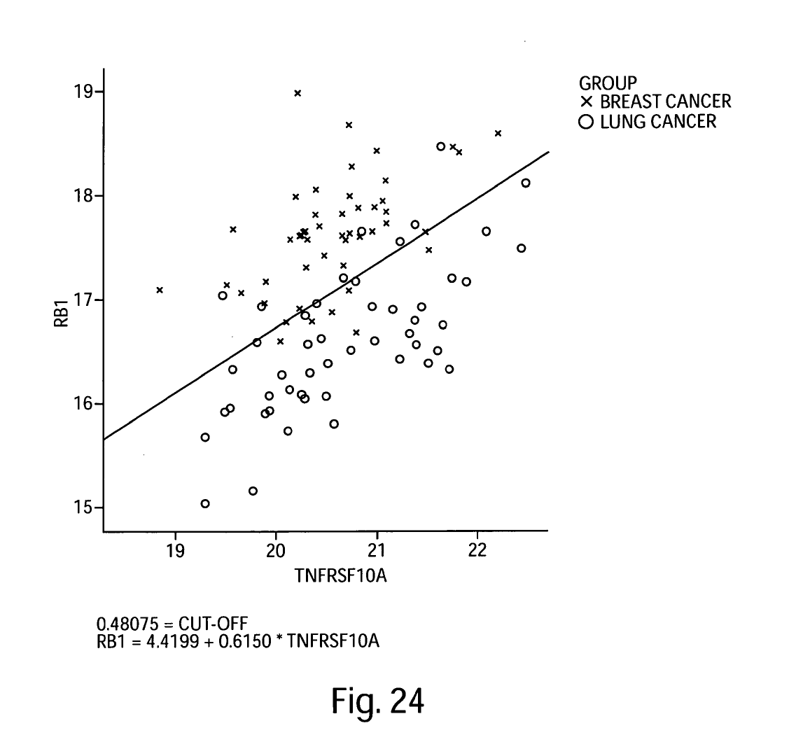

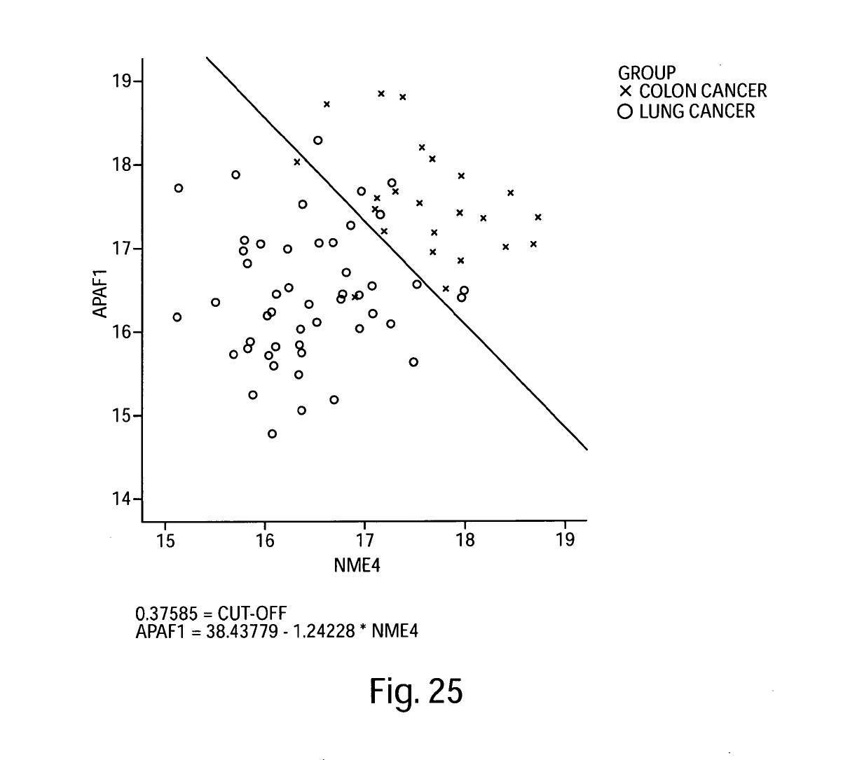

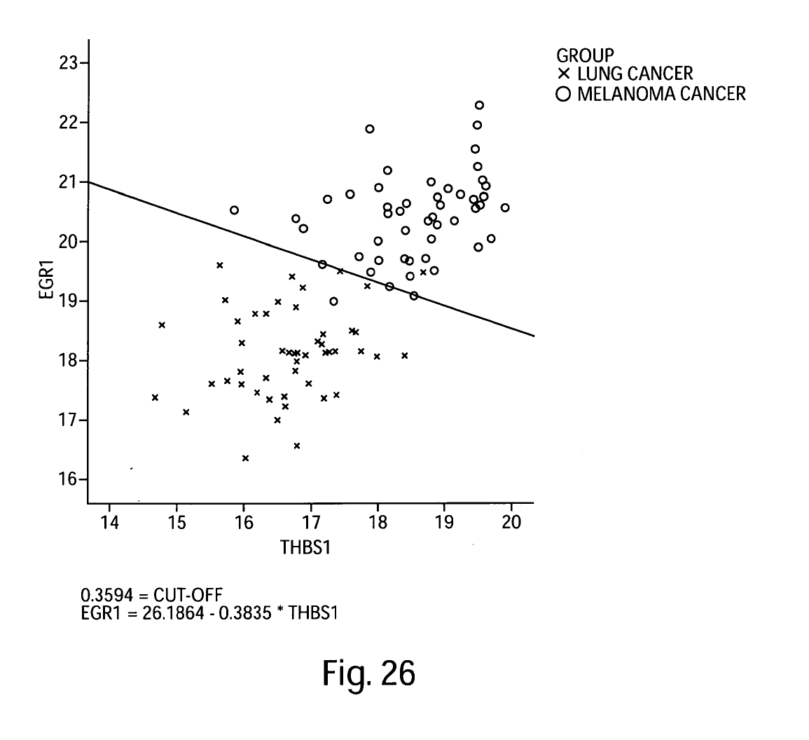

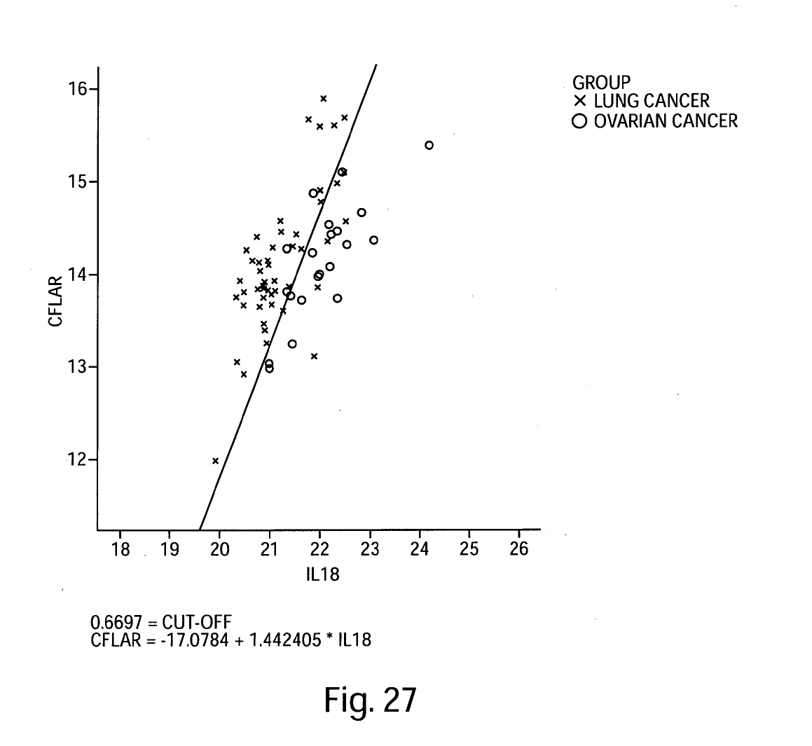

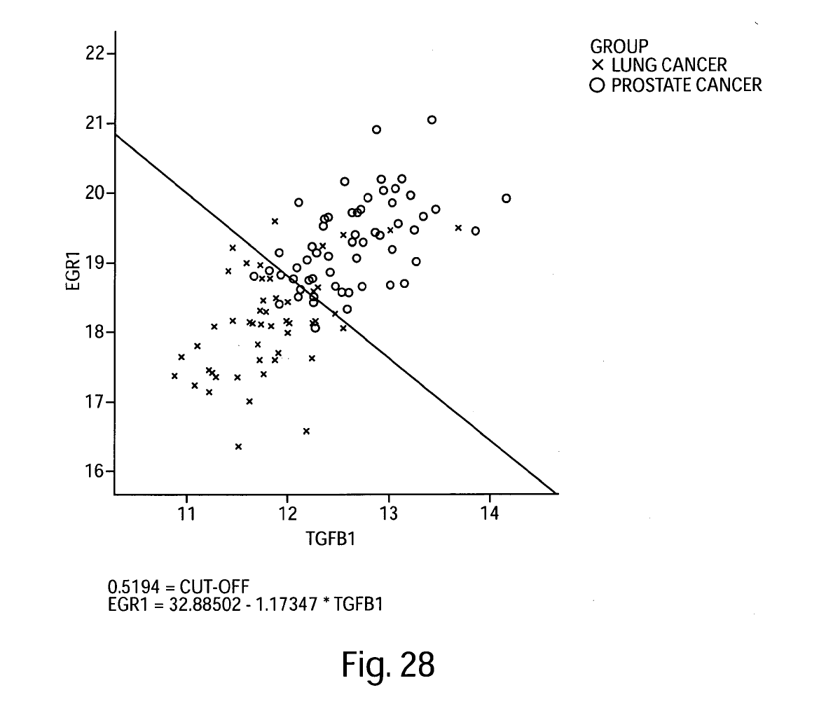

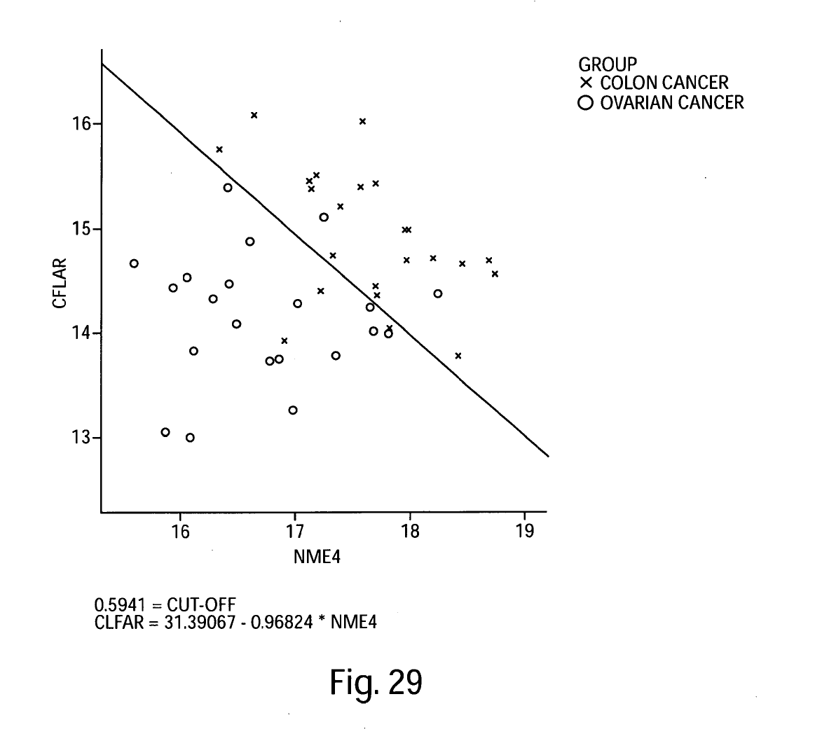

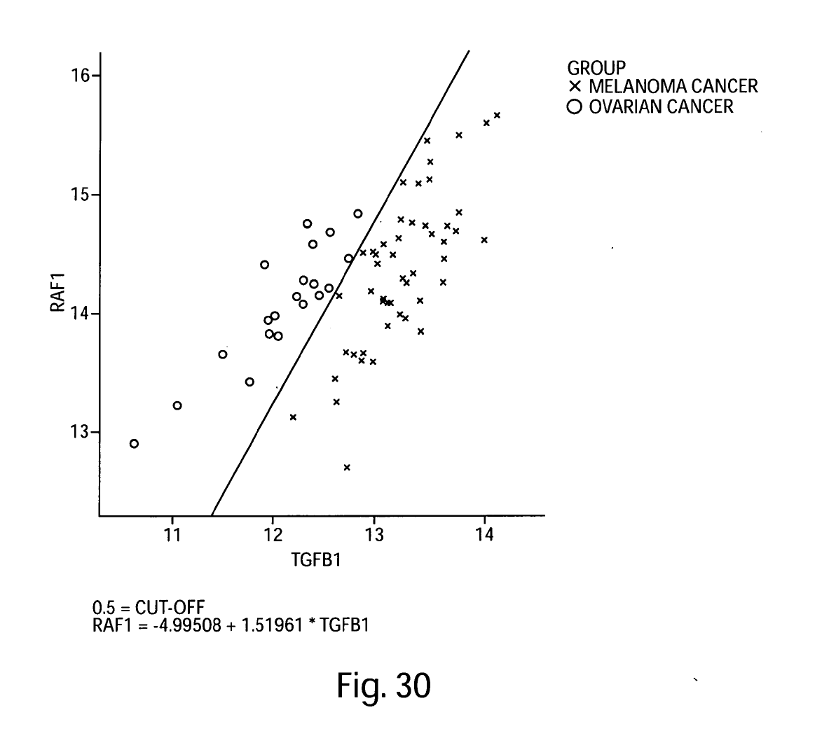

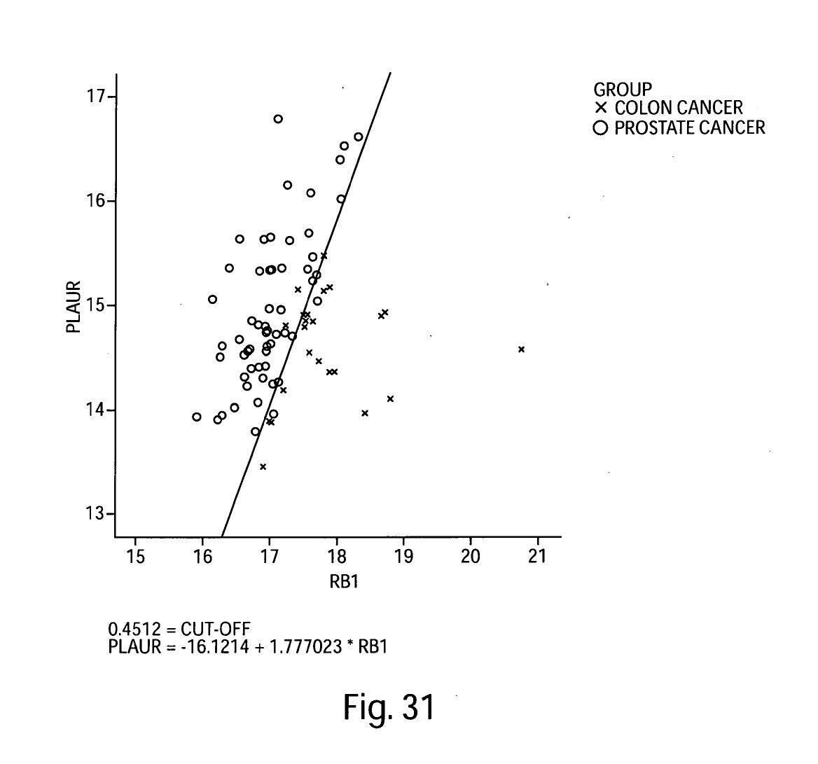

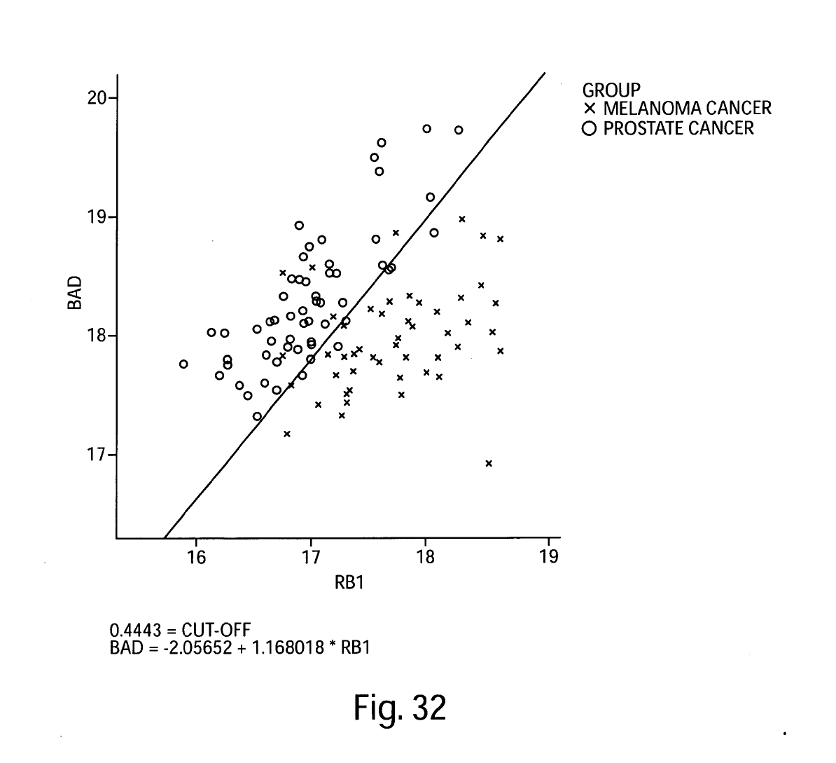

FIGS. 18-32 are discrimination plots based on the Human Cancer General Precision Profile™ capable of distinguishing between Cancer A vs. Cancer B with at least 75% accuracy, for some of the “best” 2-gene models listed in Tables B1a-B18a, as described above in the ‘Brief Description of the Drawings’. For example, FIG. 18 is a graphical representation of the “best” logistic regression model, RAF1 and TGFB1 (identified in Table B1a), based on the Human Cancer General Precision Profile™ (Table B), capable of distinguishing between subjects afflicted with breast cancer and subjects afflicted with melanoma (active disease, stages 2-4). The discrimination line appended to FIG. 18 illustrates how well the 2-gene model discriminates between the 2 groups. Values to the left of the line represent subjects predicted to be in the breast cancer population. Values to the right of the line represent subjects predicted to be in the melanoma population. As shown in FIG. 18, 4 breast cancer subjects (X's) and three melanoma subjects (circles) are classified in the wrong patient population.

The cut-off value used to generate the discrimination line and the line equation are shown below FIGS. 18-32, respectively. The slope and intercept of the discrimination lines were determined as previously described in Example 2. For example, the equation for the discrimination line shown in FIG. 18 is:

RAF1=−13.87+2.19*TGFB1

The intercept (alpha) and slope (beta) of the discrimination line was computed as follows: A cutoff of 0.4871 was used to compute alpha (equals −0.05161 logit units).

The intercept C₀=−13.87 was computed by taking the difference between the intercepts for the 2 groups [32.7734−(−32.7734)=65.5468] and subtracting the log-odds of the cutoff probability (−0.05161). This quantity was then multiplied by −1/X where X is the coefficient for RAF1 (4.7278).

A ranking of the top 79 genes for which gene expression profiles were obtained, from most to least significant, is shown in Tables B1b-B18b. Tables B1b-B18b summarizes the results of significance tests (p-values) for the difference in the mean expression levels for Cancer A subjects and Cancer B subjects, for each of the 18 cancer vs. cancer comparisons, respectively.

In some instances, also provided are the expression values (ΔC_{T}) for each of the Cancer A and Cancer B subjects used to analyze the “best” gene model (after exclusion of missing values) and their predicted probability of having Cancer A vs. Cancer B, as shown in Tables B1c-B8c, and B10c-B17c. For example, as shown in Table B1c, the predicted probability of a subject having breast cancer versus melanoma (active disease, stages 2-4), based on the 2-gene model RAF 1 and TGFB1 (identified in Table B1a) is based on a scale of 0 to 1, “0” indicating the subject has melanoma (active disease, stages 2-4) “1” indicating the subject has breast cancer. This predicted probability can be used to create an index based on the 2-gene model ALOX5 and PLAUR that can be used as a tool by a practitioner (e.g., primary care physician, oncologist, etc.) for diagnosis of breast cancer versus melanoma (active disease, stages 2-4), and to ascertain the necessity of future screening or treatment options.

### Example 5

### EGR1 Precision Profile™

Custom primers and probes were prepared for the targeted 39 genes shown in the Precision Profile™ for EGR1 (shown in Table C), selected to be informative of the biological role early growth response genes play in human cancer (including but not limited to ovarian, breast, cervical, prostate, lung, colon, and skin cancer). Gene expression profiles for these 39 genes were analyzed using the RNA samples obtained from the melanoma (N=49, stages 2-4, active disease), lung cancer (N=49, all stages), colon cancer (N=22), prostate cancer (N=57, all stages), ovarian cancer (N=21, all stages), breast cancer (N=48, all stages), and cervical cancer (N=24, all stages) subjects, described in Example 1, to compare one type of cancer (Cancer A) to another type of cancer (Cancer B). The following 17 combinations of cancer versus cancer comparisons were analyzed to identify logistic regression gene-models based on the EGR1 Precision Profile™ (Table C) capable of distinguishing between subjects having one type of cancer (i.e., Cancer A) versus subjects having another type of cancer (i.e., Cancer B): breast cancer vs. melanoma (active disease, stages 2-4); breast cancer vs. ovarian cancer; cervical cancer vs. breast cancer; cervical cancer vs. colon cancer; cervical cancer vs. melanoma (active disease, stages 2-4); cervical cancer vs. ovarian cancer; colon cancer vs. melanoma (active disease, stages 2-4); lung cancer vs. breast cancer; lung cancer vs. cervical cancer; lung cancer vs. colon cancer; lung cancer vs. melanoma (active disease, stages 2-4); lung cancer vs. ovarian cancer; lung cancer vs. prostate cancer; ovarian cancer vs. colon cancer; ovarian cancer vs. melanoma (active disease, stages 2-4); prostate cancer vs. colon cancer; and prostate cancer vs. melanoma (active disease, stages 2-4).

Logistic regression models yielding the best discrimination between subjects diagnosed with one type of cancer (Cancer A) versus another type of cancer (Cancer B) were generated using the enumeration and classification methodology described in Example 2. A listing of all 1 and 2-gene logistic regression models capable of distinguishing between subjects diagnosed with Cancer A and subjects diagnosed with Cancer B with at least 75% accuracy are shown in Tables C1a-C17a, read from left to right.

Table C1a lists all 1 and 2-gene models capable of distinguishing between subjects with breast cancer and melanoma (active disease, stages 2-4) with at least 75% accuracy. Table C2a lists all 1 and 2-gene models capable of distinguishing between subjects with breast cancer and ovarian cancer with at least 75% accuracy. Table C3a lists all 1 and 2-gene models capable of distinguishing between subjects with cervical cancer and breast cancer with at least 75% accuracy. Table C4a lists all 1 and 2-gene models capable of distinguishing between subjects with cervical cancer and colon cancer with at least 75% accuracy. Table C5a lists all 1 and 2-gene models capable of distinguishing between subjects with cervical cancer and melanoma (active disease, stages 2-4) with at least 75% accuracy. Table C6a lists all 2-gene models capable of distinguishing between subjects with cervical cancer and ovarian cancer with at least 75% accuracy. Table C7a lists all 1 and 2-gene models capable of distinguishing between subjects with colon cancer and melanoma (active disease, stages 2-4) with at least 75% accuracy. Table C8a lists all 1 and 2-gene models capable of distinguishing between subjects with lung cancer and breast cancer with at least 75% accuracy. Table C9a lists all 1 and 2-gene models capable of distinguishing between subjects with lung cancer and cervical cancer with at least 75% accuracy. Table C10a lists all 1 and 2-gene models capable of distinguishing between subjects with lung cancer and colon cancer with at least 75% accuracy. Table C11a lists all 1 and 2-gene models capable of distinguishing between subjects with lung cancer and melanoma (active disease, stages 2-4) with at least 75% accuracy. Table C12a lists all 2-gene models capable of distinguishing between subjects with lung cancer and ovarian cancer with at least 75% accuracy. Table C13a lists all 1 and 2-gene models capable of distinguishing between subjects with lung cancer and prostate cancer with at least 75% accuracy. Table C14a lists all 1 and 2-gene models capable of distinguishing between subjects with ovarian cancer and colon cancer with at least 75% accuracy. Table C15a lists all 1 and 2-gene models capable of distinguishing between subjects with ovarian cancer and melanoma (active disease, stages 2-4) with at least 75% accuracy. Table C16a lists all 1 and 2-gene models capable of distinguishing between subjects with prostate cancer and colon cancer with at least 75% accuracy. Table C17a lists all 1 and 2-gene models capable of distinguishing between subjects with prostate cancer and melanoma (active disease, stages 2-4) with at least 75% accuracy.

As shown in Tables C1a-C17a, the 1 and 2-gene models are identified in the first two columns on the left side of each table, ranked by their entropy R² value (shown in column 3, ranked from high to low). The number of subjects correctly classified or misclassified by each 1 or 2-gene model for each patient group (i.e., Cancer A vs. Cancer B) is shown in columns 4-7. The percent Cancer A subjects and Cancer B subjects correctly classified by the corresponding gene model is shown in columns 8 and 9. The incremental p-value for each first and second gene in the 1 or 2-gene model is shown in columns 10-11 (note p-values smaller than 1×10⁻¹⁷ are reported as ‘0’). The total number of RNA samples analyzed in each patient group (i.e., Cancer A vs. Cancer B) after exclusion of missing values, is shown in columns 12-13. The values missing from the total sample number for Cancer A and/or Cancer B subjects shown in columns 12-13 correspond to instances in which values were excluded from the logistic regression analysis due to reagent limitations and/or instances where replicates did not meet quality metrics.

The “best” logistic regression model (defined as the model with the highest entropy R² value, as described in Example 2) based on the 39 genes included in the Precision Profile™ for EGR1 for each of the 17 combinations of cancer vs. cancer comparisons is shown in the first row of Tables C1a-C17a, respectively. For example, the first row of Table C1a lists a 2-gene model, RAF1 and TGFB1, capable of classifying melanoma subjects (active disease, stages 2-4) with 93.9% accuracy, and breast cancer subjects with 93.8% accuracy. All 49 melanoma and all 48 breast cancer RNA samples were analyzed for this 2-gene model, no values were excluded. As shown in Table C1a, this 2-gene model correctly classifies all 46 of the melanoma subjects as being in the melanoma patient population, and misclassifies 3 of the melanoma subjects as being in the breast cancer patient population. This 2-gene model correctly classifies 45 breast cancer subjects as being in the breast cancer patient population, and misclassifies 3 of the breast cancer subjects as being in the melanoma patient population. The p-value for the 1^{st} gene, RAF1, is 1.6E-09, the incremental p-value for the second gene, TGFB1 is smaller than 1×10⁻¹⁷ (reported as 0).

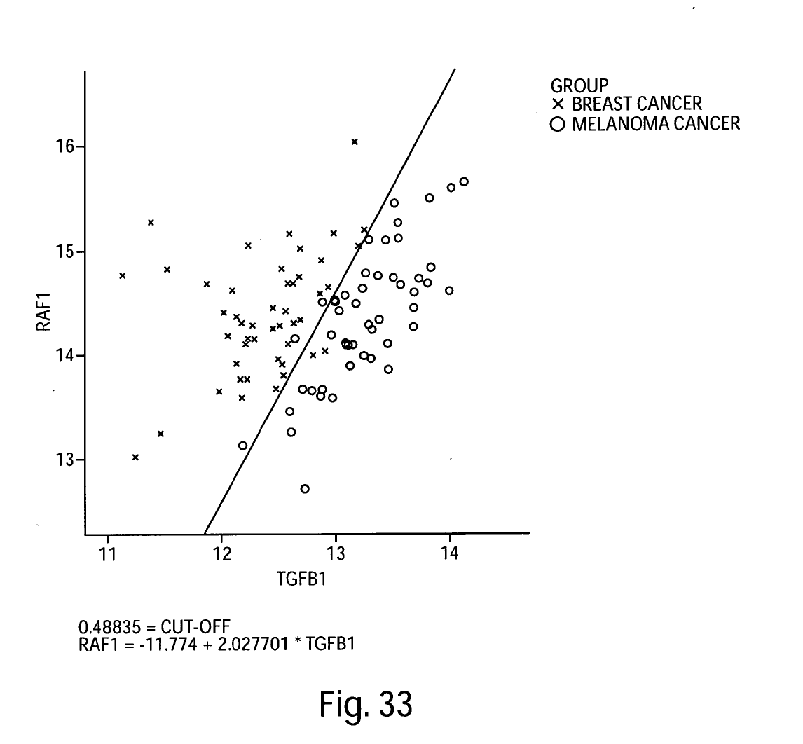

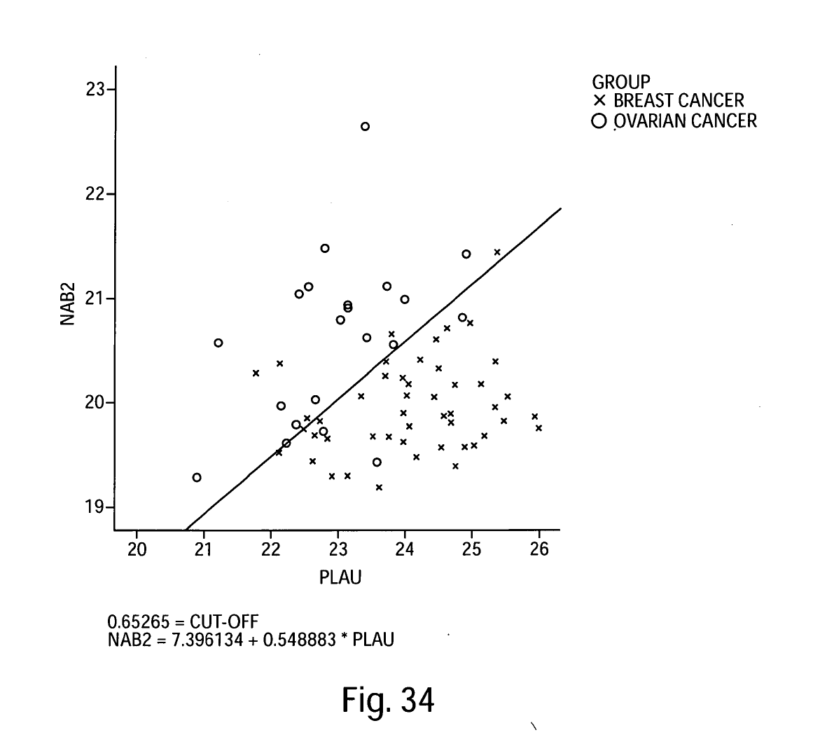

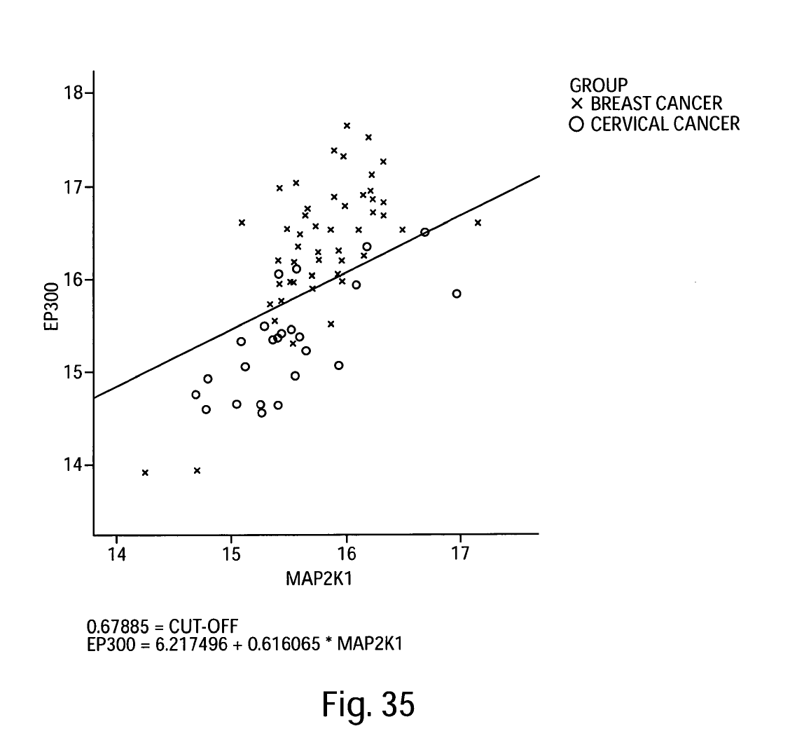

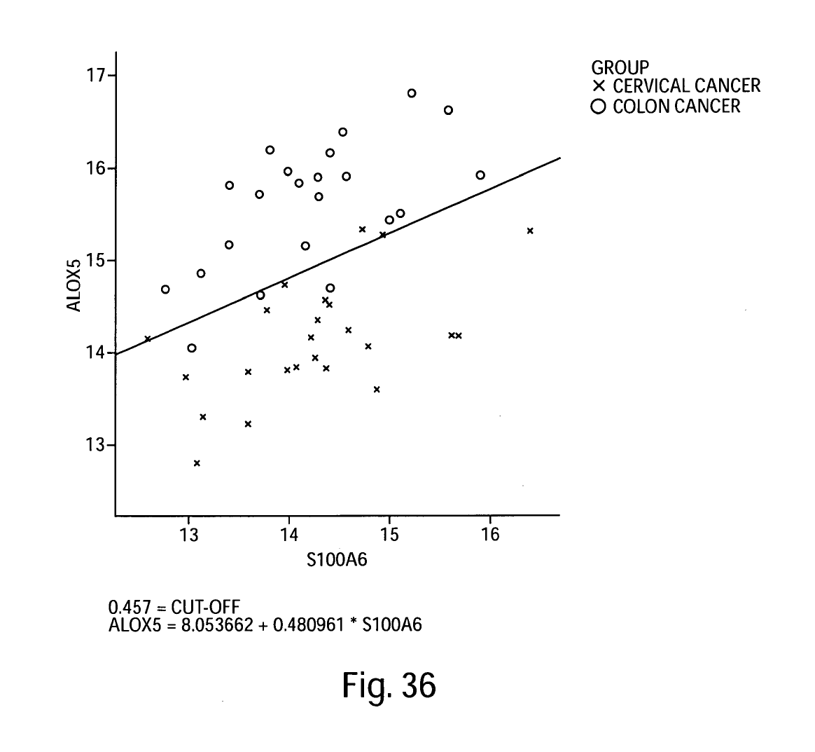

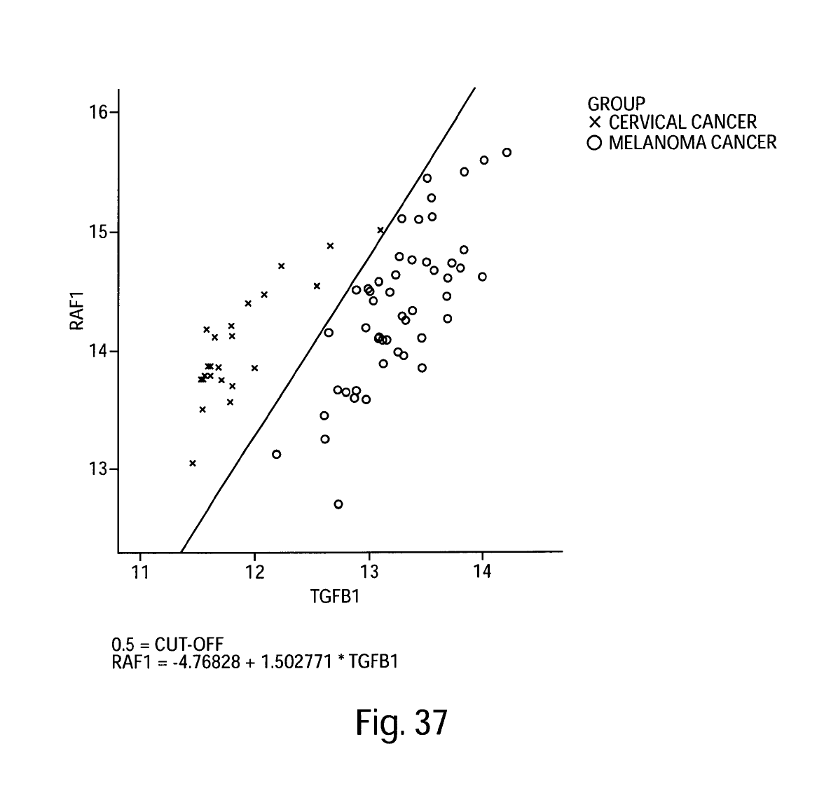

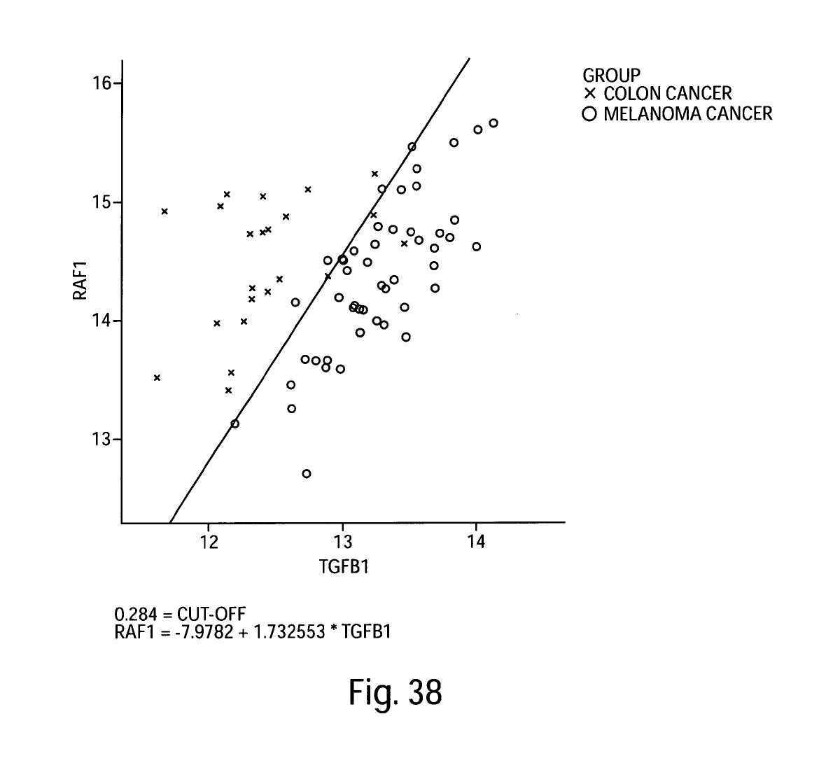

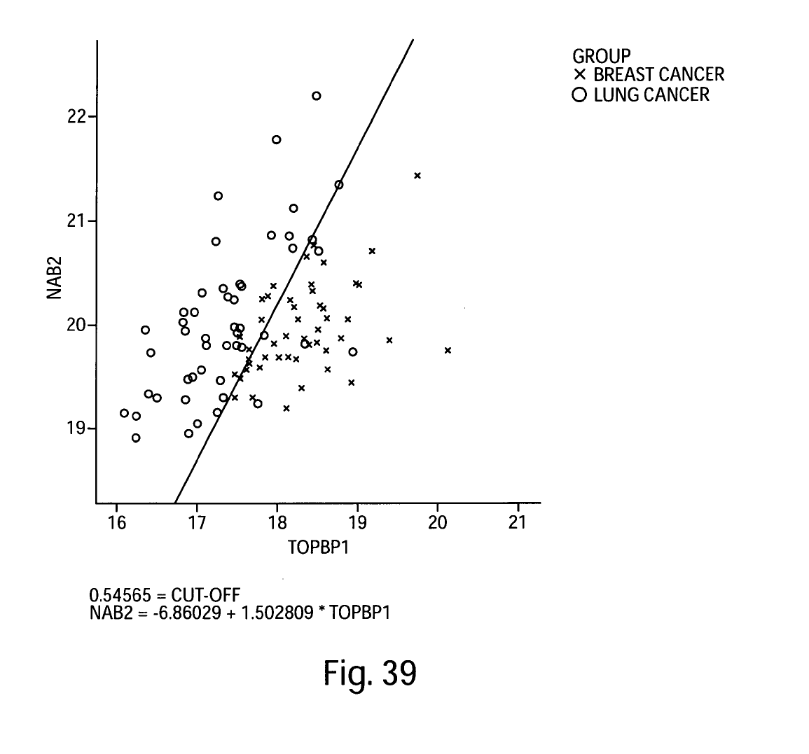

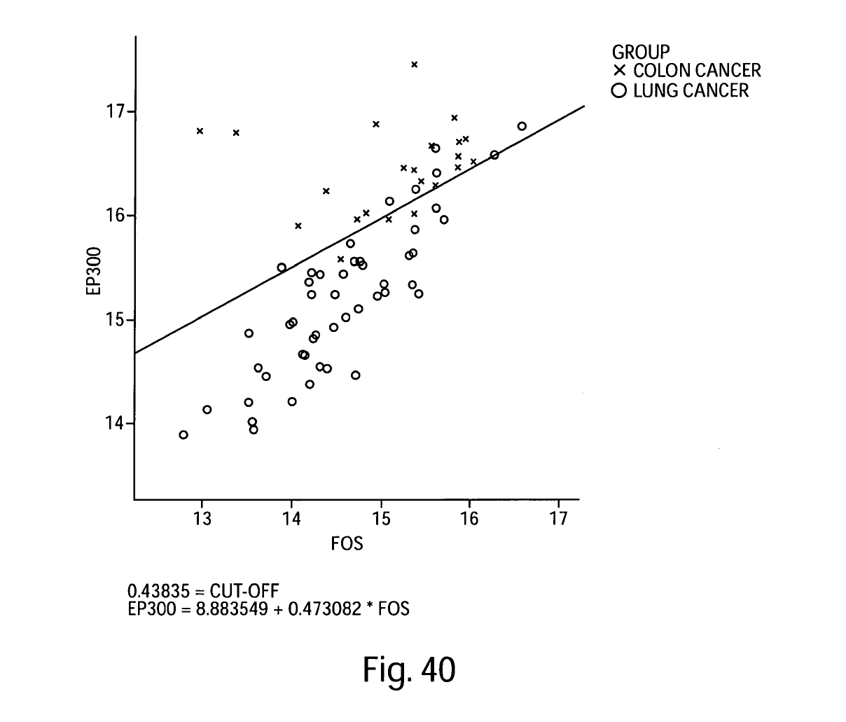

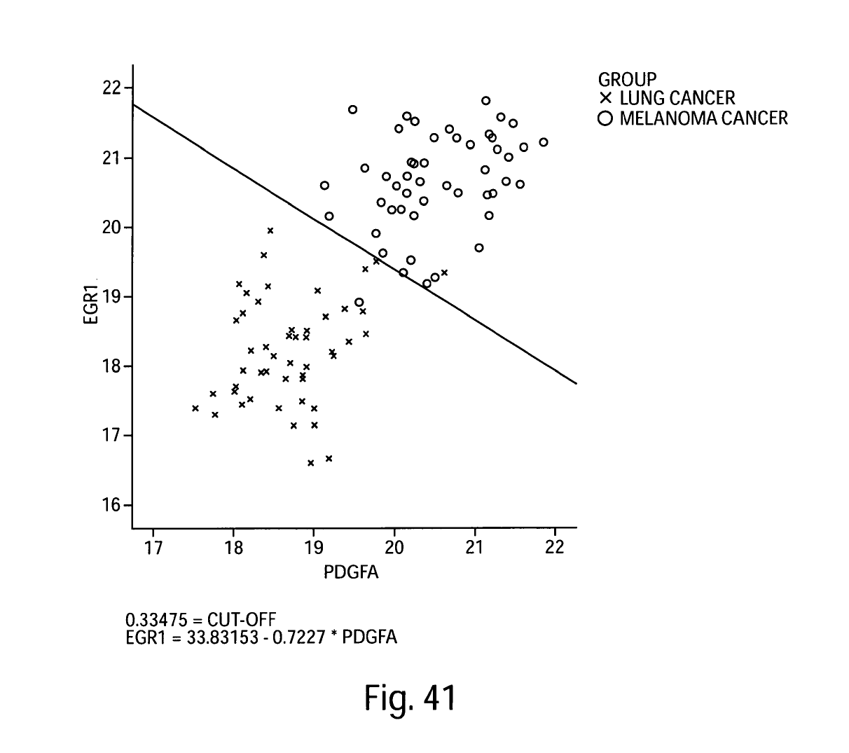

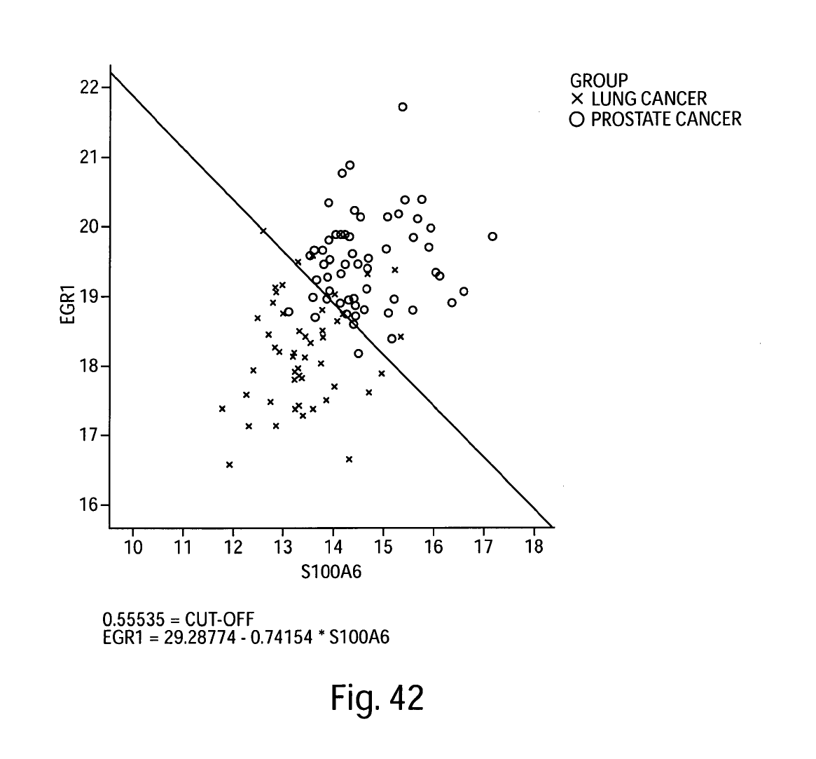

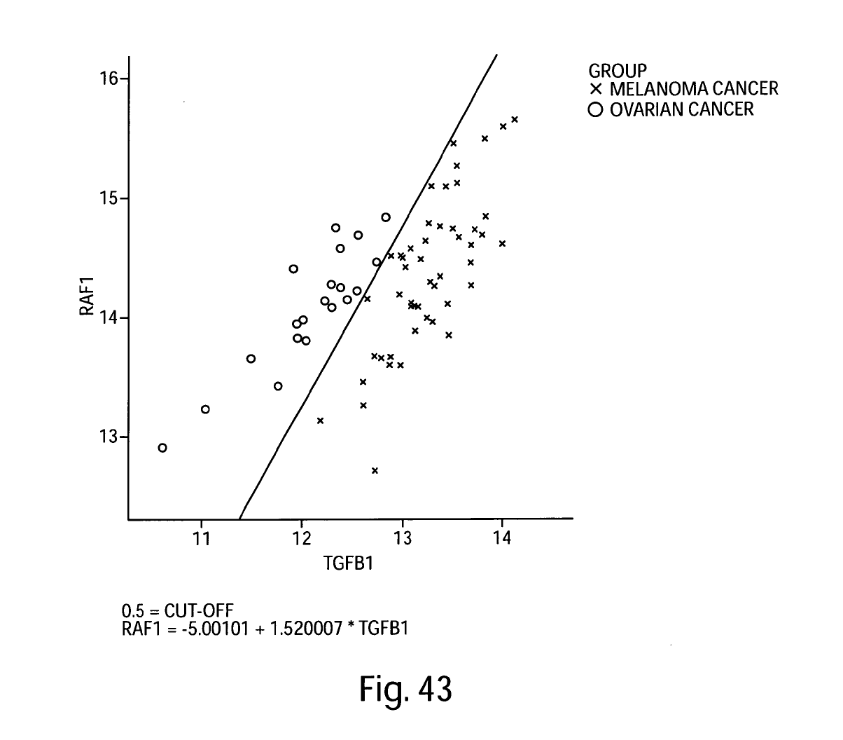

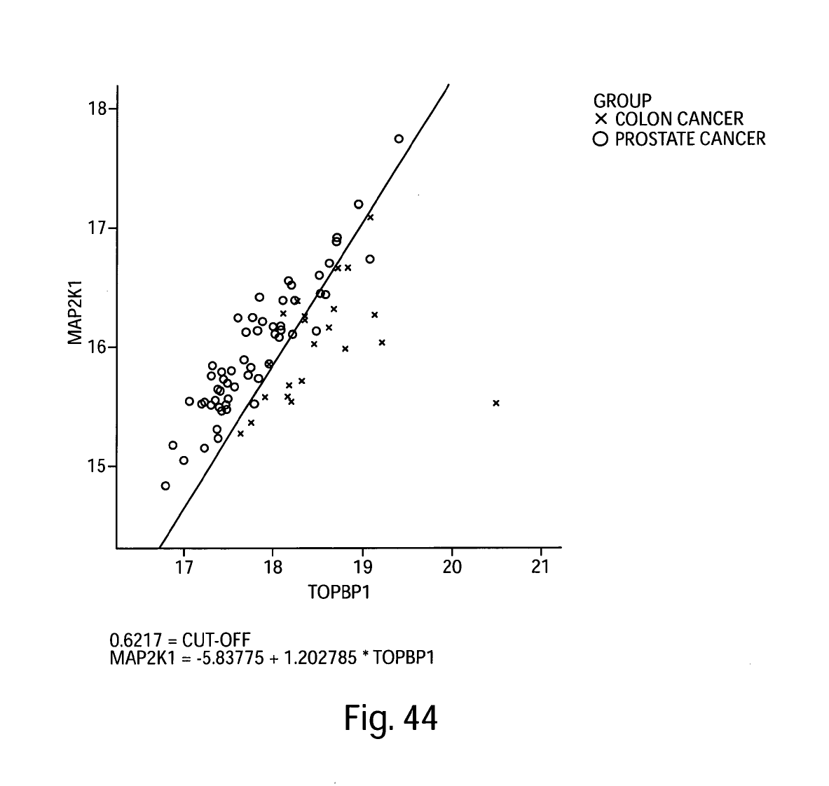

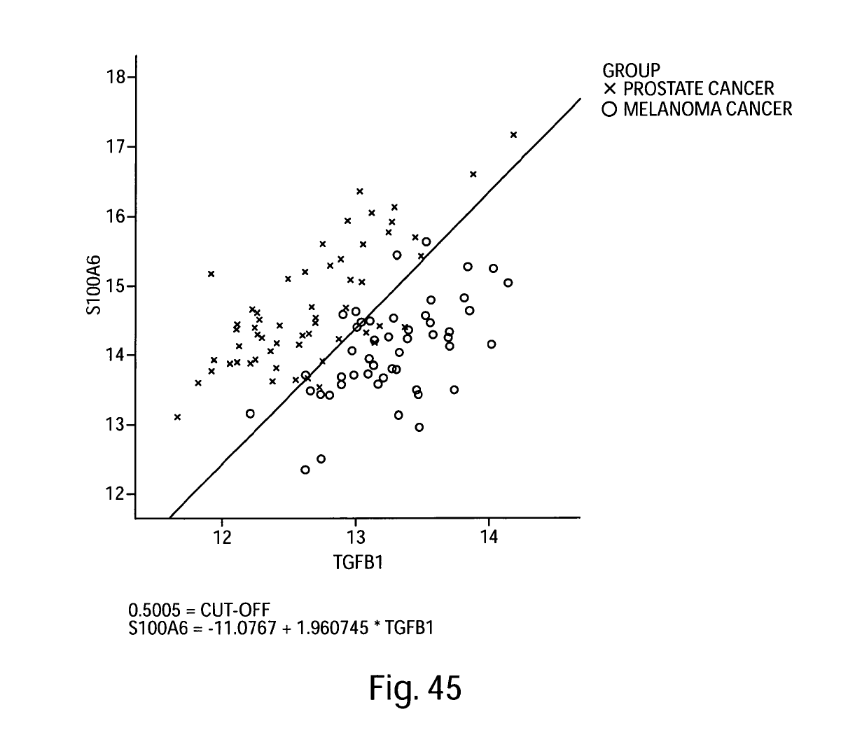

FIGS. 33-45 are discrimination plots based on the Precision Profile™ for EGR1, capable of distinguishing between Cancer A vs. Cancer B with at least 75% accuracy, for some of the “best” 2-gene models listed in Tables C1a-C17a, as described above in the ‘Brief Description of the Drawings’. For example, FIG. 33 is a graphical representation of the “best” logistic regression model, RAF 1 and TGFB1 (identified in Table C1a), based on the Precision Profile™ for EGR1 (Table C), capable of distinguishing between subjects afflicted with breast cancer and subjects afflicted with melanoma (active disease, stages 2-4). The discrimination line appended to FIG. 33 illustrates how well the 2-gene model discriminates between the 2 groups. Values to the left of the line represent subjects predicted to be in the breast cancer population. Values to the right of the line represent subjects predicted to be in the melanoma population. As shown in FIG. 2, 3 breast cancer subjects (X's) and 3 melanoma subjects (all stages) (circles) are classified in the wrong patient population.

The cut-off value used to generate the discrimination line and the line equation are shown below FIGS. 33-45, respectively. The slope and intercept of the discrimination lines were determined as previously described in Example 2. For example, the equation for the discrimination line shown in FIG. 33 is:

RAF1=−11.774+2.027701*TGFB1

The intercept (alpha) and slope (beta) of the discrimination line was computed as follows: A cutoff of 0.48835 was used to compute alpha (equals −0.04661 logit units).

The intercept C₀=−11.774 was computed by taking the difference between the intercepts for the 2 groups [38.1234−(−38.1234)=76.2468] and subtracting the log-odds of the cutoff probability (−0.04661). This quantity was then multiplied by −1/X where X is the coefficient for RAF1 (6.4798).

A ranking of the top 32 genes for which gene expression profiles were obtained, from most to least significant, is shown in Tables C1b-C17b. Tables C1b-C17b summarizes the results of significance tests (p-values) for the difference in the mean expression levels for Cancer A subjects and Cancer B subjects, for each of the 17 cancer vs. cancer comparisons, respectively.

In some instances, also provided are the expression values (ΔC_{T}) for each of the Cancer A and Cancer B subjects used to analyze the “best” gene model (after exclusion of missing values) and their predicted probability of having Cancer A vs. Cancer B, as shown in Tables C1c-C5c, C7c-C8c, C10c-C13c, and C15c-C17c. For example, as shown in Table C1c, the predicted probability of a subject having breast cancer versus melanoma (active disease, stages 2-4), based on the 2-gene model RAF1 and TGFB1 (identified in Table C1a) is based on a scale of 0 to 1, “0” indicating the subject has melanoma (active disease, stages 2-4)) “1” indicating the subject has breast cancer. This predicted probability can be used to create an index based on the 2-gene model ALOX5 and PLAUR that can be used as a tool by a practitioner (e.g., primary care physician, oncologist, etc.) for diagnosis of breast cancer versus melanoma (active disease, stages 2-4), and to ascertain the necessity of future screening or treatment options.

These data support that Gene Expression Profiles with sufficient precision and calibration as described herein (1) can distinguish between subsets of individuals with a known biological condition, particularly between individuals with one type of cancer versus individuals with another type of cancer; (2) may be used to monitor the response of patients to therapy; (3) may be used to assess the efficacy and safety of therapy; and (4) may be used to guide the medical management of a patient by adjusting therapy to bring one or more relevant Gene Expression Profiles closer to a target set of values, which may be normative values or other desired or achievable values.

Gene Expression Profiles are useful for characterization and monitoring of treatment efficacy of individuals with skin, lung, colon, prostate, ovarian, breast, or cervical cancer, or individuals with conditions related to skin, lung, colon, prostate, ovarian, breast, or cervical cancer. Use of the algorithmic and statistical approaches discussed above to achieve such identification and to discriminate in such fashion is within the scope of various embodiments herein.

The references listed below are hereby incorporated herein by reference.

### REFERENCES

Magidson, J. GOLDMineR User's Guide (1998). Belmont, Mass.: Statistical Innovations Inc.
Vermunt and Magidson (2005). Latent GOLD 4.0 Technical Guide, Belmont Mass.: Statistical Innovations.
Vermunt and Magidson (2007). LG-Syntax™ User's Guide: Manual for Latent GOLD® 4.5 Syntax Module, Belmont Mass.: Statistical Innovations.
Vermunt J. K. and J. Magidson. Latent Class Cluster Analysis in (2002) J. A. Hagenaars and A. L. McCutcheon (eds.), Applied Latent Class Analysis, 89-106. Cambridge: Cambridge University Press.
Magidson, J. “Maximum Likelihood Assessment of Clinical Trials Based on an Ordered Categorical Response.” (1996) Drug Information Journal, Maple Glen, Pa.: Drug Information Association, Vol. 30, No. 1, pp 143-170.

Lengthy table referenced here
US20110097717A1-20110428-T00001
Please refer to the end of the specification for access instructions.

Lengthy table referenced here
US20110097717A1-20110428-T00002
Please refer to the end of the specification for access instructions.

Lengthy table referenced here
US20110097717A1-20110428-T00003
Please refer to the end of the specification for access instructions.

Lengthy table referenced here
US20110097717A1-20110428-T00004
Please refer to the end of the specification for access instructions.

Lengthy table referenced here
US20110097717A1-20110428-T00005
Please refer to the end of the specification for access instructions.

Lengthy table referenced here
US20110097717A1-20110428-T00006
Please refer to the end of the specification for access instructions.

Lengthy table referenced here
US20110097717A1-20110428-T00007
Please refer to the end of the specification for access instructions.

Lengthy table referenced here
US20110097717A1-20110428-T00008
Please refer to the end of the specification for access instructions.

Lengthy table referenced here
US20110097717A1-20110428-T00009
Please refer to the end of the specification for access instructions.

Lengthy table referenced here
US20110097717A1-20110428-T00010
Please refer to the end of the specification for access instructions.

Lengthy table referenced here
US20110097717A1-20110428-T00011
Please refer to the end of the specification for access instructions.

Lengthy table referenced here
US20110097717A1-20110428-T00012
Please refer to the end of the specification for access instructions.

Lengthy table referenced here
US20110097717A1-20110428-T00013
Please refer to the end of the specification for access instructions.

Lengthy table referenced here
US20110097717A1-20110428-T00014
Please refer to the end of the specification for access instructions.

Lengthy table referenced here
US20110097717A1-20110428-T00015
Please refer to the end of the specification for access instructions.

Lengthy table referenced here
US20110097717A1-20110428-T00016
Please refer to the end of the specification for access instructions.

Lengthy table referenced here
US20110097717A1-20110428-T00017
Please refer to the end of the specification for access instructions.

Lengthy table referenced here
US20110097717A1-20110428-T00018
Please refer to the end of the specification for access instructions.

Lengthy table referenced here
US20110097717A1-20110428-T00019
Please refer to the end of the specification for access instructions.

Lengthy table referenced here
US20110097717A1-20110428-T00020
Please refer to the end of the specification for access instructions.

Lengthy table referenced here
US20110097717A1-20110428-T00021
Please refer to the end of the specification for access instructions.

Lengthy table referenced here
US20110097717A1-20110428-T00022
Please refer to the end of the specification for access instructions.

Lengthy table referenced here
US20110097717A1-20110428-T00023
Please refer to the end of the specification for access instructions.

Lengthy table referenced here
US20110097717A1-20110428-T00024
Please refer to the end of the specification for access instructions.

Lengthy table referenced here
US20110097717A1-20110428-T00025
Please refer to the end of the specification for access instructions.

Lengthy table referenced here
US20110097717A1-20110428-T00026
Please refer to the end of the specification for access instructions.

Lengthy table referenced here
US20110097717A1-20110428-T00027
Please refer to the end of the specification for access instructions.

Lengthy table referenced here
US20110097717A1-20110428-T00028
Please refer to the end of the specification for access instructions.

Lengthy table referenced here
US20110097717A1-20110428-T00029
Please refer to the end of the specification for access instructions.

Lengthy table referenced here
US20110097717A1-20110428-T00030
Please refer to the end of the specification for access instructions.

Lengthy table referenced here
US20110097717A1-20110428-T00031
Please refer to the end of the specification for access instructions.

Lengthy table referenced here
US20110097717A1-20110428-T00032
Please refer to the end of the specification for access instructions.

Lengthy table referenced here
US20110097717A1-20110428-T00033
Please refer to the end of the specification for access instructions.

Lengthy table referenced here
US20110097717A1-20110428-T00034
Please refer to the end of the specification for access instructions.

Lengthy table referenced here
US20110097717A1-20110428-T00035
Please refer to the end of the specification for access instructions.

Lengthy table referenced here
US20110097717A1-20110428-T00036
Please refer to the end of the specification for access instructions.

Lengthy table referenced here
US20110097717A1-20110428-T00037
Please refer to the end of the specification for access instructions.

Lengthy table referenced here
US20110097717A1-20110428-T00038
Please refer to the end of the specification for access instructions.

Lengthy table referenced here
US20110097717A1-20110428-T00039
Please refer to the end of the specification for access instructions.

Lengthy table referenced here
US20110097717A1-20110428-T00040
Please refer to the end of the specification for access instructions.

Lengthy table referenced here
US20110097717A1-20110428-T00041
Please refer to the end of the specification for access instructions.

Lengthy table referenced here
US20110097717A1-20110428-T00042
Please refer to the end of the specification for access instructions.

Lengthy table referenced here
US20110097717A1-20110428-T00043
Please refer to the end of the specification for access instructions.

Lengthy table referenced here
US20110097717A1-20110428-T00044
Please refer to the end of the specification for access instructions.

Lengthy table referenced here
US20110097717A1-20110428-T00045
Please refer to the end of the specification for access instructions.

Lengthy table referenced here
US20110097717A1-20110428-T00046
Please refer to the end of the specification for access instructions.

Lengthy table referenced here
US20110097717A1-20110428-T00047
Please refer to the end of the specification for access instructions.

Lengthy table referenced here
US20110097717A1-20110428-T00048
Please refer to the end of the specification for access instructions.

Lengthy table referenced here
US20110097717A1-20110428-T00049
Please refer to the end of the specification for access instructions.

Lengthy table referenced here
US20110097717A1-20110428-T00050
Please refer to the end of the specification for access instructions.

Lengthy table referenced here
US20110097717A1-20110428-T00051
Please refer to the end of the specification for access instructions.

Lengthy table referenced here
US20110097717A1-20110428-T00052
Please refer to the end of the specification for access instructions.

Lengthy table referenced here
US20110097717A1-20110428-T00053
Please refer to the end of the specification for access instructions.

Lengthy table referenced here
US20110097717A1-20110428-T00054
Please refer to the end of the specification for access instructions.

Lengthy table referenced here
US20110097717A1-20110428-T00055
Please refer to the end of the specification for access instructions.

Lengthy table referenced here
US20110097717A1-20110428-T00056
Please refer to the end of the specification for access instructions.

Lengthy table referenced here
US20110097717A1-20110428-T00057
Please refer to the end of the specification for access instructions.

Lengthy table referenced here
US20110097717A1-20110428-T00058
Please refer to the end of the specification for access instructions.

Lengthy table referenced here
US20110097717A1-20110428-T00059
Please refer to the end of the specification for access instructions.

Lengthy table referenced here
US20110097717A1-20110428-T00060
Please refer to the end of the specification for access instructions.

Lengthy table referenced here
US20110097717A1-20110428-T00061
Please refer to the end of the specification for access instructions.

Lengthy table referenced here
US20110097717A1-20110428-T00062
Please refer to the end of the specification for access instructions.

Lengthy table referenced here
US20110097717A1-20110428-T00063
Please refer to the end of the specification for access instructions.

Lengthy table referenced here
US20110097717A1-20110428-T00064
Please refer to the end of the specification for access instructions.

Lengthy table referenced here
US20110097717A1-20110428-T00065
Please refer to the end of the specification for access instructions.

Lengthy table referenced here
US20110097717A1-20110428-T00066
Please refer to the end of the specification for access instructions.

Lengthy table referenced here
US20110097717A1-20110428-T00067
Please refer to the end of the specification for access instructions.

Lengthy table referenced here
US20110097717A1-20110428-T00068
Please refer to the end of the specification for access instructions.

Lengthy table referenced here
US20110097717A1-20110428-T00069
Please refer to the end of the specification for access instructions.

Lengthy table referenced here
US20110097717A1-20110428-T00070
Please refer to the end of the specification for access instructions.

Lengthy table referenced here
US20110097717A1-20110428-T00071
Please refer to the end of the specification for access instructions.

Lengthy table referenced here
US20110097717A1-20110428-T00072
Please refer to the end of the specification for access instructions.

Lengthy table referenced here
US20110097717A1-20110428-T00073
Please refer to the end of the specification for access instructions.

Lengthy table referenced here
US20110097717A1-20110428-T00074
Please refer to the end of the specification for access instructions.

Lengthy table referenced here
US20110097717A1-20110428-T00075
Please refer to the end of the specification for access instructions.

Lengthy table referenced here
US20110097717A1-20110428-T00076
Please refer to the end of the specification for access instructions.

Lengthy table referenced here
US20110097717A1-20110428-T00077
Please refer to the end of the specification for access instructions.

Lengthy table referenced here
US20110097717A1-20110428-T00078
Please refer to the end of the specification for access instructions.

Lengthy table referenced here
US20110097717A1-20110428-T00079
Please refer to the end of the specification for access instructions.

Lengthy table referenced here
US20110097717A1-20110428-T00080
Please refer to the end of the specification for access instructions.

Lengthy table referenced here
US20110097717A1-20110428-T00081
Please refer to the end of the specification for access instructions.

Lengthy table referenced here
US20110097717A1-20110428-T00082
Please refer to the end of the specification for access instructions.

Lengthy table referenced here
US20110097717A1-20110428-T00083
Please refer to the end of the specification for access instructions.

Lengthy table referenced here
US20110097717A1-20110428-T00084
Please refer to the end of the specification for access instructions.

Lengthy table referenced here
US20110097717A1-20110428-T00085
Please refer to the end of the specification for access instructions.

Lengthy table referenced here
US20110097717A1-20110428-T00086
Please refer to the end of the specification for access instructions.

Lengthy table referenced here
US20110097717A1-20110428-T00087
Please refer to the end of the specification for access instructions.

Lengthy table referenced here
US20110097717A1-20110428-T00088
Please refer to the end of the specification for access instructions.

Lengthy table referenced here
US20110097717A1-20110428-T00089
Please refer to the end of the specification for access instructions.

Lengthy table referenced here
US20110097717A1-20110428-T00090
Please refer to the end of the specification for access instructions.

Lengthy table referenced here
US20110097717A1-20110428-T00091
Please refer to the end of the specification for access instructions.

Lengthy table referenced here
US20110097717A1-20110428-T00092
Please refer to the end of the specification for access instructions.

Lengthy table referenced here
US20110097717A1-20110428-T00093
Please refer to the end of the specification for access instructions.

Lengthy table referenced here
US20110097717A1-20110428-T00094
Please refer to the end of the specification for access instructions.

Lengthy table referenced here
US20110097717A1-20110428-T00095
Please refer to the end of the specification for access instructions.

Lengthy table referenced here
US20110097717A1-20110428-T00096
Please refer to the end of the specification for access instructions.

Lengthy table referenced here
US20110097717A1-20110428-T00097
Please refer to the end of the specification for access instructions.

Lengthy table referenced here
US20110097717A1-20110428-T00098
Please refer to the end of the specification for access instructions.

Lengthy table referenced here
US20110097717A1-20110428-T00099
Please refer to the end of the specification for access instructions.

Lengthy table referenced here
US20110097717A1-20110428-T00100
Please refer to the end of the specification for access instructions.

Lengthy table referenced here
US20110097717A1-20110428-T00101
Please refer to the end of the specification for access instructions.

Lengthy table referenced here
US20110097717A1-20110428-T00102
Please refer to the end of the specification for access instructions.

Lengthy table referenced here
US20110097717A1-20110428-T00103
Please refer to the end of the specification for access instructions.

Lengthy table referenced here
US20110097717A1-20110428-T00104
Please refer to the end of the specification for access instructions.

Lengthy table referenced here
US20110097717A1-20110428-T00105
Please refer to the end of the specification for access instructions.

Lengthy table referenced here
US20110097717A1-20110428-T00106
Please refer to the end of the specification for access instructions.

Lengthy table referenced here
US20110097717A1-20110428-T00107
Please refer to the end of the specification for access instructions.

Lengthy table referenced here
US20110097717A1-20110428-T00108
Please refer to the end of the specification for access instructions.

Lengthy table referenced here
US20110097717A1-20110428-T00109
Please refer to the end of the specification for access instructions.

Lengthy table referenced here
US20110097717A1-20110428-T00110
Please refer to the end of the specification for access instructions.

Lengthy table referenced here
US20110097717A1-20110428-T00111
Please refer to the end of the specification for access instructions.

Lengthy table referenced here
US20110097717A1-20110428-T00112
Please refer to the end of the specification for access instructions.

Lengthy table referenced here
US20110097717A1-20110428-T00113
Please refer to the end of the specification for access instructions.

Lengthy table referenced here
US20110097717A1-20110428-T00114
Please refer to the end of the specification for access instructions.

Lengthy table referenced here
US20110097717A1-20110428-T00115
Please refer to the end of the specification for access instructions.

Lengthy table referenced here
US20110097717A1-20110428-T00116
Please refer to the end of the specification for access instructions.

Lengthy table referenced here
US20110097717A1-20110428-T00117
Please refer to the end of the specification for access instructions.

Lengthy table referenced here
US20110097717A1-20110428-T00118
Please refer to the end of the specification for access instructions.

Lengthy table referenced here
US20110097717A1-20110428-T00119
Please refer to the end of the specification for access instructions.

Lengthy table referenced here
US20110097717A1-20110428-T00120
Please refer to the end of the specification for access instructions.

Lengthy table referenced here
US20110097717A1-20110428-T00121
Please refer to the end of the specification for access instructions.

Lengthy table referenced here
US20110097717A1-20110428-T00122
Please refer to the end of the specification for access instructions.

Lengthy table referenced here
US20110097717A1-20110428-T00123
Please refer to the end of the specification for access instructions.

Lengthy table referenced here
US20110097717A1-20110428-T00124
Please refer to the end of the specification for access instructions.

Lengthy table referenced here
US20110097717A1-20110428-T00125
Please refer to the end of the specification for access instructions.

Lengthy table referenced here
US20110097717A1-20110428-T00126
Please refer to the end of the specification for access instructions.

Lengthy table referenced here
US20110097717A1-20110428-T00127
Please refer to the end of the specification for access instructions.

Lengthy table referenced here
US20110097717A1-20110428-T00128
Please refer to the end of the specification for access instructions.

Lengthy table referenced here
US20110097717A1-20110428-T00129
Please refer to the end of the specification for access instructions.

Lengthy table referenced here
US20110097717A1-20110428-T00130
Please refer to the end of the specification for access instructions.

Lengthy table referenced here
US20110097717A1-20110428-T00131
Please refer to the end of the specification for access instructions.

Lengthy table referenced here
US20110097717A1-20110428-T00132
Please refer to the end of the specification for access instructions.

Lengthy table referenced here
US20110097717A1-20110428-T00133
Please refer to the end of the specification for access instructions.

Lengthy table referenced here
US20110097717A1-20110428-T00134
Please refer to the end of the specification for access instructions.

Lengthy table referenced here
US20110097717A1-20110428-T00135
Please refer to the end of the specification for access instructions.

Lengthy table referenced here
US20110097717A1-20110428-T00136
Please refer to the end of the specification for access instructions.

Lengthy table referenced here
US20110097717A1-20110428-T00137
Please refer to the end of the specification for access instructions.

Lengthy table referenced here
US20110097717A1-20110428-T00138
Please refer to the end of the specification for access instructions.

Lengthy table referenced here
US20110097717A1-20110428-T00139
Please refer to the end of the specification for access instructions.

Lengthy table referenced here
US20110097717A1-20110428-T00140
Please refer to the end of the specification for access instructions.

Lengthy table referenced here
US20110097717A1-20110428-T00141
Please refer to the end of the specification for access instructions.

Lengthy table referenced here
US20110097717A1-20110428-T00142
Please refer to the end of the specification for access instructions.

Lengthy table referenced here
US20110097717A1-20110428-T00143
Please refer to the end of the specification for access instructions.

Lengthy table referenced here
US20110097717A1-20110428-T00144
Please refer to the end of the specification for access instructions.

Lengthy table referenced here
US20110097717A1-20110428-T00145
Please refer to the end of the specification for access instructions.

Lengthy table referenced here
US20110097717A1-20110428-T00146
Please refer to the end of the specification for access instructions.

Lengthy table referenced here
US20110097717A1-20110428-T00147
Please refer to the end of the specification for access instructions.

Lengthy table referenced here
US20110097717A1-20110428-T00148
Please refer to the end of the specification for access instructions.

Lengthy table referenced here
US20110097717A1-20110428-T00149
Please refer to the end of the specification for access instructions.

Lengthy table referenced here
US20110097717A1-20110428-T00150
Please refer to the end of the specification for access instructions.

Lengthy table referenced here
US20110097717A1-20110428-T00151
Please refer to the end of the specification for access instructions.

Lengthy table referenced here
US20110097717A1-20110428-T00152
Please refer to the end of the specification for access instructions.

Lengthy table referenced here
US20110097717A1-20110428-T00153
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110097717A1). An electronic copy of the table will also be available from the USPTO upon request and payment of the fee set forth in 37 CFR 1.19(b)(3).

1. A method for evaluating the presence of breast cancer in a subject based on a sample from the subject, the sample providing a source of RNAs, comprising:
a) determining a quantitative measure of the amount of at least one constituent of any constituent of any one table selected from the group consisting of Tables A, B and C, as a distinct RNA constituent in the subject sample, wherein such measure is obtained under measurement conditions that are substantially repeatable and the constituent is selected so that measurement of the constituent distinguishes between a breast cancer diagnosed subject and a subject having a cancer selected from the group consisting of melanoma, lung, colon, ovarian and cervical in a reference population with at least 75% accuracy.
b) comparing the quantitative measure of the constituent in the subject sample to a reference value.

2. The method of claim 1, wherein said constituent is selected from Table A and is
a) LTA, IFI16, PTPRC, CD86, ADAM17, HMOX1, TXNRD1, MYC, MHC2TA, MAPK14, TLR2, CD19, TNFRSF1A, TIMP1, TNF, IL23A, HLADRA, TLR4, PLAUR, PTGS2, PLA2G7, CCR5, or TOSO wherein the constituent distinguishes between a breast cancer diagnosed subject and a colon cancer diagnosed subject in a reference population with at least 75% accuracy;
b) IFI16, TIMP1, MAPK14, LTA, TGFB1, HMOX1, TNFRSF1A, PTPRC, PLAUR, EGR1, ADAM17, TLR2, MYC, SSI3, TNF, CD86, IL1B, CCL5, MHC2TA, CXCR3, TXNRD1, PTGS2, ICAM1, IL1RN, SERPINE1, CD4, NFKB1, CCR5, TLR4, IL18BP, CCL3, HLADRA, MMP9, or IL32 wherein the constituent distinguishes between a breast cancer diagnosed subject and a melanoma cancer diagnosed subject in a reference population with at least 75% accuracy;
c) TIMP1, MAPK14, SSI3, PTPRC, or IL1RN wherein the constituent distinguishes between a breast cancer diagnosed subject and an ovarian cancer diagnosed subject in a reference population with at least 75% accuracy; or
d) IRF1, ICAM1, TIMP1, PTGS2, TGFB1, TNFRSF1A, CXCL1, or IFI16 wherein the constituent distinguishes between a breast cancer diagnosed subject and a cervical cancer diagnosed subject in a reference population with at least 75% accuracy;
e) ELA2, VEGF, TIMP1, PTPRC, MMP9, IL1R1, PTGS2, TXNRD1, IL10, HSPA1A, IL1RN, ALOX5, APAF1, CXCL1, TNF, MAPK14, or EGR1 wherein the constituent distinguishes between a breast cancer diagnosed subject and a lung cancer diagnosed subject in a reference population with at least 75% accuracy.

3. The method of claim 1, wherein said constituent is selected from Table B and is
a) EGR1, TGFB1, NFKB1, SRC, TP53, ABL1, SERPINE1, or CDKN1A wherein the constituent distinguishes between a breast cancer diagnosed subject and a melanoma cancer diagnosed subject in a reference population with at least 75% accuracy;
b) TIMP1, MMP9, CDKN1A, or IFITM1 wherein the constituent distinguishes between a breast cancer diagnosed subject and an ovarian cancer diagnosed subject in a reference population with at least 75% accuracy;
c) NME4, TIMP1, BRAF, ICAM1, PLAU, RHOA, IFITM1, TNFRSF1A, NOTCH2, TGFB1, SEMA4D, MMP9, FOS, TNF, MYC, AKT1, or EGR1 wherein the constituent distinguishes between a breast cancer diagnosed subject and a cervical cancer diagnosed subject in a reference population with at least 75% accuracy; or
d) BRAF, PLAU, RHOA, RB1, TIMP1, CDKN1A, SMAD4, S100A4, NME4, MMP9, IFITM1, PTEN, VEGF, NRAS, TNF, TGFB1, BRCA1, SEMA4D, CDK5, TNFRSF1A, or EGR1 wherein the constituent distinguishes between a breast cancer diagnosed subject and a lung cancer diagnosed subject in a reference population with at least 75% accuracy.

4. The method of claim 1, wherein said constituent is selected from Table C and is
a) TGFB1, EGR1, SMAD3, NFKB1, SRC, TP53, NFATC2, PDGFA, or SERPINE1, wherein the constituent distinguishes between a breast cancer diagnosed subject and a melanoma cancer diagnosed subject in a reference population with at least 75% accuracy;
b) ALOX5 or EP300 wherein the constituent distinguishes between a breast cancer diagnosed subject and an ovarian cancer diagnosed subject in a reference population with at least 75% accuracy;
c) ALOX5, CREBBP, EP300, MAPK1, ICAM1, PLAU, TGFB1, CEBPB, FOS, or SMAD3 wherein the constituent distinguishes between a breast cancer diagnosed subject and a cervical cancer diagnosed subject in a reference population with at least 75% accuracy; or
d) EP300, PLAU, MAPK1, ALOX5, CREBBP, TOPBP1, PTEN, S100A6, TGFB1, or EGR1, wherein the constituent distinguishes between a breast cancer diagnosed subject and a lung cancer diagnosed subject in a reference population with at least 75% accuracy.

5. The method of claim 1, wherein the said constituents are selected according to any of the models enumerated in
a) Table A1a, Table A2a, Table A3a, Table A8a or Table A18a;
b) Table B1a, Table B2a, Table B3a, Table B8a or Table B18a; or
c) Table C1a, Table C2a, Table C3a, or Table C8a.

6. A method for evaluating the presence of cervical cancer in a subject based on a sample from the subject, the sample providing a source of RNAs, comprising:
a) determining a quantitative measure of the amount of at least one constituent of any constituent of any one table selected from the group consisting of Tables A, B and C, as a distinct RNA constituent in the subject sample, wherein such measure is obtained under measurement conditions that are substantially repeatable and the constituent is selected so that measurement of the constituent distinguishes between a cervical cancer-diagnosed subject and a subject having a cancer selected from the group consisting of melanoma, lung, colon, ovarian and breast in a reference population with at least 75% accuracy.
b) comparing the quantitative measure of the constituent in the subject sample to a reference value.

7. The method of claim 6, wherein said constituent is selected from Table A and is
a) IFI16, LTA, TNFRSF1A, PTPRC, VEGF, TNF, TIMP1, CD86, PLAUR, PTGS2, ADAM17, MYC, TGFB1, IL1RN, HMOX1, TLR4, TLR2, MNDA, MAPK14, TXNRD1, ICAM1, CASP3, IL1B, CCL5, NFKB1, HLADRA, SSI3, SERPINA1, HSPA1A, MMP9, SERPINE1, MHC2TA, CXCR3, PLA2G7, CCR5, CD19, or EGR1 wherein the constituent distinguishes between a cervical cancer diagnosed subject and a colon cancer diagnosed subject in a reference population with at least 75% accuracy;
b) IFI16, PLAUR, TGFB1, TNFRSF1A, LTA, TIMP1, MAPK14, ICAM1, IL1RN, PTPRC, IL1B, ADAM17, PTGS2, CCL5, TNF, EGR1, SSI3, HMOX1, MYC, CD86, IRF1, MNDA, TLR2, NFKB1, SERPINE1, HSPA1A, SERPINA1, TXNRD1, MMP9, VEGF, TLR4, CASP3, CXCR3, CD4, CCL3, CASP1, MHC2TA, CCR5, TNFSF5, HLADRA, IL18BP, IL1R1, or IL32, wherein the constituent distinguishes between a cervical cancer diagnosed subject and a melanoma cancer diagnosed subject in a reference population with at least 75% accuracy;
c) LTA wherein the constituent distinguishes between a cervical cancer diagnosed subject and an ovarian cancer diagnosed subject in a reference population with at least 75% accuracy;
d) IRF1, ICAM1, TIMP1, PTGS2, TGFB1, TNFRSF1A, CXCL1, or IFI16 wherein the constituent distinguishes between a cervical cancer diagnosed subject and a breast cancer diagnosed subject in a reference population with at least 75% accuracy; or
e) CASP3, IL18, TXNRD1, or IFNG wherein the constituent distinguishes between a cervical cancer diagnosed subject and a lung cancer diagnosed subject in a reference population with at least 75% accuracy.

8. The method of claim 6, wherein said constituent is selected from Table B and is
a) NME4, BRAF, NFKB1, SMAD4, ABL2, RHOA, NOTCH2, TIMP1, TGFB1, SEMA4D, BCL2, CDK2, NRAS, RB1, CDK5, IL1B, or FOS wherein the constituent distinguishes between a cervical cancer diagnosed subject and a colon cancer diagnosed subject in a reference population with at least 75% accuracy;
b) EGR1, ICAM1, TGFB1, SERPINE1, NME4, NFKB1, SEMA4D, TIMP1, TNF, BRAF, NOTCH2, SRC, RHOA, IFITM1, FOS, CDKN1A, PLAUR, PLAU, TNFRSF1A, IL1B, E2F1, TP53, THBS1, MYC, ABL2, AKT1, MMP9, SOCS1, SMAD4, CDK5, CDK2, ABL1, RHOC, BRCA1, or BCL2 wherein the constituent distinguishes between a cervical cancer diagnosed subject and a melanoma cancer diagnosed subject in a reference population with at least 75% accuracy;
c) MYCL1 or AKT1 wherein the constituent distinguishes between a cervical cancer diagnosed subject and an ovarian cancer diagnosed subject in a reference population with at least 75% accuracy;
d) NME4, TIMP1, BRAF, ICAM1, PLAU, RHOA, IFITM1, TNFRSF1A, NOTCH2, TGFB1, SEMA4D, MMP9, FOS, TNF, MYC, AKT1, or EGR1 wherein the constituent distinguishes between a cervical cancer diagnosed subject and a breast cancer diagnosed subject in a reference population with at least 75% accuracy; or
e) ITGB1 or RB1 wherein the constituent distinguishes between a cervical cancer diagnosed subject and a lung cancer diagnosed subject in a reference population with at least 75% accuracy.

9. The method of claim 6, wherein said constituent is selected from Table C and is
a) EP300, ALOX5, MAPK1, CREBBP, NFKB1, ICAM1, SMAD3, TGFB1, CEBPB, TOPBP1, NR4A2, FOS, or EGR1 wherein the constituent distinguishes between a cervical cancer diagnosed subject and a colon cancer diagnosed subject in a reference population with at least 75% accuracy;
b) EGR1, ICAM1, PDGFA, TGFB1, EP300, SERPINE1, CREBBP, ALOX5, NFKB1, MAPK1, SRC, SMAD3, FOS, PLAU, CEBPB, TP53, THBS1, MAP2K1, NFATC2, NR4A2, EGR2, EGR3, TOPBP1, or CDKN2D wherein the constituent distinguishes between a cervical cancer diagnosed subject and a melanoma cancer diagnosed subject in a reference population with at least 75% accuracy;
c) ALOX5, CREBBP, EP300, MAPK1, ICAM1, PLAU, TGFB1, CEBPB, FOS, or SMAD3 wherein the constituent distinguishes between a cervical cancer diagnosed subject and a breast cancer diagnosed subject in a reference population with at least 75% accuracy; or
d) S100A6 wherein the constituent distinguishes between a cervical cancer diagnosed subject and a lung cancer diagnosed subject in a reference population with at least 75% accuracy.

10. The method of claim 6, wherein the said constituents are selected according to any of the models enumerated in
a) Table A3a, Table A4a, Table A5a, Table A6a or Table A9a;
b) Table B3a, Table B4a, Table B5a, Table B6a or Table B9a; or
c) Table C3a, Table C4a, Table C5a, Table C6a or Table C9a.

11. A method for evaluating the presence of lung cancer in a subject based on a sample from the subject, the sample providing a source of RNAs, comprising:
a) determining a quantitative measure of the amount of at least one constituent of any constituent of any one table selected from the group consisting of Tables A, B and C, as a distinct RNA constituent in the subject sample, wherein such measure is obtained under measurement conditions that are substantially repeatable and the constituent is selected so that measurement of the constituent distinguishes between a lung cancer diagnosed subject and a subject having a cancer selected from the group consisting of melanoma, breast, colon, ovarian, prostate and cervical in a reference population with at least 75% accuracy.
b) comparing the quantitative measure of the constituent in the subject sample to a reference value.

12. The method of claim 11, wherein said constituent is selected from Table A and is
a) LTA, CD86, IFI16, PTPRC, VEGF, ADAM17, TXNRD1, TNF, MNDA, TIMP1, HMOX1, PTGS2, TNFRSF1A, IL1RN, TLR4, MYC, IL10, MAPK14, TLR2, PLAUR, TGFB1, ELA2, PLA2G7, IL1R1, NFKB1, IL1B, IL18, CXCR3, IL15, CCL5, HLADRA, EGR1, HSPA1A, IL5, ICAM1, SSI3, or IL8 wherein the constituent distinguishes between a lung cancer diagnosed subject and a colon cancer diagnosed subject in a reference population with at least 75% accuracy;
b) IFI16, LTA, TIMP1, MAPK14, EGR1, ADAM17, PTPRC, HMOX1, CD86, TGFB1, CCL5, IL1RN, TNFRSF1A, TNF, PTGS2, IL1B, MNDA, PLAUR, TXNRD1, MYC, IL10, TLR2, SSI3, MMP9, VEGF, NFKB1, TLR4, ICAM1, SERPINE1, SERPINA1, HSPA1A, CXCR3, IL1R1, CCL3, IRF1, ELA2, CASP1, CCR5, CD4, IL18, MHC2TA, CXCL1, IL18BP, IL5, HLADRA, or TNFSF6 wherein the constituent distinguishes between a lung cancer diagnosed subject and a melanoma cancer diagnosed subject in a reference population with at least 75% accuracy;
c) CASP3 or APAF1 wherein the constituent distinguishes between a lung cancer diagnosed subject and an ovarian cancer diagnosed subject in a reference population with at least 75% accuracy;
d) CASP3, IL18, TXNRD1, or IFNG wherein the constituent distinguishes between a lung cancer diagnosed subject and a cervical cancer diagnosed subject in a reference population with at least 75% accuracy;
e) ELA2, VEGF, TIMP1, PTPRC, MMP9, IL1R1, PTGS2, TXNRD1, IL10, HSPA1A, IL1RN, ALOX5, APAF1, CXCL1, TNF, MAPK14, or EGR1 wherein the constituent distinguishes between a lung cancer diagnosed subject and a breast cancer diagnosed subject in a reference population with at least 75% accuracy; or
f) CCL5, EGR1, TGFB1, IL1RN, TIMP1, CCL3, TNF, PLAUR, IL1B, CXCR3, PTGS2, TNFRSF1A, PTPRC, NFKB1, ICAM1, CD8A, IRF1, IL32, HMOX1, SERPINA1, HSPA1A, or ALOX5 wherein the constituent distinguishes between a lung cancer diagnosed subject and a prostate cancer diagnosed subject in a reference population with at least 75% accuracy.

13. The method of claim 11, wherein said constituent is selected from Table B and is
a) BRAF, NME4, RB1, SMAD4, NFKB1, RHOA, BRCA1, APAF1, NRAS, PLAU, CDK5, VEGF, TIMP1, BCL2, RAF1, TGFB1, SEMA4D, CFLAR, NOTCH2, or ABL2 wherein the constituent distinguishes between a lung cancer diagnosed subject and a colon cancer diagnosed subject in a reference population with at least 75% accuracy;
b) EGR1, TGFB1, NFKB1, RHOA, BRAF, CDKN1A, TIMP1, TNF, PLAU, IFITM1, ICAM1, SEMA4D, THBS1, SERPINE1, NME4, NOTCH2, E2F1, SMAD4, MMP9, TP53, FOS, PLAUR, CDK5, IL1B, RB1, MYC, AKT1, SRC, TNFRSF1A, BRCA1, ABL2, PTCH1, CDK2, IGFBP3, CDC25A, SOCS1, WNT1, RHOC, PTEN, ITGB1, S100A4, ABL1, APAF1, VHL, or BCL2 wherein the constituent distinguishes between a lung cancer diagnosed subject and a melanoma cancer diagnosed subject in a reference population with at least 75% accuracy;
c) ITGB1 or RB1 wherein the constituent distinguishes between a lung cancer diagnosed subject and a cervical cancer diagnosed subject in a reference population with at least 75% accuracy;
d) BRAF, PLAU, RHOA, RB1, TIMP1, CDKN1A, SMAD4, S100A4, NME4, MMP9, IFITM1, PTEN, VEGF, NRAS, TNF, TGFB1, BRCA1, SEMA4D, CDK5, TNFRSF1A, or EGR1 wherein the constituent distinguishes between a lung cancer diagnosed subject and a breast cancer diagnosed subject in a reference population with at least 75% accuracy; or
e) EGR1, TGFB1, S100A4, RHOA, PLAUR, CDKN1A, TIMP1, WNT1, SEMA4D, E2F1, or SOCS1 wherein the constituent distinguishes between a lung cancer diagnosed subject and a prostate cancer diagnosed subject in a reference population with at least 75% accuracy.

14. The method of claim 11, wherein said constituent is selected from Table C and is
a) EP300, TOPBP1, ALOX5, NFKB1, MAPK1, CREBBP, PLAU, SMAD3, NAB1, MAP2K1, TGFB1, RAF1, or EGR1 wherein the constituent distinguishes between a lung cancer diagnosed subject and a colon cancer diagnosed subject in a reference population with at least 75% accuracy;
b) EGR1, TGFB1, EP300, PDGFA, NFKB1, CREBBP, ALOX5, MAPK1, PLAU, SMAD3, ICAM1, THBS1, SERPINE1, MAP2K1, TP53, TOPBP1, FOS, NFATC2, SRC, CEBPB, CDKN2D, NR4A2, PTEN, EGR2, or EGR3 wherein the constituent distinguishes between a lung cancer diagnosed subject and a melanoma cancer diagnosed subject in a reference population with at least 75% accuracy;
c) S100A6 wherein the constituent distinguishes between a lung cancer diagnosed subject and a cervical cancer diagnosed subject in a reference population with at least 75% accuracy;
d) EP300, PLAU, MAPK1, ALOX5, CREBBP, TOPBP1, PTEN, S100A6, TGFB1, or EGR1 wherein the constituent distinguishes between a lung cancer diagnosed subject and a breast cancer diagnosed subject in a reference population with at least 75% accuracy; or
e) EGR1, TGFB1, S100A6, EP300, or CREBBP wherein the constituent distinguishes between a lung cancer diagnosed subject and a prostate cancer diagnosed subject in a reference population with at least 75% accuracy.

15. The method of claim 11, wherein the said constituents are selected according to any of the models enumerated in
a) Table A8a, Table A9a, Table A10a, Table A11a, Table A12a or Table A13a;
b) Table B8a, Table B9a, Table B10a, Table B11a, Table B12a or Table B13a; or
c) Table C8a, Table C9a, Table C10a, Table C11a, Table C12a or Table C13a.

16. A method for evaluating the presence of ovarian cancer in a subject based on a sample from the subject, the sample providing a source of RNAs, comprising:
a) determining a quantitative measure of the amount of at least one constituent of any constituent of any one table selected from the group consisting of Tables A, B and C, as a distinct RNA constituent in the subject sample, wherein such measure is obtained under measurement conditions that are substantially repeatable and the constituent is selected so that measurement of the constituent distinguishes between an ovarian cancer diagnosed subject and a subject having a cancer selected from the group consisting of melanoma, lung, colon, breast and cervical in a reference population with at least 75% accuracy.
b) comparing the quantitative measure of the constituent in the subject sample to a reference value.

17. The method of claim 16, wherein said constituent is selected from Table A and is
a) LTA, IFI16, PTPRC, TNFRSF1A, TIMP1, MNDA, TLR2, IL1RN, VEGF, MAPK14, TLR4, TXNRD1, SSI3, PLAUR, PTGS2, TGFB1, HMOX1, IL1B, IL10, CASP3, ADAM17, or SERPINA1 wherein the constituent distinguishes between an ovarian cancer diagnosed subject and a colon cancer diagnosed subject in a reference population with at least 75% accuracy;
b) IFI16, MAPK14, TNFRSF1A, TIMP1, PTPRC, TGFB1, IL1B, SSI3, IL1RN, LTA, PLAUR, MNDA, HMOX1, TLR2, PTGS2, ICAM1, EGR1, TXNRD1, MMP9, TLR4, MYC, SERPINE1, SERPINA1, HSPA1A, VEGF, CCL5, NFKB1, IL10, ADAM17, TNF, IL1R1, CASP3, or CD86 wherein the constituent distinguishes between an ovarian cancer diagnosed subject and a melanoma cancer diagnosed subject in a reference population with at least 75% accuracy;
c) TIMP1, MAPK14, SSI3, PTPRC, or IL1RN wherein the constituent distinguishes between an ovarian cancer diagnosed subject and a breast cancer diagnosed subject in a reference population with at least 75% accuracy;
d) LTA wherein the constituent distinguishes between an ovarian cancer diagnosed subject and a cervical cancer diagnosed subject in a reference population with at least 75% accuracy; or
e) CASP3 or APAF1 wherein the constituent distinguishes between an ovarian cancer diagnosed subject and a lung cancer diagnosed subject in a reference population with at least 75% accuracy.

18. The method of claim 16, wherein said constituent is selected from Table B and is
a) TIMP1, IL1B, or RB1 wherein the constituent distinguishes between an ovarian cancer diagnosed subject and a colon cancer diagnosed subject in a reference population with at least 75% accuracy;
b) TGFB1, TIMP1, SERPINE1, NFKB1, RHOA, IL1B, IFITM1, EGR1, CDKN1A, ICAM1, SEMA4D, E2F1, MMP9, THBS1, BRAF, SRC, PLAU, TNFRSF1A, NOTCH2, NME4, FOS, PLAUR, MYC, or SOCS1 wherein the constituent distinguishes between an ovarian cancer diagnosed subject and a melanoma cancer diagnosed subject in a reference population with at least 75% accuracy;
c) TIMP1, MMP9, CDKN1A, or IFITM1 wherein the constituent distinguishes between an ovarian cancer diagnosed subject and a breast cancer diagnosed subject in a reference population with at least 75% accuracy; or
d) MYCL1 or AKT1 wherein the constituent distinguishes between an ovarian cancer diagnosed subject and a cervical cancer diagnosed subject in a reference population with at least 75% accuracy.

19. The method of claim 16, wherein said constituent is selected from Table C and is a) ALOX5 or EP300 wherein the constituent distinguishes between an ovarian cancer diagnosed subject and a colon cancer diagnosed subject in a reference population with at least 75% accuracy;
b) TGFB1, PDGFA, ALOX5, NFKB1, SERPINE1, EP300, ICAM1, CREBBP, EGR1, THBS1, SRC, PLAU, CEBPB, MAPK1, FOS, or CDKN2D wherein the constituent distinguishes between an ovarian cancer diagnosed subject and a melanoma cancer diagnosed subject in a reference population with at least 75% accuracy; or
c) ALOX5 or EP300 wherein the constituent distinguishes between an ovarian cancer diagnosed subject and a breast cancer diagnosed subject in a reference population with at least 75% accuracy.

20. The method of claim 16, wherein the said constituents are selected according to any of the models enumerated in
a) Table A2a, Table A6a, Table B12a, Table A14a or Table A15a;
b) Table B2a, Table B6a, Table B12a, Table B14a or Table B15a; or
c) Table C2a, Table C6a, Table C12a, Table C14a or Table C15a.

21. A method for evaluating the presence of prostate cancer in a subject based on a sample from the subject, the sample providing a source of RNAs, comprising:
a) determining a quantitative measure of the amount of at least one constituent of any constituent of any one table selected from the group consisting of Tables A, B and C, as a distinct RNA constituent in the subject sample, wherein such measure is obtained under measurement conditions that are substantially repeatable and the constituent is selected so that measurement of the constituent distinguishes between a prostate cancer diagnosed subject and a subject having a cancer selected from the group consisting of melanoma, lung, and colon in a reference population with at least 75% accuracy.
b) comparing the quantitative measure of the constituent in the subject sample to a reference value.

22. The method of claim 21, wherein said constituent is selected from Table A and is
a) IFI16, LTA, ADAM17, MAPK14, PTPRC, TLR4, TXNRD1, VEGF, TLR2, ELA2, GZMB, MNDA, TNFRSF1A, TIMP1, CD86, IL15, or HMOX1 wherein the constituent distinguishes between a prostate cancer diagnosed subject and a colon cancer diagnosed subject in a reference population with at least 75% accuracy;
b) IFI16, MAPK14, ADAM17, TIMP1, LTA, TLR2, TNFRSF1A, SSI3, PTPRC, TXNRD1, TGFB1, TLR4, EGR1, MYC, MNDA, IL1R1, IL1RN, HMOX1, MMP9, VEGF, IL1B, PTGS2, ELA2, SERPINE1, CD86, TNF, IL15, or MHC2TA wherein the constituent distinguishes between a prostate cancer diagnosed subject and a melanoma cancer diagnosed subject in a reference population with at least 75% accuracy; or
c) CCL5, EGR1, TGFB1, IL1RN, TIMP1, CCL3, TNF, PLAUR, IL1B, CXCR3, PTGS2, TNFRSF1A, PTPRC, NFKB1, ICAM1, CD8A, IRF1, IL32, HMOX1, SERPINA1, HSPA1A, or ALOX5 wherein the constituent distinguishes between a prostate cancer diagnosed subject and a lung cancer diagnosed subject in a reference population with at least 75% accuracy.

23. The method of claim 21, wherein said constituent is selected from Table B and is a) IL18, RB1 or ANGPT1 wherein the constituent distinguishes between a prostate cancer diagnosed subject and a colon cancer diagnosed subject in a reference population with at least 75% accuracy;
b) BRAF, EGR1, RB1, SERPINE1, NFKB1, or RHOA wherein the constituent distinguishes between a prostate cancer diagnosed subject and a melanoma cancer diagnosed subject in a reference population with at least 75% accuracy; or
c) EGR1, TGFB1, S100A4, RHOA, PLAUR, CDKN1A, TIMP1, WNT1, SEMA4D, E2F1, or SOCS1 wherein the constituent distinguishes between a prostate cancer diagnosed subject and a lung cancer diagnosed subject in a reference population with at least 75% accuracy.

24. The method of claim 21, wherein said constituent is selected from Table C and is
a) TOPBP1 wherein the constituent distinguishes between a prostate cancer diagnosed subject and a colon cancer diagnosed subject in a reference population with at least 75% accuracy;
b) EP300, EGR1, MAPK1, ALOX5, PLAU, SERPINE1, or NFKB1 wherein the constituent distinguishes between a prostate cancer diagnosed subject and a melanoma cancer diagnosed subject in a reference population with at least 75% accuracy; or
c) EGR1, TGFB1, S100A6, EP300, or CREBBP wherein the constituent distinguishes between a prostate cancer diagnosed subject and a lung cancer diagnosed subject in a reference population with at least 75% accuracy.

25. The method of claim 21, wherein the said constituents are selected according to any of the models enumerated in
a) Table A13a, Table A16a or Table A17a;
b) Table B13a, Table B16a or Table B17a; or
c) Table C13a, Table C16a or Table C17a.

26. A method for evaluating the presence of colon cancer in a subject based on a sample from the subject, the sample providing a source of RNAs, comprising:
a) determining a quantitative measure of the amount of at least one constituent of any constituent of any one table selected from the group consisting of Tables A, B and C, as a distinct RNA constituent in the subject sample, wherein such measure is obtained under measurement conditions that are substantially repeatable and the constituent is selected so that measurement of the constituent distinguishes between a colon cancer diagnosed subject and a subject having a cancer selected from the group consisting of melanoma, lung, ovarian, breast, prostate and cervical in a reference population with at least 75% accuracy.
b) comparing the quantitative measure of the constituent in the subject sample to a reference value.

27. The method of claim 26, wherein said constituent is selected from Table A and is
a) LTA, IFI16, PTPRC, CD86, ADAM17, HMOX1, TXNRD1, MYC, MHC2TA, MAPK14, TLR2, CD19, TNFRSF1A, TIMP1, TNF, IL23A, HLADRA, TLR4, PLAUR, PTGS2, PLA2G7, CCR5, or TOSO wherein the constituent distinguishes between a colon cancer diagnosed subject and a breast cancer diagnosed subject in a reference population with at least 75% accuracy;
b) TGFB1, CCL5, SSI3, TIMP1, EGR1, IFI16, or SERPINE1 wherein the constituent distinguishes between a colon cancer diagnosed subject and a melanoma cancer diagnosed subject in a reference population with at least 75% accuracy;
c) LTA, IFI16, PTPRC, TNFRSF1A, TIMP1, MNDA, TLR2, IL1RN, VEGF, MAPK14, TLR4, TXNRD1, SSI3, PLAUR, PTGS2, TGFB1, HMOX1, IL1B, IL10, CASP3, ADAM17, or SERPINA1 wherein the constituent distinguishes between a colon cancer diagnosed subject and an ovarian cancer diagnosed subject in a reference population with at least 75% accuracy;
d) IFI16, LTA, TNFRSF1A, PTPRC, VEGF, TNF, TIMP1, CD86, PLAUR, PTGS2, ADAM17, MYC, TGFB1, IL1RN, HMOX1, TLR4, TLR2, MNDA, MAPK14, TXNRD1, ICAM1, CASP3, IL1B, CCL5, NFKB1, HLADRA, SSI3, SERPINA1, HSPA1A, MMP9, SERPINE1, MHC2TA, CXCR3, PLA2G7, CCR5, CD19, or EGR1 wherein the constituent distinguishes between a colon cancer diagnosed subject and a cervical cancer diagnosed subject in a reference population with at least 75% accuracy; or
e) LTA, CD86, IFI16, PTPRC, VEGF, ADAM17, TXNRD1, TNF, MNDA, TIMP1, HMOX1, PTGS2, TNFRSF1A, IL1RN, TLR4, MYC, IL10, MAPK14, TLR2, PLAUR, TGFB1, ELA2, PLA2G7, IL1R1, NFKB1, IL1B, IL18, CXCR3, IL15, CCL5, HLADRA, EGR1, HSPA1A, IL5, ICAM1, SSI3, or IL8 wherein the constituent distinguishes between a colon cancer diagnosed subject and a lung cancer diagnosed subject in a reference population with at least 75% accuracy.
f) IFI16, LTA, ADAM17, MAPK14, PTPRC, TLR4, TXNRD1, VEGF, TLR2, ELA2, GZMB, MNDA, TNFRSF1A, TIMP1, CD86, IL15, or HMOX1 wherein the constituent distinguishes between a colon cancer diagnosed subject and a prostate cancer diagnosed subject in a reference population with at least 75% accuracy.

28. The method of claim 26, wherein said constituent is selected from Table B and is
a) EGR1, TGFB1, SERPINE1, E2F1, THBS1, IFITM1, or FGFR2, wherein the constituent distinguishes between a colon cancer diagnosed subject and a melanoma cancer diagnosed subject in a reference population with at least 75% accuracy;
b) TIMP1, IL1B, or RB1 wherein the constituent distinguishes between a colon cancer diagnosed subject and an ovarian cancer diagnosed subject in a reference population with at least 75% accuracy;
c) NME4, BRAF, NFKB1, SMAD4, ABL2, RHOA, NOTCH2, TIMP1, TGFB1, SEMA4D, BCL2, CDK2, NRAS, RB1, CDK5, IL1B, or FOS wherein the constituent distinguishes between a colon cancer diagnosed subject and a cervical cancer diagnosed subject in a reference population with at least 75% accuracy;
d) BRAF, NME4, RB1, SMAD4, NFKB1, RHOA, BRCA1, APAF1, NRAS, PLAU, CDK5, VEGF, TIMP1, BCL2, RAF1, TGFB1, SEMA4D, CFLAR, NOTCH2, or ABL2 wherein the constituent distinguishes between a colon cancer diagnosed subject and a lung cancer diagnosed subject in a reference population with at least 75% accuracy; or
e) IL18, RB1 or ANGPT1 wherein the constituent distinguishes between a colon cancer diagnosed subject and a prostate cancer diagnosed subject in a reference population with at least 75% accuracy.

29. The method of claim 26, wherein said constituent is selected from Table C and is
a) PDGFA, TGFB1, SERPINE1, EGR1, THBS1, SMAD3, or NFATC2 wherein the constituent distinguishes between a colon cancer diagnosed subject and a melanoma cancer diagnosed subject in a reference population with at least 75% accuracy;
b) ALOX5 or EP300 wherein the constituent distinguishes between a colon cancer diagnosed subject and an ovarian cancer diagnosed subject in a reference population with at least 75% accuracy;
c) EP300, ALOX5, MAPK1, CREBBP, NFKB1, ICAM1, SMAD3, TGFB1, CEBPB, TOPBP1, NR4A2, FOS, or EGR1 wherein the constituent distinguishes between a colon cancer diagnosed subject and a cervical cancer diagnosed subject in a reference population with at least 75% accuracy;
d) EP300, TOPBP1, ALOX5, NFKB1, MAPK1, CREBBP, PLAU, SMAD3, NAB1, MAP2K1, TGFB1, RAF1, or EGR1 wherein the constituent distinguishes between a colon cancer diagnosed subject and a lung cancer diagnosed subject in a reference population with at least 75% accuracy; or
e) TOPBP1 wherein the constituent distinguishes between a colon cancer diagnosed subject and a prostate cancer diagnosed subject in a reference population with at least 75% accuracy.

30. The method of claim 26, wherein the said constituents are selected according to any of the models enumerated in:
a) Table A4a, Table A1a, Table A10a, Table A14a, Table A16a or Table 18a;
b) Table B4a, Table B7a, Table B10a, Table B14a, Table B16a or Table B18a; or
c) Table C4a, Table C7a, Table C10a, Table C14a, or Table C16a.

31. A method for evaluating the presence of melanoma cancer in a subject based on a sample from the subject, the sample providing a source of RNAs, comprising:
a) determining a quantitative measure of the amount of at least one constituent of any constituent of any one table selected from the group consisting of Tables A, B and C, as a distinct RNA constituent in the subject sample, wherein such measure is obtained under measurement conditions that are substantially repeatable and the constituent is selected so that measurement of the constituent distinguishes between a colon cancer diagnosed subject and a subject having a cancer selected from the group consisting of lung, colon, ovarian, breast, prostate and cervical in a reference population with at least 75% accuracy.
b) comparing the quantitative measure of the constituent in the subject sample to a reference value.

32. The method of claim 31, wherein said constituent is selected from Table A and is
a) IFI16, TIMP1, MAPK14, LTA, TGFB1, HMOX1, TNFRSF1A, PTPRC, PLAUR, EGR1, ADAM17, TLR2, MYC, SSI3, TNF, CD86, IL1B, CCL5, MHC2TA, CXCR3, TXNRD1, PTGS2, ICAM1, IL1RN, SERPINE1, CD4, NFKB1, CCR5, TLR4, IL18BP, CCL3, HLADRA, MMP9, or IL32 wherein the constituent distinguishes between a melanoma cancer diagnosed subject and a breast cancer diagnosed subject in a reference population with at least 75% accuracy;
b) TGFB1, CCL5, SSI3, TIMP1, EGR1, IFI16, or SERPINE1 wherein the constituent distinguishes between a melanoma cancer diagnosed subject and a colon cancer diagnosed subject in a reference population with at least 75% accuracy;
c) IFI16, MAPK14, TNFRSF1A, TIMP1, PTPRC, TGFB1, IL1B, SSI3, IL1RN, LTA, PLAUR, MNDA, HMOX1, TLR2, PTGS2, ICAM1, EGR1, TXNRD1, MMP9, TLR4, MYC, SERPINE1, SERPINA1, HSPA1A, VEGF, CCL5, NFKB1, IL10, ADAM17, TNF, IL1R1, CASP3, or CD86 wherein the constituent distinguishes between a melanoma cancer diagnosed subject and an ovarian cancer diagnosed subject in a reference population with at least 75% accuracy;
d) IFI16, PLAUR, TGFB1, TNFRSF1A, LTA, TIMP1, MAPK14, ICAM1, IL1RN, PTPRC, IL1B, ADAM17, PTGS2, CCL5, TNF, EGR1, SSI3, HMOX1, MYC, CD86, IRF1, MNDA, TLR2, NFKB1, SERPINE1, HSPA1A, SERPINA1, TXNRD1, MMP9, VEGF, TLR4, CASP3, CXCR3, CD4, CCL3, CASP1, MHC2TA, CCR5, TNFSF5, HLADRA, IL18BP, IL1R1, or IL32 wherein the constituent distinguishes between a melanoma cancer diagnosed subject and a cervical cancer diagnosed subject in a reference population with at least 75% accuracy;
e) IFI16, LTA, TIMP1, MAPK14, EGR1, ADAM17, PTPRC, HMOX1, CD86, TGFB1, CCL5, IL1RN, TNFRSF1A, TNF, PTGS2, IL1B, MNDA, PLAUR, TXNRD1, MYC, IL10, TLR2, SSI3, MMP9, VEGF, NFKB1, TLR4, ICAM1, SERPINE1, SERPINA1, HSPA1A, CXCR3, IL1R1, CCL3, IRF1, ELA2, CASP1, CCR5, CD4, IL18, MHC2TA, CXCL1, IL18BP, IL5, HLADRA, or TNFSF6 wherein the constituent distinguishes between a melanoma cancer diagnosed subject and a lung cancer diagnosed subject in a reference population with at least 75% accuracy; or
f) IFI16, MAPK14, ADAM17, TIMP1, LTA, TLR2, TNFRSF1A, SSI3, PTPRC, TXNRD1, TGFB1, TLR4, EGR1, MYC, MNDA, IL1R1, IL1RN, HMOX1, MMP9, VEGF, IL1B, PTGS2, ELA2, SERPINE1, CD86, TNF, IL15, MHC2TA wherein the constituent distinguishes between a melanoma cancer diagnosed subject and a prostate cancer diagnosed subject in a reference population with at least 75% accuracy.

33. The method of claim 31, wherein said constituent is selected from Table B and is
a) EGR1, TGFB1, NFKB1, SRC, TP53, ABL1, SERPINE1, or CDKN1A wherein the constituent distinguishes between a melanoma cancer diagnosed subject and a breast cancer diagnosed subject in a reference population with at least 75% accuracy;
b) EGR1, TGFB1, SERPINE1, E2F1, THBS1, IFITM1, or FGFR2; wherein the constituent distinguishes between a melanoma cancer diagnosed subject and a colon cancer diagnosed subject in a reference population with at least 75% accuracy;
c) TGFB1, TIMP1, SERPINE1, NFKB1, RHOA, IL1B, IFITM1, EGR1, CDKN1A, ICAM1, SEMA4D, E2F1, MMP9, THBS1, BRAF, SRC, PLAU, TNFRSF1A, NOTCH2, NME4, FOS, PLAUR, MYC, or SOCS1 wherein the constituent distinguishes between a melanoma cancer diagnosed subject and an ovarian cancer diagnosed subject in a reference population with at least 75% accuracy;
d) EGR1, ICAM1, TGFB1, SERPINE1, NME4, NFKB1, SEMA4D, TIMP1, TNF, BRAF, NOTCH2, SRC, RHOA, IFITM1, FOS, CDKN1A, PLAUR, PLAU, TNFRSF1A, IL1B, E2F1, TP53, THBS1, MYC, ABL2, AKT1, MMP9, SOCS1, SMAD4, CDK5, CDK2, ABL1, RHOC, BRCA1, or BCL2 wherein the constituent distinguishes between a melanoma cancer diagnosed subject and a cervical cancer diagnosed subject in a reference population with at least 75% accuracy;
e) EGR1, TGFB1, NFKB1, RHOA, BRAF, CDKN1A, TIMP1, TNF, PLAU, IFITM1, ICAM1, SEMA4D, THBS1, SERPINE1, NME4, NOTCH2, E2F1, SMAD4, MMP9, TP53, FOS, PLAUR, CDK5, IL1B, RB1, MYC, AKT1, SRC, TNFRSF1A, BRCA1, ABL2, PTCH1, CDK2, IGFBP3, CDC25A, SOCS1, WNT1, RHOC, PTEN, ITGB1, S100A4, ABL1, APAF1, VHL, or BCL2 wherein the constituent distinguishes between a melanoma cancer diagnosed subject and a lung cancer diagnosed subject in a reference population with at least 75% accuracy; or
f) BRAF, EGR1, RB1, SERPINE1, NFKB1, or RHOA wherein the constituent distinguishes between a melanoma cancer diagnosed subject and a prostate cancer diagnosed subject in a reference population with at least 75% accuracy.

34. The method of claim 31, wherein said constituent is selected from Table C and is
a) TGFB1, EGR1, SMAD3, NFKB1, SRC, TP53, NFATC2, PDGFA, or SERPINE1 wherein the constituent distinguishes between a melanoma cancer diagnosed subject and a breast cancer diagnosed subject in a reference population with at least 75% accuracy;
b) PDGFA, TGFB1, SERPINE1, EGR1, THBS1, SMAD3, or NFATC2 wherein the constituent distinguishes between a melanoma cancer diagnosed subject and a colon cancer diagnosed subject in a reference population with at least 75% accuracy;
c) TGFB1, PDGFA, ALOX5, NFKB1, SERPINE1, EP300, ICAM1, CREBBP, EGR1, THBS1, SRC, PLAU, CEBPB, MAPK1, FOS, or CDKN2D wherein the constituent distinguishes between a melanoma cancer diagnosed subject and an ovarian cancer diagnosed subject in a reference population with at least 75% accuracy;
d) EGR1, ICAM1, PDGFA, TGFB1, EP300, SERPINE1, CREBBP, ALOX5, NFKB1, MAPK1, SRC, SMAD3, FOS, PLAU, CEBPB, TP53, THBS1, MAP2K1, NFATC2, NR4A2, EGR2, EGR3, TOPBP1, or CDKN2D wherein the constituent distinguishes between a melanoma cancer diagnosed subject and a cervical cancer diagnosed subject in a reference population with at least 75% accuracy;
e) EGR1, TGFB1, EP300, PDGFA, NFKB1, CREBBP, ALOX5, MAPK1, PLAU, SMAD3, ICAM1, THBS1, SERPINE1, MAP2K1, TP53, TOPBP1, FOS, NFATC2, SRC, CEBPB, CDKN2D, NR4A2, PTEN, EGR2, or EGR3 wherein the constituent distinguishes between a melanoma cancer diagnosed subject and a lung cancer diagnosed subject in a reference population with at least 75% accuracy; or
f) EP300, EGR1, MAPK1, ALOX5, PLAU, SERPINE1, or NFKB1 wherein the constituent distinguishes between a melanoma cancer diagnosed subject and a prostate cancer diagnosed subject in a reference population with at least 75% accuracy.

35. The method of claim 31, wherein the said constituents are selected according to any of the models enumerated in
a) Table A1a, Table A5a, Table A7a, Table A11a, Table A15a or Table A17a;
b) Table B1a, Table B5a, Table B7a, Table B11a, Table B15a or Table B17a; or
c) Table C1a, Table C5a, Table C7a, Table C11a, Table C15a or Table C17a.

36. The method of any one of claims 1, 6, 11, 16, 21, 26 or 31, wherein said reference value is an index value.

37. The method of any one of claims 1, 6, 11, 16, 21, 26 or 31, wherein the sample is selected from the group consisting of blood, a blood fraction, a body fluid, a cells and a tissue.

38. The method of any one of claims 1, 6, 11, 16, 21, 26 or 31, wherein the measurement conditions that are substantially repeatable are within a degree of repeatability of better than ten percent.

39. The method of any one of claims 1, 6, 11, 16, 21, 26 or 31, wherein the measurement conditions that are substantially repeatable are within a degree of repeatability of better than five percent.

40. The method of any one of claims 1, 6, 11, 16, 21, 26 or 31, wherein the measurement conditions that are substantially repeatable are within a degree of repeatability of better than three percent.

41. The method of any one of claims 1, 6, 11, 16, 21, 26 or 31, wherein efficiencies of amplification for all constituents are substantially similar.

42. The method of any one of claims 1, 6, 11, 16, 21, 26 or 31, wherein the efficiency of amplification for all constituents is within ten percent.

43. The method of any one of claims 1, 6, 11, 16, 21, 26 or 31, wherein the efficiency of amplification for all constituents is within five percent.

44. The method of any one of claims 1, 6, 11, 16, 21, 26 or 31, wherein the efficiency of amplification for all constituents is within three percent.

45. A kit for detecting breast cancer in a subject, comprising at least one reagent for the detection or quantification of any constituent measured according to claim 1 and instructions for using the kit.

46. A kit for detecting cervical cancer in a subject, comprising at least one reagent for the detection or quantification of any constituent measured according to claim 6 and instructions for using the kit.

47. A kit for detecting lung cancer in a subject, comprising at least one reagent for the detection or quantification of any constituent measured according to claim 11 and instructions for using the kit.

48. A kit for detecting ovarian cancer in a subject, comprising at least one reagent for the detection or quantification of any constituent measured according to claim 16 and instructions for using the kit.

49. A kit for detecting prostate cancer in a subject, comprising at least one reagent for the detection or quantification of any constituent measured according to claim 21 and instructions for using the kit.

50. A kit for detecting colon cancer in a subject, comprising at least one reagent for the detection or quantification of any constituent measured according to claim 26 and instructions for using the kit.

51. A kit for detecting melanoma cancer in a subject, comprising at least one reagent for the detection or quantification of any constituent measured according to claim 31 and instructions for using the kit.
